(19) United States
(12) Patent Application Publication
(10) Pub. No.: US 2023/0226218 A1
(43) Pub. Date: Jul. 20, 2023

(54) Red Cell Extracellular Vesicles (RCEVs) Containing Cargoes and Methods of Use and Production Thereof

(71) Applicant: ERYTECH PHARMA, LYON (FR)

(72) Inventors: Karine CHARTON, MIONNAY (FR); Agnès CIBIEL, LENTILLY (FR); Anaïs BELLEDANT, LYON (FR); Leidy Diana PIEDRAHITA ESPINOSA, SAINT-ETIENNE (FR); Baptiste Alain MARTINON, LYON (FR)

(21) Appl. No.: 17/924,284

(22) PCT Filed: May 11, 2021
(86) PCT No.: PCT/EP2021/062447

§ 371 Date: Nov. 9, 2022

Related U.S. Application Data
(60) Provisional application No. 63/023,218, filed on May 11, 2020.

(51) Int. Cl. A61K 48/00; A61K 9/127; A61K 35/18

(52) U.S. Cl. CPC A61K 48/0041; A61K 9/127; A61K 35/18

(57) ABSTRACT

The application relates to the use of loaded red blood cells (e.g. “RBCs”, “red cells” or “erythrocytes”) or red blood cell precursors to produce red cell extracellular vesicles (RCEVs) containing cargos, including cargos comprising biologically active ingredients. Notable red cell precursors include hematopoietic stem cells (HSCs), induced pluripotent stem cells (iPSCs), and reticulocytes. The cargo may comprise nucleic acids, proteins, small molecules, or components of a gene editing system, including CRISPR/Cas9. The RCEVs may be used to treat of diseases and disorders including autoimmune disorders, cancers, cardiovascular diseases, gastrointestinal diseases, genetic disorders, or inflammatory diseases. The RCEVs may also be used to carry antigens and or immune modulator, for use in eliciting immune or immune tolerance responses. Also provided are methods for producing cargo loaded RCEVs (CLRCEVs) by first loading cargo into red cells and then by vesiculating the cargo loaded red cells to yield the CLRCEVs.

### CROSS-REFERENCE TO RELATED APPLICATIONS AND INCORPORATION BY REFERENCE

This application claims the benefit of U.S. Provisional Patent Application No. U.S. 63/023,218, filed May 11, 2020, and incorporated by reference herein in its entirety. All other references cited herein are likewise incorporated by reference in the entireties.

### FIELD OF THE INVENTION

The present disclosure relates to the use of mature red blood cells (e.g. “RBCs”, “red cells” or “erythrocytes”), immature red cells (e.g. reticulocytes) or red cell precursors to produce red cell extracellular vesicles (RCEVs) containing biologically active ingredients. Notable red cell precursors include hematopoietic stem cells (HSCs), erythroblasts and induced pluripotent stem cells (iPSCs). The disclosure also relates to pharmaceutical compositions comprising RCEVs containing antigens, including viral and tumor antigens, and/or immune modulators (e.g. adjuvants, TLR agonists or STING agonists), therapeutic polypeptides (e.g. enzymes, antigen-binding fragments, and the like), and nucleic acids (e.g. viral vectors for gene delivery and/or replacement, RNA, miRNA, lncRNA, circular RNA, DNA, plasmids, and the like), or other components including CAS9, ASOS, contrast agent, small molecule, chemotherapeutic, antiviral, antibacterial, antithrombotic, antiparasitic, immunosuppressive, antidiabetic, etc.

### SUMMARY OF THE INVENTION

Several techniques are known to stimulate the production of red cell extracellular vesicles (RCEVs) from red cells (see e.g. US 2019/0054192 to City University of Hong Kong; US 2019/0224238 to Hubei Soundny). As regards using RCEVs to carry cargo, including active pharmaceutical ingredients (API), most research teams appear to be taking the following “post-loading” approach: 1) RCEVs are produced from red cells; 2) RCEVs are loaded with cargo; and 3) cargo-loaded RCEVs (CLRCEVs) are sufficiently isolated and/or purified and optionally cryopreserved/lyophilized. However, it was not previously known whether red cells loaded with cargo could be induced to produce therapeutically effective CLRCEVs that still contain a clinically effective amount of the cargo, including API. As used herein, “API” encompasses all types of biologically active ingredients, including but not solely, small molecules and biologics.

In particular, it was not known whether red cells subjected to hypotonic encapsulation (e.g. as disclosed in U.S. Pat. No. 8,617,840 to Erytech), could be subjected to subsequent and/or simultaneous vesiculation to produce CLRCEVs containing an effective amount of the originally-encapsulated cargo. Now that the invention has been disclosed, it is envisioned that any red cells containing a sufficient amount of cargo may be induced to form CLRCEVs as disclosed herein. Accordingly, in some embodiments, the red cells may be loaded with cargo using hypotonic loading processes, including but not solely those disclosed in U.S. Pat. No. 8,617,840 (to Erytech), US 2016/0051482 (to Erydel) and U.S. Pat. No. 10,213,492 (to St. Georges Hospital). In particular embodiments, the hypotonic loading process does not include subjecting the hypotonically treated red cells to restorative hypertonic resealing.

As discussed further below, it has been surprisingly determined that omission of the restorative hypertonic resealing step can significantly increase the number of CLRCEVs that can be produced per cargo-loaded red cell (CLRC). In view of this unexpected finding, it is envisioned that hypotonic conditions may be used to “prime” any CLRC (e.g. donated blood, cultured from hematopoietic stem cells, or cultured from induced-pluripotent stem cells) for subsequent vesiculation.

The invention is thus based in part upon the surprising finding that red cells previously loaded with a cargo, for example an API, can be induced to produce a large quantify of high quality CLRCEVs containing the cargo. And as discussed in more detail below, it was surprisingly found that CLRCEVs produced from previously encapsulated red cells were taken up more readily by macrophages as compared with unloaded RCEVs produced from mock-loaded red cells. Such data indicate an unforeseen benefit to loading red cells first and then subjecting the loaded red cells to vesiculation methods such as those disclosed herein. CLRCEVs made according to the disclosure are generally less than about 200, 190, 180, 170, or about 160 nm in diameter, which makes them an ideal drug delivery system for active ingredients that need to penetrate biological barriers, including the blood brain barrier and poorly vascularized solid tumors. The CLRCEVs of the present disclosure may be rapidly taken up by immune cells, particularly those present in the liver, spleen, and in many types of tumors. In some embodiments, the CLRCEVs may carry immune modulators that are able to activate and/or stimulate tumor-resident immune cells, particularly immune dormant cells, to mobilize and fight cancer cells.

In particular embodiments, the red cells may be loaded using any suitable encapsulation technology, including an hypotonic loading process. In more particular embodiments, the encapsulation technology is Erytech's proprietary ERYCAPS® encapsulation technology, which is generally described in U.S. Pat. No. 8,617,840 (to Erytech). When referencing various stages of the ERYCAPS® encapsulation process, “E100”=packed, leukoreduced red cells; “E300”=E100 mixed with Cargo (hypotonic conditions); “E350”=resealed E300 red cells before final wash step (CLRCs, hypertonic conditions); “E400”=E350 after final wash step. In some particular embodiments, the hypertonic conditions may be omitted, and the red cells in hypotonic conditions may be transferred directly to isotonic conditions (e.g. about 275 to about 300 mOsm). In some embodiments, hypotonic conditions may be used during vesiculation.

In some embodiments, “hypotonic” conditions may be characterized by an osmolality of about 270, 260, 250, 240, 230, 220, 210, 200, 190, 180, 170, 160, or 150 mOsmoles/kilogram water (mOsm) or lower and “hypertonic” conditions may be characterized by an osmolality of about 300 mOsm or higher, including about 310, 320, 330, 340, 350, 360, 370, 380, 390, 400, 410, 420, 430, 440, 450, 460, 470, 480, 490, 500, or higher. In some embodiments, “isotonic” may be characterized by a normal range for human plasma, which may be from about 275 to about 299 mOsm, or about 285 to about 295 mOsm. In some embodiments, red cells may be subjected only to the minimum degree of hypotonic conditions required to allow a given cargo to enter said red cells.

In other embodiments, the red cells may be loaded using any suitable loading/encapsulation technology, including those available now and those yet to be developed. Such approaches may include the use of endocytosis, electroporation, mechanical squeezing (e.g. US 2018/0201889 to SQZ Biotechnologies), other microfluidic and/or mechanical means (e.g. US 2018/0155669 to Indee; WO2019018497 to Harvard; US 2019/0071695 to Cellino; and WO 2019/123372 to Politecnico di Milano), cell penetrating peptide, genetic engineering (e.g. WO2021053243 to The University of Bristol), osmotic-based encapsulation and variations and/or combinations thereof. Osmotic approaches may include hypotonic dilution, hypotonic pre-swelling, hypotonic dialysis (e.g. Erytech's ERYCAPS® red cell encapsulation technology), and osmotic pulse/isotonic osmotic lysis, genetic approaches (e.g. US 2018/0135012 to Rubius), via cell-penetrating peptide (CPP), or lipid nanoparticle (e.g. WO 2019/040516 to Rubius). In any of these embodiments, the previously loaded red cells may be primed for subsequent vesiculation by subjecting them to hypotonic conditions.

In some embodiments, cargo may include API including but not limited to anti-inflammatory agents, antiviral agents, corticosteroids, nucleic acid or polypeptide-based aptamers, cyclic dinucleotide (CDN) STING agonists, cytokines, enzymes, glucocorticoids, hormones, immunomodulators, immunosuppressants, nucleic acids, nucleosides, nucleoside analogs, nucleotide analogs, oligonucleotides, oligopeptides, peptides, polypeptides, proteins, small molecules, or toxins. In some embodiments, the cargo may be an immunomodulator, for example one that induces in a host an immune response against tumors, particularly immune-suppressed and/or “cold” tumors.

In a first object, the disclosure provides compositions of cargo loaded red cell extracellular vesicles (CLRCEVs) and methods of use and production thereof.

In some embodiments, CLRCEVs may be produced by subjecting loaded red cells to certain mechanical stresses, including but not limited to those process(es) disclosed in WO 2019/002608 A1 (to the University of PARIS, CNRS, and GENETHON).

In other embodiments, CLRCEVs may be produced by subjecting loaded red cells to certain chemical and/or osmotic stresses, including but not limited to those process(es) disclosed in US 2019/0054192 A1 (to City University of Hong Kong).

In some embodiments, CLRCEVs may be produced by subjecting CLRCs to extrusion processes disclosed in U.S. Ser. No. 10/675,244B2 (to MDimune).

Loaded RCEVs (“CLRCEVs”) as disclosed herein may be isolated and/or purified by standard techniques, including but not limited to centrifugation (e.g. sucrose gradient and/or cushion, ultracentrifugation, etc.), filtration (e.g. tangential flow filtration), chromatography (e.g. size exclusion, affinity, and the like), and combinations thereof. Isolated and/or purified RCEVs may then be maintained as a formulation suspended in a preservative liquid, or they may be lyophilized (or otherwise preserved) until reconstituted at around the time of administration to a subject or patient in need of administration therewith. In some embodiment, isolation and/or purification may be carried out using a high throughput system such as that described by McNamara et al. (J Extracell Vesicles. 2018_7 (1)). In such embodiments, CLRCEVs may be isolated, for example, by using cross-flow based filtration combined with high-molecular weight Capto Core (Sigma-Aldrich) size exclusion. CLRCEVs isolated using this or other comparable size exclusion methods may be subjected to further processing, including concentration and/or immune-purification.

In some embodiments, the loaded RCEVs may have a substantially similar membrane topology as compared with the originally loaded red cells (hereinafter “inside-in RCEVs” or “inside-in CLRCEVs”). In other embodiments, the loaded RCEVs may have a substantially reversed membrane topology as compared with the originally loaded red cells (hereinafter “inside-out RCEVs” or “inside-out CLRCEVs”). The inside-in or inside-out RCEVs/CLRCEVs may be further may be purified to produce pluralities of RCEVs wherein at least 90% of the RCEVs are either inside-out or inside-in relative to the originally loaded red cells. It is envisioned that when administered to a subject or patient in need thereof, the inside-out RCEVs may be cleared more rapidly by the reticuloendothelial system (RES) versus the inside-in RCEVs. The relative membrane topology may be measured using a variety of techniques, for example, by evaluating known markers of red cell internal and external surfaces.

In some embodiments, the CLRCEVs may be enriched for markers that are not routinely associated with red blood cells. For example, such markers may be those that are normally present in undetectable in red cells or those that are added during the process of encapsulation and/or vesiculation. In some embodiments, rapid uptake by immune cells (e.g. components of the reticuloendothelial system, “RES”) may be desired, and suitable “eat me” signals (e.g. phosphatidyl serine, “PS”) may be incorporated into the CLRCEVs of the disclosure. In alternative embodiments, less rapid uptake by cells may be desired, and “don't eat me” signals (e.g. CD47) may be incorporated into the CLRCEVs of the disclosure.

In some embodiments, increasing the proportion of inside-in CLRCs may yield a higher percentage of inside-in CLRCEVs. In such embodiments, Applicant's ERYCAPS® encapsulation system may be usefully applied, since it has been shown that ERYCAPS®-loaded CLRCs are substantially similar to the red cells prior to having been loaded with cargo (e.g. see “Comprehensive proteomic profiling of erythrocytes following hypotonic dialysis-based drug encapsulation process”, 22nd meeting of the Euro. Red Cell Research Society, 2019). Moreover, CLRCs produced using the ERYCAPS® system have similar half-lives and metabolic capabilities (among other characteristics), relative to their native red cell counterparts. Accordingly, when metabolically healthy encapsulated red cells are desired, as is the case where the CLRCs will be used as “bioreactors” (e.g. Erytech's ERYASPASE™ product, which is asparaginase encapsulated in red blood cells), gentle ERYCAPS® encapsulation is desirable. In some embodiments, gentle encapsulation comprises the steps of hypotonic swelling, loading of active ingredient, and restorative hypertonic resealing. In some gentle encapsulation embodiments, the hypertonic resealing step may be performed using a solution designed to restore the red cells to a metabolically healthy state after having subjected them to the stressful hypotonic conditions. As such, gentle encapsulation methods typically include a restorative, hypertonic resealing step.

In some alternative embodiments, for example, those in which a rapid and extensive vesiculation of the CLRCs is desired, less gentle encapsulation methods may be employed. For example, the hypotonic process may be modified to be more aggressive to “tune” or “prime” the CLRCs for vesiculation. For example, as discussed further below, stable, metabolically healthy encapsulated red cells (e.g. mock-loaded “proRBC” that have been subjected to the restorative, hypertonic resealing step) do not yield as many EVs per source red cell as compared to red cells that were identically encapsulated, but then transferred directly into isotonic conditions (i.e. about 275 to about 300 mOsm) after hypotonic encapsulation. As used herein, red cells that have been loaded (or mock-loaded) using hypotonic conditions but not later subjected to hypertonic resealing conditions are referred to as “cargo-wproRBC” (for CLRCs) or “wproRBC” (for mock-loaded red cells). And now that the disclosure has been made, the skilled artisan can routinely adjust the severity of the hypotonic encapsulation process to prime the loaded (or mock-loaded) red cells for rapid and extensive vesiculation. It is envisioned that more severe loading conditions may also permit the loading of larger and/or larger numbers of active ingredients, since the subsequent vesiculation step does not depend on the CLRCs being substantially similar to healthy, circulating red blood cells.

In other embodiments, the inside-in or inside-out RCEVs may be immune- or otherwise affinity-purified to yield populations of RCEVs of either desired topology. For example, inside-out RCEVs may be reversibly and selectively bound to elements of a purification system that bind to phosphatidylserine (PS).

In some embodiments, the beginning red cells comprise on average at least about 5,000, 50,000, 100,000, 150,000, 200,000, 1,000,000 or more molecules of cargo or active ingredient per red cell.

In some embodiments, the resulting CLRCEVs comprise on average at least about 500, 5,000, 10,000, 15,000, 20,000, 100,000 or more molecules of active ingredient per RCEV.

In some embodiments, RCEVs containing no cargo are produced by beginning with non-cargo loaded red cells. Such red cells may be sourced from donors (e.g. blood banks) or grown in culture beginning with red cell precursor cells (e.g. HSCs, erythroblasts, etc.). In some embodiments, unloaded RCEVs may be loaded with a cargo post-production using known (or yet to be developed) techniques for loading vesicles, including RCEVs. Some loading techniques include electroporation, sonoporation, other acoustic-based loading, hypotonic loading, transfection, laser-assisted transfection, mechanical loading including microfluidic-based loading (e.g. cell squeezing or vortex shedding), and cell penetrating peptide (CPP). In some embodiments, RCEVs may be produced and then loaded using the ERYCAPS® platform or an appropriately modified version thereof.

In some embodiments, the red cells or red cell precursors may be modified or engineered to express the cargo or to express and/or present targeting moieties. Engineered red cells may also comprise chimeric antigen receptor (CAR) or other multifunctional surface molecules.

In a second object, the disclosure provides methods of treating diseases including cancers, liver diseases (hemochromatosis, chronic hepatitis, hepatic tumor), thrombosis, excess iron, parasitic diseases, viral and/or bacterial infection, diabetes, cardiovascular disease, osteoporosis, anemia, lung infection, respiratory diseases, coagulopathies, and immune disorders, comprising administration of an effective amount of the RCEVs or CLRCEVs disclosed herein. In some embodiments, the RCEVs or CLRCEVs may be used to treat cancers of the liver, spleen, bone marrow, lung, lymph node, and/or to deliver active ingredients across biological barriers (e.g. the BBB or hypoxic and poorly vascularized regions of solid tumors).

In a third object, the disclosure provides kits comprising effective amounts of preserved and loaded RCEVs, optionally including instructions for use thereof in treating cancers.

In some embodiments, the RCEVs or CLRCEVs may be useful in treating one or more of the following: cancer selected from ALL, AML, adrenocortical adenoma, anaplastic thyroid cancer, bladder cancer, bone cancer, brain cancer, breast cancer, CLL, chondrosarcoma, colon cancer, colorectal cancer (CRC), DLBCL, Ewing sarcoma, fallopian tube cancer, gallbladder cancer, gastrointestinal/stomach (GIST) cancer, glioblastoma, glioma, hepatoblastoma, hepatocellular carcinoma (HCC), hepatocholangiocarcinoma, kidney cancer, leukemia, liver cancer, lung cancer, lymphoma, medulloblastoma, myeloma, nasopharyngeal cancer, neurofibromatosis-1 associated malignant peripheral nerve sheath tumors (MPNST), osteosarcoma, ovarian cancer, ovarian epithelial cancer, pancreatic cancer, pancreatic ductal carcinoma or pancreatic adenocarcinoma, papillary serous cystadenocarcinoma, prostate cancer, rectal cancer, renal cell carcinoma, rhabdomyosarcoma, salivary gland cancer, soft tissue and bone synovial sarcoma, squamous cell carcinoma of the head and neck (SCCHN), testicular cancer, uterine papillary serous carcinoma (UPSC) or Waldenstrom's macroglobulinemia. And since CLRCEVs may distribute preferentially to the spleen and to the liver, it is envisioned diseases and/or conditions affecting these organs may be effectively treated using CLRCEVs containing suitable active ingredients. For example, an effective amount of CLRCEVs containing an effective amount of an active ingredient (e.g. an immune modulator, a chemotherapeutic agent, and/or a small molecule) may be used to treat a splenic or hepatic cancer. In embodiments, the CLRCEVs are particularly effective against diseases of the spleen or liver.

In a fourth object, the disclosure provides methods of manufacture of a medicament comprising an effective amount of active ingredient loaded RCEVs.

In some embodiments, the RCEVs are loaded with and/or contain intravesicularly any one or more of the following active ingredients: nucleic acids, nucleotides, proteins, enzymes, small molecules, antibodies, siRNAs, miRNAs, cyclic dinucleotides (CDNs), adjuvants, immune modulators, kinase inhibitors, etc. In other embodiments, the RCEVs may be loaded with mRNAs encoding protein antigens for use in the preparation of vaccine compositions. RCEVs may also be loaded with mRNAs encoding proteins suitable for use in enzyme replacement therapy (ERT) or gene therapy applications.

It is a further object of the invention to not encompass within the invention any previously known product, process of making the product, or method of using the product such that the Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product.

These and other embodiments are disclosed or are obvious from and encompassed by the following Detailed Description.

### BRIEF DESCRIPTION OF THE DRAWINGS

FIG. 1 is a representative model of the platform (“EryVIP”) used to produce cargo loaded red cell extracellular vesicles (CLRCEVs) according to the disclosure;

FIG. 2 is a graph showing that cargo is present in the RCEVs produced from hypotonically loaded red cells. Lanes: (1) protein standards, (2) packed red cells from blood bank (day 3 after rinsing), (3) batch 1 CLRCs (day 1 after loading), (4) batch 2 CLRCs (day 1 after loading), (5) batch 1 CLRCs (D3), (6) batch 2 CLRCs (D3), (7) batch 1 CLRCs (D7), (8) batch 1 CLRCs (D14), (9) batch 1 CLRCs (D23), (10) empty, (11) positive control for presence of cargo;

FIG. 3 is a graph showing a trend whereby the number of RCEVs produced correlates with the proportion of phosphatidyl serine (PS) content. (1) “proRBC”=red cells processed using the ERYCAPS® device methods and (2) the number of microparticles/EVs=the amount produced prior to subsequent additional vesiculation (e.g. using the mechanical and/or chemical vesiculation methods as disclosed herein);

FIG. 4 is a graph showing mean tumor volume at the indicated days post tumor induction. Groups: mock red cell (G1), non-encapsulated (naked) 2 mg/kg STING agonist (G2), red cell-encapsulated 0.05 mg/kg CDN STING agonist (G3), and red cell-encapsulated 0.2 mg/kg CDN (G4). Based upon the efficacy of the red cell-encapsulated CDN, it is estimated that red cell encapsulation significantly reduces the amount of CDN required to elicit an immune response comparable to nearly 10-fold more naked CDN. Darker downward arrows=Ery-CDN administrations; lighter arrows=naked CDN administrations;

FIG. 5 is a graph showing median OVA tumor volume 15 days post OVA tumor seeding (treatments administered 3-& 7-days post seeding). Treatment groups: red cells encapsulating a low amount of P(IC) adjuvant plus OVA (OVA-PIC-high-RC, G1); OVA-RC plus separate administration of a P(IC)-high (G2); OVA-RC plus P(IC)-low (G3); OVA-RC (G4); unloaded RC (vehicle, G5);

FIG. 6 is a graph showing the yield in number of CLRCEV/CLRC when vesiculation was performed at low (LS), medium (MS), and high (HS) agitation speeds for 0, 2, 4, or 6 hours, or after ultracentrifugation (UC). LS=10%×MS and HS=MS+40%;

FIG. 7 are NTA plots presenting the concentrations and sizes of EVs vesiculated for 6 h;

FIG. 8 is a plot presenting the size distribution of FITC-dextran CLRCEVs;

FIG. 9 is a graph showing the portion of EVs loaded with detectable levels of FITC;

FIG. 10 is a graph showing the total number of STINGa-CLRCEVs (ERY-STINGa-2-S1 and ERY-STINGa-2-S1 batches) vs. the number of mock-loaded CLRCEVs (ERY-proRBC-1-S1, ERY-proRBC-1-S2 and ERY-proRBC-1-S3 batches) produced by applying about 85% medium speed agitation to either STINGa-loaded or mock-loaded red cells for 0, 2, 4, or 6 hours. Red cells used for the production of STINGa-loaded and mock-loaded red cells came from the same human donor;

FIG. 11 is a graph showing the number of mock-loaded CLRCEVs (made from wproRBC, ERY-wproRBC-1-S1 batch) vs. the number of mock-loaded CLRCEVs (made from proRBC, ERY-proRBC-1-S1 to S3 batches) produced by applying about 85% medium speed agitation to either mock-loaded wproRBC or mock-loaded proRBC for 0, 2, 4, or 6 hours, and also after purification by Amicon filtration. For wproRBC, red cells were subjected to hypotonic encapsulation conditions but not hypertonic resealing conditions during the mock encapsulation process. Notes: “wproRBC” means red cells that have been subjected to hypotonic encapsulation followed by direct return to physiological, isotonic conditions, without first being subjected hypertonic resealing conditions (which is the case for proRBC). Red cells used for the production of wproRBC and proRBC came from the same human donor;

FIG. 12 is a schematic diagram showing the scientific rationale behind the pHrodo® in vitro phagocytosis assay. Process: (1) pHrodo® labeled cells added to phagocytic cells (little to no fluorescence at ^{˜}pH 7.4 extracellular environment); (2) phagocytosis Initiated following receptor activation (formation of the phagocytic cup); and (3) formation of the phagosome (the acidic environment of the phagosome leads to increased red fluorescence);

FIG. 13 is graph showing the fluorescence produced by THP1-derived macrophages seeded at 24K, 34K or 44k cells/well, differentiated using 10, 20 and 100 ng/ml of PMA, and treated with EVs−/+pHrodo labeling. The red object intensity emitted per well was measured over twelve (12) hours;

FIG. 14 is a graph showing the mean red fluorescence present in THP1-derived macrophages differentiated using 100 ng/ml of PMA and treated with EVs-STINGa-pHrodo, control+pHrodo without EVs, and control EVs without pHrodo;

FIG. 15 are micrographs showing the uptake by THP1-derived macrophages differentiated using 100 ng/mL PMA and treated with EVs-STINGa+pHrodo (left) and pHrodo without EVs (right);

FIG. 16 is a graph showing the red fluorescence detected in THP1-derived macrophages after addition of EVs-STINGa 0.28 μg/ml 50k EVs/cell; EVS-proRBC 50k EVs/cell; and STINGa 0.28 μg/mL labeled with pHrodo. Control conditions included THP1+pHrodo dye and THP1 without pHrodo dye. Data points reflect the mean of n=3 wells and error bars show the standard deviation for each time point;

FIG. 17 are micrographs showing the difference of red fluorescence detected after 48 h in THP1-derived macrophages treated with A) control pHrodo; B) free STINGa 0.28 μg/ml+pHrodo; C) EVs-STINGa 0.28 μg/ml 50k EVs/cell labeled with pHrodo; D) EVS-proRBC 50k EVs/cell labeled with pHrodo;

FIG. 18 is a graph showing the red fluorescence detected in THP1-derived macrophages after addition of EVs-STINGa 0.14 μg/ml 25k EVs/cell; EVS-proRBC 25k EVs/cell labeled with pHrodo; and STINGa 0.14 μg/mL labeled with pHrodo. Control conditions included cells treated only with pHrodo or not. Data points reflect the mean of n=3 wells and error bars show the standard deviation for each time point;

FIG. 19 are micrographs showing the difference of red fluorescence detected after 48 h in THP1-derived macrophages after application of the following: A) pHrodo control; B) Free STINGa 0.14 μg/ml+pHrodo; C) EVs-STINGa 0.14 μg/ml 25k EVs/cell labeled with pHrodo; and D) EVs-proRBC 25k EVs/cell labeled with pHrodo;

FIG. 20 is a graph showing the red fluorescence detected in THP1-derived macrophages after addition of EVs-STINGa 0.03 μg/ml 5k EVs/cell; EVs-proRBC 5k EVs/cell and STINGa 0.03 μg/mL labeled with pHrodo; control conditions including cells only with pHrodo dye and THP1 without pHrodo were included. Data points reflect the mean of n=3 wells and error bars show the standard deviation (SD);

FIG. 21 are micrographs showing the difference of red fluorescence detected after 48 h in THP1-derived macrophages in: A) control condition with pHrodo; B) free STINGa 0.03 μg/ml; C) EVs-STINGa 0.03 μg/ml 5k EVs/cell labeled with pHrodo; and D) EVs-proRBC 5k EVs/cell labeled with pHrodo;

FIG. 22 is a graph showing the red fluorescence detected in undifferentiated THP1 monocytes after addition of EVs-STINGa 0.06 μg/ml 5k EVs/cell labeled with pHrodo; EVS-proRBC 5k EVs/cell; and STINGa 0.06 μg/mL+pHrodo. Control conditions included cells exposed only to pHrodo and cells without pHrodo. Data points reflect the mean of n=3 wells and error bars show SD;

FIG. 23 are micrographs showing the difference of red fluorescence detected after 48 h in THP1 undifferentiated monocytes after addition of A) control with pHrodo; B) free STINGa 0.06 μg/ml+pHrodo; C) EVs-STINGa 0.06 μg/ml 5k EVs/cell+pHrodo; and D) EVS-proRBC 5k EVs/cell+pHrodo;

FIG. 24 is a graph showing the uptake capacity differentiated THP1 macrophages vs. undifferentiated THP1 monocytes seeded at 50k/well and treated with 5k EVs/cell. Bars represent mean fluorescence detected at 48 h after application of the following (left to right): EVs-STINGa 0.03/mL; EVs-proRBC+pHrodo; STINGa 0.03/mL+pHrodo; control pHrodo; control without pHrodo; EVs-STINGa 0.06/mL+pHrodo; EVs-proRBC+pHrodo; control pHrodo; STINGa 0.06/mL; control without pHrodo;

FIG. 25 is a graph showing the amount of luciferase activity produced by THP1-Dual™ monocytes treated with free STING agonist (“STINGa”) (0.5, 1.0, 1.5, and 2.0 μg/mL), EVs-STINGa (also referred to as “ERY-STINGa-CLRCEV” or “STINGa-CLRCEV” throughout) (0.5, 1.0, 1.5, and 2.0 μg/mL, carried by the indicated amounts of EVs), and EVs-proRBC at the 24-hour time point. Briefly, 100k cells/well were seeded and the cells were treated with the indicated substances. After 24 h, the supernatants were recovered and subjected to QUANTI-Luc™ detection according to the manufacturer's instructions;

FIG. 26 is a graph showing the amount of IFN-β produced by THP1 macrophages at 6 h and 24 h after the application of STINGa (8 μg/mL); STINGa (3 μg/mL); EVs-STINGa (8 μg/mL, 2.0E11 EVs/mL); EVs-STINGa (3 μg/mL, 7.6E10 EVs/mL); EVs-proRBC (2.0E11 EVs/mL); EVs-proRBC (7.6E10 EVs/mL);

FIG. 27 is a graph showing the amount of IL-6 produced in THP1 macrophages at 6 h and 24 h after the application of STINGa (8 μg/mL); STINGa (2 μg/mL); STINGa (1 μg/mL); EVs-STINGa (8 μg/mL, 2.0E11 EVs/mL); EVs-STINGa (2 ug/mL, 5.0E10 EVs/mL); EVs-STINGa (1 ug/mL, 2.5E10 EVs/mL); EVs-proRBC (2.0E11 EVs/mL); EVs-proRBC (5.0E10 EVs/mL); EVs-proRBC (2.5E10 EVs/mL); or LPS (100 ng/mL);

FIG. 28 is a graph showing the amount of IL-1b produced in THP1 macrophages at 6 h and 24 h after the application of STINGa (8, 2, or 1 μg/mL); EVs-STINGa (8 μg/mL, 2.0E11 EVs/mL; 2 ug/mL, 5.0E10 EVs/mL; or 1 ug/mL, 2.5E10 EVs/mL); EVs-proRBC (2.0E11, 5.0E10, or 2.5E10 EVs/mL; or LPS (100 ng/mL);

FIG. 29 is a graph showing the amount of TNF-α produced in THP1 macrophages at 6 h and 24 h after the application of STINGa (8, 2, or 1 μg/mL); EVs-STINGa (8 μg/mL, 2.0E11 EVs/mL; 2 ug/mL, 5.0E10 EVs/mL; or 1 ug/mL, 2.5E10 EVs/mL); EVs-proRBC (2.0E11, 5.0E10, or 2.5E10 EVs/mL); or LPS;

FIG. 30 is a graph showing the number of EVs per RBC produced by the following conditions: (1) T0H-AS3; (2) T18H-AS3; (3) T28H-AS3; (4) T0H-AS3-Sonication; (5) T18H-AS3-Sonication; (6) T28H-AS3-Sonication; (7) T0H-PBS-Sonication; (8) T18H-PBS-Sonication; (9) T28H-PBS-Sonication; (10) TO-PBS; (11) T18-PBS; (12) T28-PBS; (13) T0H-Ca ionophore; (14) T18H-Ca ionophore; (15) T28H-Ca ionophore; (16) T0H-Ca ionophore-sonication; (17) T18H-Ca ionophore-sonication; (18) T28H-Ca ionophore-sonication. For wERY-FITC, red cells were subjected to hypotonic encapsulation conditions but not hypertonic resealing conditions during the encapsulation process. Red cells used for the production of wERY-FITC and ERY-FITC came from the same human donor;

FIG. 31 is a graph showing the mean particle size of EVs produced using the following conditions: (1) T0H-AS3; (2) T18H-AS3; (3) T28H-AS3; (4) T0H-AS3-Sonication; (5) T18H-AS3-Sonication; (6) T28H-AS3-Sonication; (7) T0H-PBS-Sonication; (8) T18H-PBS-Sonication; (9) T28H-PBS-Sonication; (10) TO-PBS; (11) T18-PBS; (12) T28-PBS; (13) T0H-Ca ionophore; (14) T18H-Ca ionophore; (15) T28H-Ca ionophore; (16) T0H-Ca ionophore-sonication; (17) T18H-Ca ionophore-sonication; (18) T28H-Ca ionophore-sonication. For wERY-FITC, red cells were subjected to hypotonic encapsulation conditions but not hypertonic resealing conditions during the encapsulation process. Red cells used for the production of wERY-FITC and ERY-FITC came from the same human donor;

FIG. 32 are Western blots probed with ALIX- or TSG101-specific antibodies. Lanes: (1) ladder; (2) unloaded red cells; (3) mock-loaded proRBC; (4) ERY-STINGa (red cells encapsulated with STINGa using ERYCAPS®; (5) EV-ERY-STINGa, S1 ERY04 batch; (6) EV-ERY-STINGa, S1 ERY06 batch; (7) EV-proRBC 51 ERY06 batch; (8) EV-proRBC S1 ERY07 batch; (9) EV-wproRBC S1 ERY04 batch; (10) ladder;

FIG. 33 are Western blots probed with HSP90- or β-actin-specific antibodies. Lanes: (1) ladder; (2) unloaded red cells; (3) mock-loaded proRBC; (4) ERY-STINGa (red cells encapsulated with STINGa using ERYCAPS®; (5) EV-ERY-STINGa, S1 ERY04 batch; (6) EV-ERY-STINGa, S1 ERY06 batch; (7) EV-proRBC S1 ERY06 batch; (8) EV-proRBC S1 ERY07 batch; (9) EV-wproRBC S1 ERY04 batch; (10) ladder.

### DETAILED DESCRIPTION OF THE INVENTION

Aspects of the present invention will now be discussed with reference to the accompanying figures. Further aspects will be apparent to those skilled in the art. All documents cited in this text are incorporated herein by reference in their entireties.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

Unless otherwise explained, all technical and scientific terms used herein have the same meaning as commonly understood by one of ordinary skill in the art to which this disclosure belongs. The singular terms “a”, “an”, and “the” include plural referents unless context clearly indicates otherwise. Similarly, the word “or” is intended to include “and” unless the context clearly indicates otherwise.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The term “effective amount” refers to an amount that provides a therapeutic effect for a given disease and/or condition in the context of a given administration regimen. This is a predetermined quantity of active material and/or drug substance (e.g. CLRCEV containing API) calculated to produce a desired biologic and/or therapeutic effect in conjunction with any required carrier(s), vehicle(s), additive(s), and/or diluent(s). Moreover, “effective amount” is intended to encompass an amount that is sufficient to diminish and/or prevent a clinically significant deficit in the activity, function and/or response of a patient or subject in need of such administration regimen. In the alternative, a therapeutically effective amount may be an amount sufficient to cause an improvement in a clinically significant condition in a subject or patient. And as a skilled artisan understands and appreciates, the amount of an active material containing API may vary depending on a variety of factors, notably including the specific activity and/or potency of the API. As such, appropriate dosages may comprise a predetermined amount of active material and/or API calculated to produce a desired therapeutic effect, optionally further comprising any required carrier, vehicle, and/or diluent. It is further intended that a therapeutically effective amount of active material may be provided in the practice of the uses for manufacture and methods of treatment disclosed herein. A therapeutically effective amount may easily be determined by a skilled medical or veterinary practitioner, based on subject or patient characteristics, including age, sex, weight, physical condition, comorbidities, and the like. Likewise, the skilled practitioner may administer a therapeutically effective dose by single and/or multiple administrations of subdivided doses at specific intervals. Alternatively, an effective dose may be administered as a continuous drip or infusion over an extended duration of time. Such extended administration may also be accomplished by the use of implantable devices comprising the active materials described herein.

In the cases where the CLRCEVs of the disclosure are used for diagnostic and/or theranostic purposes (e.g. in vivo imaging), a “pharmaceutically effective amount”, “effective amount”, and/or “diagnostically effective amount”, is intended to encompass an amount which provides a detectable signal for diagnostic and/or theranostic purposes. For example, the CLRCEVs of the disclosure may comprise agents capable of being detected by a detection means, which may allow for the detection and/or staging of a cancer or another disease or condition.

The term “purified,” as used herein means altered, isolated, or removed from the natural state. For example, a cell, a vesicle derived from a cell or any other cell fragment naturally present in vivo in a living animal does not qualify as “purified.” However, the same cell, vesicle, or cell fragment, either partially or entirely isolated from naturally coexisting substances/materials does qualify as “purified.” For example, red cells sourced from a cell bank are “purified” for at least two reasons: (1) packed red cells have been removed from “whole blood”, which generally contains both the cellular and the plasma components; and (2) packed red cells are generally provided in a leukoreduced form (i.e. white blood cells (or “white cells” or leukocytes) have been mostly or entirely removed). In terms of relative purity, the entire cellular fraction would be considered purified (with respect to whole blood) and leukoreduced packed red cells would be considered ever “more purified” (with respect to the cellular fraction of whole blood).

A purified red cell, CLRC, RCEV or CLRCEV composition can be present in a substantially pure form, or it can be part of a non-native milieu such as, for example, a culture medium. For example, in the case where lab-cultured red cells are used to produce the CLRCs of the disclosure, the red cell precursor cells would be present in pure form because they had been originally separated from a mixture of cells found in vivo (e.g. isolating CD34⁺ hematopoietic stem cells from human whole blood renders them purified).

A “source red cell,” as used herein, refers to a red cell or red cell precursor from which a red cell extracellular vesicle (RCEV) is derived. For example, it is envisioned that all red cells, including reticulocytes (in any stage of development) and mature red blood cells (i.e. lacking nuclei in mammals) may be loaded with cargo to form cargo loaded red cells (CLRCs). A “loaded source red cell” thus refers to a cargo loaded red cell source cell (CLRCSC) that may be vesiculated according to the disclosure to produce CLRCEVs.

The terms “exogenous agent” and “exogenous cargo” as used herein, refer respectively to an agent and cargo that: (1) do not naturally exist, or (2) are not naturally present in naturally occurring source red cells.

The terms “endogenous agent” and “endogenous cargo” as used herein, refer respectively to an agent and cargo that is already present when the source red cells are formed, irrespective of whether the red cells developed naturally in vivo or if the red cells derive from modified or unmodified progenitor cells.

The term “extracellular vesicle” (or “EV”) as used herein refers to a submicron vesicle-like structure released from a cell into the extracellular environment. EVs are substantially spherical/spheroid fragments of plasma or endosomal membrane between about 50 and about 1000 nm in diameter. Larger vesicles may be produced during several biological processes, for example, during red cell senescence. EVs are released from many different cell types, both in vitro (e.g. production of engineered exosomes from culture cells) and in vivo. EVs are membranous and may comprise, for example, a single membrane layer or a lipid bilayer. The EV membrane may originate from the plasma membrane or intracellular membranes, and hence, the EV may comprise a substantially similar composition as compared to the cell from which it is derived. In cases where the EV is made from a completely enucleated, mature mammalian red blood cell, said EV may derive entirely from the plasma membrane of the parental or source cell.

As used herein, EVs encompass apoptotic bodies, blebs, ectosomes, exosomes, membranous micro particles & nano particles and micro-/nanovesicles, and they may be classified into one of the foregoing based upon size and origin. Moreover, EVs may be produced via a variety of processes, including mechanical manipulations, outward budding and fission. As such, EV production may be artificial (e.g. chemical, mechanical, combinations thereof), natural or some combination of artificial and natural (e.g. chemical added to increase the natural secretion of exosomes from cultured cells).

Microvesicles or microparticles (or nanovesicles or nanoparticles) are generally produced by outward budding and are typically larger than exosomes, having diameters between about 100-500 nm. In some embodiments, EVs comprises microvesicles having diameters from about 50-1000 nm, 101-1000 nm, 101-750 nm, 101-500 nm, or 100-300 nm, or 101-300 nm. The EVs may be substantially uniform in size, having an average diameter of about 100, 110, 120, 130, 140, 150, 160, 170, 180, 190, 200, 210, 220, 230, 240, 250, 260, 270, 280, 290 or about 300 nm. In some embodiments, the EVs have an average diameter of about 130-150 nm or about 140 nm and a polydispersity index (POI) of between about 0.04 and 0.10 nm, 0.05 and 0.09 nm, 0.06 and 0.08 nm, or around 0.07 nm.

In contrast, exosomes tend to range from about 30-150 nm and are produced by many different cell types, both in vivo and in vitro. Several companies have built their platforms around the engineering, production and use of exosomes to deliver therapeutic cargo (e.g. US 2019/0151456 to Codiak). In further contrast, apoptotic bodies (or blebs) are larger still, having diameters of about 1-5 μm.

As discussed above, the invention is based in part upon the surprising finding that red cells previously loaded with a cargo may be subjected to vesiculation to produce a large quantify of high quality cargo loaded red cell extracellular vesicles (CLRCEVs). The Applicant initially demonstrated that red cells loaded with a cargo using Erytech's ERYCAPS® encapsulation platform could produce RCEVs having a clinically relevant amount of cargo retained therein (see FIG. 2). It was next determined that the number of RCEVs per cargo loaded red cell (CLRC) could be substantially increased by subjecting the ERYCAPS®-produced cargo loaded red cells (CLRCs) to subsequent treatments, including but not solely, mechanical and/or chemical vesiculation methods. As disclosed herein, such methods allowed the production of greater than 100 CLRCEVs from a single CLRC. In some embodiments, the subsequent vesiculation treatments may increase the number of clinically useful CLRCEVs by greater than about 50%, 60%, 70%, 80%, 90%, 100%, 200%, 300%, 400%, 500%, 600%, 700%, 800%, 900%, 1,000%, 1,500%, 2,000%, 2,500%, 3,000%, 5,000%, 10,000% or more.

In some embodiments, CLRCEVs are loaded with the same cargo. In other embodiments, CLRCEVs are loaded with two or more different types of cargo. Each CLRCEV may contain greater than one different cargo. In some cases, a plurality of the CLRCEVs contain one cargo, and another plurality of the CLRCEVs contain a different cargo. In some cases, the pluralities overlap, such that some CLRCEVs may be loaded with two or more different cargo molecules. In some embodiments, at least about 50%, 55%, 60%, 65%, 70%, 75%, 80%, 85%, 90%, 95%, at least about 97% or substantially all of the CLRCEVs encapsulate at least one cargo.

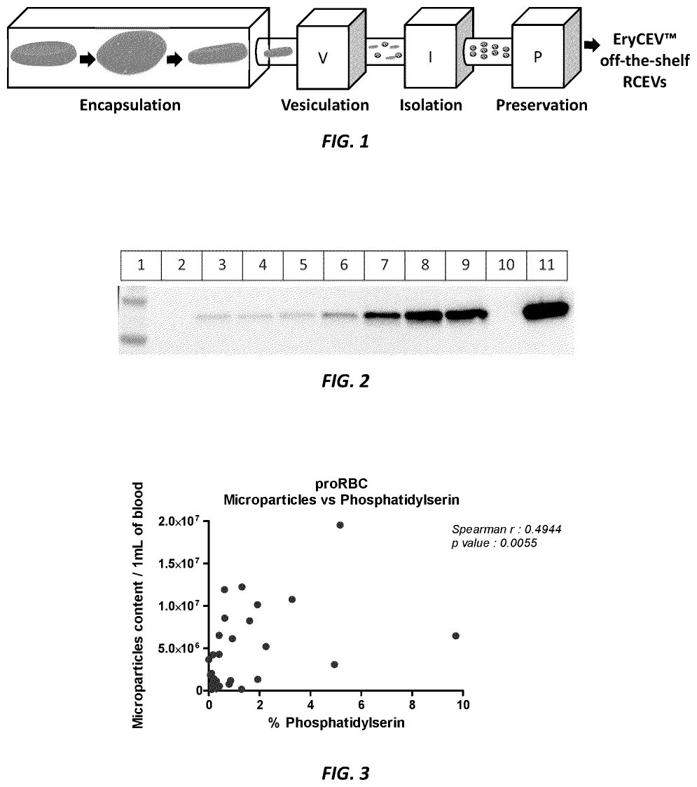

In some embodiments, CLRCEVs according to the disclosure may be produced by 1) loading red cells using any suitable encapsulation technology, including an hypotonic loading process, to produce CLRCs; 2) inducing vesicle formation (i.e. “vesiculation”) in the CLRCs; and 3) isolating and/or purifying CLRCEVs having desired characteristics. FIG. 1 provides a graphical representation of an EryCEV™ manufacturing platform according to the disclosure. As indicated, the platform may comprise a red cell encapsulation device, operably connected to a vesiculation device, operably connected to an isolation device, operably connected to a preservation device.

In some embodiments, desired CLRCEV characteristics may include an optimal amount of cargo per EV, an optimal diameter/size, and/or an optimal membrane topology. In other embodiments, the desired EV characteristics comprise membrane characteristics associated will relatively higher tumor retention times when said EVs are injected into a subject or patient in need of administration therewith. In still other embodiments, the desired EV characteristics include membrane characteristics associated with rapid clearance to reticuloendothelial system components including spleen, liver and/or bone marrow immune cells including phagocytic cells including macrophages. One advantageous red cell loading method is the ERYCAPS® encapsulation technology, disclosed in U.S. Pat. No. 8,617,840 (to Erytech).

In some embodiments, the platform may be completely automated (e.g. computer controlled), such that red cells and cargo(es) added to the loading device are automatically subjected to loading, vesiculation, isolation and then preservation (in that order), thereby producing ready-to-use, off-the-shelf EryCEV™ according to the disclosure. In some embodiments, inline manufacturing quality control measures may be incorporated into the platform to improve, for example, quality and yield. In some embodiments, the loading device comprises the ERYCAPS® device disclosed in the references cite herein.

In some embodiments, the vesiculation device comprises a means for subjecting loaded red cells to mechanical energy (e.g. as disclosed in the references cited herein). In particular, the vesiculation device may be capable of delivering an optimal amount of energy to the loaded red cells to maximize any one or more or all of the following: (1) yield of CLRCEVs per loaded red cell; (2) average amount of cargo per CLRCEV; (3) speed of production of CLRCEVs from CLRCs; (4) uniformity of CLRCEV size as measured, for example, by a relatively low polydispersity index (PDI). In general, PDI reflects the broadness of a molecular weight distribution of a given species (here, CLRCEVs). The larger the PDI, the broader the distribution. As such, in some embodiments, the vesiculation device provides a relatively low PDI, equating to a narrow distribution of CLRCEV sizes. For example, the CLRCEVs may be deemed to have a relatively low PDI when less than about 5% or about 10% of the CLRCEVs deviate from the mean CLRCEV size by more than one standard deviation. In some embodiments, greater than about 95% of isolated CLRCEVs have a size within one standard deviation of their mean size. In some embodiments, the mean size of CLRCEVs is about 70, 80, 90, 100, 110, 120, 130, 140, 150, 160, 170, 180, 190, 200, 210, 220, 230, 240, or 250 nm in diameter.

In some embodiments, the weight/weight (w/w) ratio of CLRCEVs produced during vesiculation to larger membranous material (e.g. non-completely vesiculated loaded red cells or large fragments thereof) may be about 10:90, 20:80, 30:70, 40:60, 50:50, 60:40, 70:30, 80:20, 90:10, 95:5, 97:3, 99:1 or even greater. In some embodiments, minimal isolation may be required post-vesiculation when the w/w ratio of CLRCEVs to other membranous material is very high (e.g. >99:1). That being said, the skilled artisan will appreciate that each device of the platform (i.e. encapsulation, vesiculation, isolation, and preservation devices) may comprise means for adapting to variable upstream device output. For example, the vesiculation device may comprise a controllable means for varying the intensity and duration of time that mechanical energy applied to red cells loaded by the encapsulation device. Likewise, the CLRCEV isolation component may comprise a means for adapting to varying ratios of CLRCEV to other membranous components. Applicant envisions that many other platform components and configurations may be routinely added now that the disclosure has been made.

In some embodiments, the isolation device comprises a means for isolating the CLRCEVs from non-CLRCEV materials/components received from the encapsulation device. In some embodiments, the isolation means may comprise a means for providing cross-flow based filtration combined with molecular weight size exclusion (e.g. Capto Core, from Sigma-Aldrich). In some embodiments, the molecular weight cutoff may be adjusted to yield CLRCEVs that are no more than about 100, 110, 120, 130, 140, 150, 160, 170, 180, 190, 200, 210, 220, 230, 240 or 250 nm in diameter. In some embodiments, the isolation means comprises multiple filtration components, each of which may be used, washed, and re-used as many times as required to isolate one or more batch of CLRCEVs. In some embodiments, the isolation means may comprise centrifugation components, in addition to or instead of size exclusion/filtration components. In some embodiments, the isolation device comprises a means for selecting for CLRCEVs having at least about 100, 200, 300, 400, 500, 600, 700, 800, 900, 1,000, 1,500, 2,000, 5,000 or more cargo units per EV.

In other embodiments, the red cells may be loaded using any suitable loading/encapsulation technology, including those available now and those yet to be developed. Such approaches may include the use of endocytosis, electroporation, mechanical squeezing (e.g. US 2018/0201889 to SQZ Biotechnologies), other microfluidic and/or mechanical means (e.g. US 2018/0155669 to Indee; WO2019018497 to Harvard; US 2019/0071695 to Cellino; and WO 2019/123372 to Politecnico di Milano), cell penetrating peptide, osmotic-based encapsulation, and variations and/or combinations thereof. Osmotic approaches may include hypotonic dilution, hypotonic pre-swelling, hypotonic dialysis (e.g. Erytech's ERYCAPS®) and osmotic pulse/isotonic osmotic lysis, genetic approaches (e.g. US 2018/0135012 to Rubius), via cell-penetrating peptide (CPP), or lipid nanoparticle (e.g. WO 2019/040516 to Rubius).

Compositions Comprising CLRCEVs and/or Additional Active Ingredients.

In some embodiments, it may be desirable to load the red cells in such that they remain substantially similar to native red cells. In some embodiments, the similarities comprise substantially similar metabolic capacity, surface presentation of phospholipids including PS, membrane rigidity/deformability, protein content, surface protein content, etc.

It is a further object of the disclosure to provide combinations of biologically active CLRCEVs and other active ingredients to produce safe and synergistically effective combinations for use in treating a subject or patient in need thereof. In some embodiments, the subject or patient is suffering from cancer and the treatment comprises simultaneously or sequentially administering synergistically effective amounts CLRCEVs and additional anticancer agents.

In some embodiments, the additional anticancer agents may be selected from one or more of the following: modified or unmodified enzymes (e.g. for depleting nutrients needed by certain cancer cells); encapsulated enzymes (e.g. ERYASPASE™, produced by Erytech, which is L-asparaginase encapsulated in red cells); immune checkpoint modulators including immune checkpoint inhibitor (ICI); antibody drug conjugates (ADC); chemotherapeutic agents (e.g. gemcitabine, 5-FU, platinum-based agents, taxanes, etc.); CAR T cells, CAR M cells, anticancer vaccines, immune modulator including immune adjuvants, etc.

Determination of a synergistic interaction between a CLRCEV and an additional anticancer agent may be based on the results obtained from the assays described herein. The results of these assays may be analyzed using the Chou and Talalay combination method and Dose-Effect Analysis with CalcuSyn software in order to obtain a Combination Index (Chou and Talalay, Trends Pharmacol. Sci. 4: 450-454; Chou, T. C. (2006) Pharmacological Reviews 68(3):621-681; Chou and Talalay, 1984, Adv. Enzyme Regul. 22: 27-55).

In some embodiments, the synergistically efficacy of combinations may be analyzed utilizing a standard program for quantifying synergism, additivism, and antagonism among anticancer agents. An exemplary program utilized is described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 to 1.2 indicate additive effects. The combination therapy may provide “synergy” and prove “synergistic”, i.e., the effect achieved when the active ingredients used together is greater than the sum of the effects that results from using the compounds separately. A “synergistic effect” may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes. In general, during alternation therapy, an effective dosage of each active ingredient is administered sequentially, i.e., serially, whereas in combination therapy, effective dosages of two or more active ingredients are administered together.

The person skilled in the art may understand from the present disclosure that the duration of treatment with one of the more than one active ingredient, and the delay between (for example) Active 1 and Active 2 treatment, may vary depending on the treatment, on the patient response and importantly on the half-life of the Active. There may also be a difference depending on the dosage form used in the invention, for example the differences between the following: a free enzyme, a pegylated enzyme, erythrocytes encapsulating an enzyme, EVs encapsulating an enzyme or enzyme bound to microcapsules (e.g. made of PLA or PLGA) or other types of particles including nanoparticles.

The disclosed compositions may be administered to a mammal using standard techniques. Techniques and formulations generally may be found in Remington's Pharmaceutical Sciences, 18^{th} ed., Mack Publishing Co., Easton, Pa., 1990 (hereby incorporated by reference).

Pharmaceutically acceptable carriers and/or excipients can also be incorporated into a pharmaceutical composition according to the invention to facilitate administration of the particular methioninase or asparaginase. Examples of carriers suitable for use in the practice of the invention include calcium carbonate, calcium phosphate, various sugars including lactose, glucose, or sucrose, or types of starch, cellulose derivatives, gelatin, vegetable oils, polyethylene glycols, and physiologically compatible solvents. Examples of physiologically compatible solvents include sterile solutions of water for injection (WFI), saline solution and dextrose.

Pharmaceutical compositions according to the invention can be administered by different routes, including intravenous (e.g. injection or infusion), intraperitoneal, subcutaneous, intramuscular, oral, topical (transdermal), or transmucosal administration. For systemic administration, oral administration may be used. For oral administration, for example, the compounds can be formulated into conventional oral dosage forms such as capsules, tablets, and liquid preparations such as syrups, elixirs, and concentrated drops.

Alternatively, injection (parenteral administration) may be used, e.g. intramuscular, intratumoral, intravenous (including infusion), intraperitoneal, and subcutaneous injection. For injection, pharmaceutical compositions may be formulated in liquid solutions, preferably in physiologically compatible buffers or solutions, such as saline solution, Hank's solution, Ringer's solution, or any solution suitable for the injection of red cells, e.g. red cell carrier solutions disclosed in U.S. Pat. No. 8,617,840 (to Erytech) or US 2016/0051482 (to Erydel). In addition, the EVs may be formulated in solid form and redissolved or suspended immediately prior to use. For example, lyophilized forms of the EVs (and/or additional anticancer agent) can be used.

For some additional active ingredients, systemic administration may also be accomplished by transmucosal or transdermal means. For transmucosal or transdermal administration, penetrants appropriate to the barrier to be permeated are used in the formulation. In addition, detergents may be used to facilitate permeation. Transmucosal administration, for example, may be through nasal sprays, inhalers (for pulmonary delivery), rectal suppositories, or vaginal suppositories. For topical administration, compounds can be formulated into ointments, salves, gels, or creams, as is well known in the art.

The invention also encompasses the use of implanted devices to deliver the CLRCEVs or additional active ingredient, for instance through infusion or another route. In a particular embodiment, the device comprises two chambers or vials, one containing CLRCEVs, the other containing a second anticancer agent. The device has, for each chamber or vial, a tube, and the like for delivering the active ingredient into the blood circulation, an electronic or electrical valve or pump, or an actuated piston, that may be controlled by an electronic circuit and a suitable software. The electronic circuit and its software controls the delivery of CLRCEVs and/or second active ingredient.

In an embodiment, an active ingredient is encapsulated inside CLRCEVs and the composition comprises a suspension of these CLRCEVs in a pharmaceutically acceptable carrier or vehicle.

“Encapsulated” means that the active ingredient is contained inside the EVs, with the further understanding that a small proportion of the active ingredient may remain associated with the cell membrane.

Cargo Loaded Extracellular Vesicles (CLRCEVs) from Loaded Red Cells

According to an embodiment, the composition comprises RCEVs encapsulating at least one cargo, which may include an API and a pharmaceutically acceptable vehicle. In some embodiments, the RCEVs are produced from loaded red cells or loaded red cell precursor cells (e.g. taken from a mammal of the same species as the treated subject or patient). When the mammal is a human, the red cells may be human red cells. In an embodiment, the red cells come directly from the subject or patient to be administered the CLRCEVs (i.e. loaded autologous RCEVs).

According to an embodiment, the pharmaceutically acceptable vehicle is a “preservation solution” for RCEVs (i.e. a solution in which the RCEVs encapsulating the cargo are suspended in their suitable form for being stored while awaiting their injection). A preservation solution may comprise at least one agent that promotes the preservation of the EVs (e.g. glucose, dextrose, adenine, or mannitol). The preservation solution may be an aqueous solution comprising NaCl, adenine and at least one compound from among glucose, dextrose, and mannitol. The preservation solution may comprise NaCl, adenine and dextrose, preferably an AS3 medium (see D'Amici et al. Blood Transfus. 2012 May; 10(Suppl 2): s46-s54, which is herein incorporated by reference in its entirety). The preservation solution may also comprise NaCl, adenine, glucose, and mannitol, advantageously a SAG-Mannitol (SAGM) or ADsol medium.

In particular, the composition or suspension, in a preservation solution, may be characterized by an extracellular hemoglobin (Hb) level maintained at a level equal to or less than 0.5, in particular 0.3, notably 0.2, advantageously 0.15, or even more advantageously 0.1 g/dl at 72 h and preservation at a temperature comprised between about 2 and about 8° C. The extracellular Hb level may be measured by the manual reference method described in G. B. Blakney and A. J. Dinwoodie, Clin. Biochem. 8, 96-102, 1975, or by any other suitable manual or automated method.

In other embodiments, the CLRCEVs may be produced in such a way that extracellular Hb is not a concern (i.e. there is little to no intra-vesicular Hb present after the CLRCEVs have been isolated).

### Methods of Encapsulation

Red cells may be encapsulated with a host of active ingredients using a wide range of technical approaches, including at least the following (and techniques yet to be developed): hypotonic loading (see WO 2006/016247 and WO 2017/114966, both to Erytech; US 2016/0051482 to Erydel; and WO 2013/045885, to St. Georges Hospital Medical School), mechanical/microfluidic loading (see US 2018/0201889, to SQZ; WO 2016/109864, to Indee, Inc.; WO 2019/018497, to Harvard), “soluporation” (see US 2017/0356011, US 2019/0194691, and US 2019/0217315, to Avectas), laser-assisted cell loading (see US 2019/0071695, to Cellino Biotech, Inc.), cell-penetrating peptide (CPP), electroporation, transfection and genetic expression (see WO 2016/183482 A1 to Rubius). All of the foregoing references are incorporated herein by reference in their entireties.

When hypotonic loading (also referred to as “lysis-resealing”) is used, red cells are exposed to hypotonic conditions to open pores in their membranes to allow active ingredients to enter the cells. Thereafter, the cargo loaded red cells (CLRCs) are resealed by exposing them to hypertonic conditions. At least three methods are routinely used: hypotonic dialysis, hypotonic preswelling and hypotonic dilution.

In hypotonic dialysis, a suspension of CLRCs may be obtained using the following method:

1—suspending red cells in an isotonic solution at a hematocrit level equal to or greater than about 40%, 45%, 50%, 55%, 60% or about 65%, cooling between about +1 and about +8° C.;

2—subjecting the red cells to a lysis procedure, at a temperature maintained between about +1 and about +8° C., comprising the passing of the suspension of red cells and cooled hypotonic lysis solution between about +1 and about +8° C., into a dialysis device (e.g. a coil or a dialysis cartridge);

3—subjecting the red cells to an encapsulation procedure by adding the cargo to be encapsulated into the suspension before or during lysis, at a temperature maintained between about +1 and about +8° C.; and

4-subjecting the red cells to a resealing procedure conducted in the presence of an isotonic or hypertonic, advantageously isotonic solution, at a higher temperature, notably comprised between about +25 and about +47° C. or between about +30 and about +42° C.

And while Erytech Pharma currently encapsulates type-matched, allogeneic, donated red cells, other red cells, including autologous or even xenogeneic red cells, may be loaded using the above process. Moreover, red cells produced using cell culture may also be used to practice any of the embodiments of this disclosure.

In some embodiments, the lysis-resealing methods described in WO 2006/016247 and WO 2017/114966 (both to Erytech and incorporated herein by reference) may be used to produce the CLRCs. In some embodiments, the hypertonic resealing step may be replaced with an isotonic resealing step, particularly in cases where a higher and/or more rapid yield of CLRCEVs is desired.

### Methods of Use

In another aspect, the invention comprises a method for treating a disease or disorder (e.g. a cancer) in a mammal in need thereof, the method comprising administering at least one dose of an effective amount of CLRCEVs according to the disclosure.

In some embodiments, the method comprises administering, especially injecting or infusing, to the mammal in need thereof, a composition comprising CLRCEVs.

### Targeting

In some embodiments, the RCEVs or CLRCEVs may be engineered and/or otherwise modified (e.g. via covalent conjugation of the parental red cells, as disclosed in US 2017/0326213, to the Augusta University Research Institute) to have peptides or antibodies that bind with some degree specificity (including substantially complete or complete) to target cell. Such surface modified RCEVs may be prepared according to the following steps: (1) providing cargo loaded red cells (CLRCs); 2) covalently conjugating to the surface of the CLRCs peptide(s) or protein(s) that have been activated in a reaction with N-hydroxysuccinimide (NHS) or N-hydroxysulfosuccinimide (sulfo-NHS) in the presence of 1-ethyl-3(3-dimethylaminopropyl)carbodiimide (EDC) or dicyclohexylcarbodiimide (DCC); and (3) vesiculating the surface modified CLRCs, thereby preparing the surface modified CLRCEVs.

In other embodiments, RCEVs may also be coated with antibodies fused to a C1C2 domain of lactadherin that bind to phosphatidylserine (PS) on the surface of red cells and RCEVs produced therefrom. RCEVs may also be contacted with targeting motifs that bind specifically to components of red cells (see e.g. US 2019/0382479 to EPFL, incorporated herein by reference).

In still other embodiments, surface modified RCEVs and CLRCEVs may be produced by beginning with surface modified RCs derived from lab-grown red cell precursor cells. Various methods are routinely used by the skilled artisan to engineer and grow red cells in the lab, notably including the following: Hawksworth J. et al., EMBO Molecular Medicine 10: e8454, 2018; US 2018/0135012 (to Rubius); US 2019/0249143 & US 2019/0290695 (to University of Rochester); U.S. Pat. No. 10,273,455 (to Hanyang University); US 2018/0291344 (to Boston University); and US 2019/0093080, U.S. Pat. No. 10,260,038, WO 2019/040649 & U.S. Pat. No. 10,471,099 (to Whitehead Institute Biomedical Research, WIBR), the disclosures of which are incorporated herein by reference in their entireties.

In some embodiments, the red cells may be thermally or chemically modified, so as to promote targeting of the resulting RCEVs. Such targeting may include relatively increased and/or enhanced phagocytosis of said RCEVs, in particular, by dendritic cells, which may be desired for vaccine formulations comprising CLRCEVs loaded with antigen and/or adjuvant. In some embodiments, heat treatment may be carried out under the following conditions: heating of RBCs for about 15 minutes to about 90 minutes, preferably from about 25 to about 50 minutes, at a temperature of between about 42 and about 55° C., preferably between about 47 and about 51° C. Typically, red cells may be heated for about 30 minutes at between about 48 and about 50° C., for example at about 48° C. In some embodiments, chemical treatment may be carried out using agents which modify the surface of the red cells, and in particular bridging or crosslinking agents such as bis(sulphosuccinimidyl) suberate (BS3 or BS3), glutaraldehyde and neuraminidase. In some embodiments, at least two methods of targeting may be combined.

### Enzyme Cargoes-Metabolic Diseases/Disorders and Cancer Indications

In some embodiments, red cells are loaded with cargo as disclosed in US 2010/0316620 (to Erytech). The loaded red cells are then vesiculated to produce CLRCEVs loaded with enzyme, which are suitable for treating Gaucher's disease. In some embodiments, the CLRCEVs comprise a glucocerebrosidase and are administered to subjects and patients in need thereof to introduce glucocerebrosidase into the lysosomal compartment of macrophages and/or Gaucher cells in the patient or subject's bone marrow. In some embodiments, the CLRCEVs comprise membrane topology associated with preferential targeting of macrophages and/or Gaucher cells in the bone marrow.

In some embodiments, red cells are loaded with cargo as disclosed in US 2016/0120956 (to Erytech). The red cells are then vesiculated to produce CLRCEVs loaded with a phenylalanine reducing enzyme (e.g. phenylalanine ammonia lyase (PAL) or phenylalanine hydroxylase (PAH)), which are suitable for treating phenylketonuria. In some embodiments, the CLRCEVs comprise a PAL or PAH and are administered to subjects and patients in need thereof to reduce the plasma/blood levels of phenylalanine. In some embodiments, the CLRCEVs comprise membrane topology associated with extended CLRCEV in vivo half-life, which leads to sustained reductions of plasma/blood phenylalanine.

In some embodiments, red cells may be loaded with cargo as disclosed in WO 2019/042628 (to Erytech). The red cells are then vesiculated to produce CLRCEVs loaded with an enzyme capable of reducing arginine levels (e.g. arginine deiminase or arginase), which are suitable for treating arginase-1 deficiency. In some embodiments, the CLRCEVs comprise an arginine deiminase or an arginase and are administered to subjects and patients in need thereof to reduce the plasma/blood levels of arginine. In some embodiments, the CLRCEVs comprise membrane topology associated with extended CLRCEV in vivo half-life, which leads to sustained reductions of plasma/blood phenylalanine. In other embodiments, the membrane topology of the CLRCEVs is associated with liver targeting, which provides enhanced reduction of arginine levels in the liver.

In some embodiments, red cells may be loaded with cargo as disclosed in U.S. Pat. Nos. 9,968,663, 8,974,802 10,046,009, US 2019/0000941, or US 2016/0120956 (each to Erytech and herein incorporated by reference in its entirety). The red cells are then vesiculated to produce CLRCEVs loaded with an enzyme capable of reducing the level of a metabolite. In some embodiments, the enzyme may reduce the level of one or more of the following: 2,8-dihydroxyadenine, isoleucine, argininosuccinic acid, pentose, (succinyl/2′-deoxy)adenosine, kynurenine, beta-alanine, phosphoenolpyruvate, ammonia, leucine, fructose, porphobilinogen, arginine, methionine, fumarate, proline, asparagine, oxalate, glyceraldehyde, pyruvate, carbon dioxide, phenylalanine, glyceric acid, serine, (gluco/galacto)cerebrosides, threonine, glycine, sphingosines, citrulline, thymidine, (hypo)xanthine, succinate semialdehyde, cysteine/cystine, tryptophan, lactate, uridine monophosphate, galactose-1-phosphate, tyrosine, lysine, glutamine, urea, nitric oxide, histidine, uric acid, ornithine, homocysteine, valine or orotic acid.

MicroRNA and LncRNA Cargoes (e.g. miRNA Replacement Therapy “MRT” for Cancer Indications)

MicroRNAs (“miRNAs”) are non-coding RNAs that post-transcriptionally regulate gene expression. Alterations in the expression of oncogenic or tumor suppressor miRNAs have been observed in connection with various the progression of certain diseases, including cancer. Moreover, the expression of some miRNA changes in response to anticancer therapies. As such, modulating the level of a specific miRNA may sensitize, or render vulnerable, a given cancer to specific anticancer agent.

In some embodiments, a miRNA is a nucleic acid having near or 100% identity to a target gene. In some embodiments, the miRNA may inhibit gene expression by hybridizing to a complementary cellular mRNA, thereby impairing expression of said mRNA. In some embodiments, the miRNA may be at least about 15-50 nucleotides in length. In some embodiments, the miRNA may comprise modified nucleotides to improve its characteristics, including half-life.

In some embodiments, it is envisioned that CLRCEVs may contain any miRNA cargo whose in vivo downregulation leads to and/or promotes diseases and disorders. CLRCEVs containing miRNA cargo may be administered to subjects or patients as an effective “microRNA replacement therapy” (MRT). See e.g. Mollaei H et al. J Cell Physiol., 2019 August; 234(8):12369-12384. “MicroRNA replacement therapy in cancer”, the disclosure of which is incorporated herein by reference.

And while MRT systemic miRNA delivery has previously led to unwanted or unacceptable immune responses and degradation by RNAses, it is envisioned that the RCEVs will protect the MRT cargos from such natural shocks. This outcome is reasonably expected in view of the thousands of doses of red cells encapsulated with an active ingredient have been safely administered to human patients as of this filing.

In some embodiments, the cargo is miR-34a and the resulting MRT-CLRCEVs are useful for inhibiting prostate tumor growth and bone metastasis.

In some embodiments, the cargo is one that normally targets KRAS, but is downregulated in certain cancers, for example pancreatic cancer. In such embodiments, the cargo may include miR-96, miR-217 (tumor suppressor), miR-216, miR-193b (inhibits KRAS, Akt, and ERK), miR-126, miR-143/145 (miRNA-RAS-associated feed-forward mechanism), let-7, miR-181a and miR-206. In some embodiments, CLRCEVs comprising any of the foregoing miRNAs may be combined with ASNase, METase and/or GLNase, each enzyme optionally encapsulated in red cells or in RCEVs.

In some embodiments, anticancer miRNA cargoes may include, for example, a member of the let-7, miR-34 or miR-125 family.

In some embodiments, compositions comprising CLRCEVs loaded with miRNA (“MRT-CLRCEVs”) may re-sensitize resistant tumor cells to anticancer agents when administered to a subject or patient in need thereof.

### Antigen Cargoes-Immune Modulation

In some embodiments, the CLRCEVs may be loaded with antigens and/or adjuvants, for use in eliciting in a subject or patient either an immune or tolerance response. For example, red cells loaded with antigens using Erytech's ERYCAPS® system are capable of eliciting a vaccine immune response (e.g. U.S. Pat. No. 9,950,049 to Erytech Pharma) or an immune tolerance response (e.g. US20120207745 to Erytech Pharma), depending upon certain factors including the presence or absence of an immune stimulant/adjuvant.

Now that the invention has been disclosed, Applicant envisions that when antigen-loaded red cells are subjected to vesiculation, the resulting antigen-loaded RCEVs will be able to elicit a safe and effective immune or tolerance response. In some embodiments, e.g. when an immune response is desired, immune stimulant or adjuvant is added to the CLRCEVs (and/or to the pharmaceutical compositions comprising the CLRCEVs) to stimulate a robust, safe, and effective immune response against the antigen cargo, and therefore against cells expressing the antigen or fragments or variants thereof). In other embodiments, e.g. when a tolerance response is desired, immune stimulants or adjuvants are specifically excluded from the CLRCEVs or pharmaceutical compositions comprising same.

In some embodiments, a tolerance response elicited by the administration of antigen-loaded RCEVs may be associated with any one, or more, or all of the following (BM=bone marrow; DC=dendritic cell):
(a) rapid blood clearance of the RCEVs (e.g. >50% EVs cleared within 1-24 hours of administration), and increased liver and/or spleen targeting. “Targeting,” as used herein encompasses increased or enhanced association of RCEVs and CLRCEVs with the indicated cell, tissue, or body location;
(b) relatively higher phagocytosis by liver & spleen DCs and macrophages including: liver CD11b+ cells, spleen CD11c+ DCs, F4/80 and CD11b+ cells, CD11c+ B220+ liver DCs;
(c) increases in the percentage of regulatory CD4+ T cells expressing FOXP3, regulatory CD4+CD25+ T cells producing IL-10, spleen CD4+ FOXP3 cells and liver CD4+ FOXP3 cells;
(d) increases in inhibitory CTLA-4 in CD4+CD25+ regulatory T cells;
(e) decreases in the proliferation/activation of antigen-specific spleen CD8+ T cells & T cell activity and α-antigen specific IgG Ab titer in sera, IFNγ production by antigen-stimulated splenocytes;
(f) increases in the percentage of spleen CD4+ FOXP3 cells and inhibitory CTLA-4 in spleen CD4+ CD25+ regulatory T cells;
(g) reduced spleen DC maturation markers and α-antigen specific CD8+ T cell activity.

In other embodiments, an immune response elicited by the administration of antigen-loaded RCEVs may be associated with any one, or more, or all of the following, particularly when the RCEVs are administered with an adjuvant, for example, P(I:C) (a TLR3 agonist) or CL-097 (a TLR7/8 agonist):

(a) increases in the percentage, number, and proliferative capacity of antigen-specific CD4+ T cells and in CD44 expression in CD45.2+CD4+ T cells;

(b) increases in IFNγ expression in splenocytes, percentage of antigen-specific CD8+ T cells, percentage of CD8+ T producing IFNγ and expressing CD107;

(c) decreases in the growth of antigen-expressing tumor cells;

(d) increases in phagocytosis by spleen F4/80 & CD11b macrophages, mDCs and pDCs;

(e) increases in the percentage of antigen-specific CD8+ T cells, percentage of CD62L+ and/or IFNγ cells in antigen-specific CD8+ T cell population and percentage of FOXP3+ in antigen-specific CD4+ T cells.

The CLRCEVs according to the invention may comprise one or more immune modulators. In some embodiments, the composition comprises at least two populations of CLRCEVs, each encapsulating a different immune modulator. Various types of immune modulators, including adjuvants can be used to stimulate immune cells: bacterial or viral RNA or DNA, heat shock proteins (HSPs), sugars, immune complexes and cytokines, and activators of Toll-like receptors (TLRs) and STING. Macrophages, dendritic cells, and other phagocytic immune cells do not all express the same receptors at their surface. As such, the choice of the immune modulator/adjuvant may vary depending upon the cell or cell type to be activated.

According to other embodiments, the adjuvant may be provided as a separate composition, that can be administered simultaneously with or separately from the CLRCEVs containing the antigen.

Immune modulators useful in the practice of the invention generally include any damage-associated molecular patterns (DAMPs) and/or pathogen-associated molecular patterns (PAMPs) that activate the innate immune cells including: TLRs, nucleotide-binding oligomerization domain (NOD)-like receptors (NLRs), absent in melanoma 2 (AIM2)-like receptors (ALRs), retinoic acid-inducible gene I (RIG-1)-like receptors (RLRs) or C-type lectin receptors (CLRs). DAMPs and PAMPs initiate a cascade of immune responses resulting in a potent innate and adaptive immune responses against cancers and pathogens. For additional discussion as regards the anti-tumor properties of TLRs, RLRs and STING, see L I et al. 2017 (International J. Molecular Sciences). And to focus on TLR signaling, ZHAO et al. provide a useful review (Front. Immunol., 23 Jul. 2014)

In some embodiments, immune modulators/adjuvants may include the following:

TLR ligands: including imidazoquinolones, including: imidazoquinoline, e.g. imidazoquinoline CL097, imiquimod, resiquimod; CpG oligodeoxynucleotides; LPSs (lipopolysaccharides); poly(inosinic acid)-(polycytidylic acid poly(I:C); HILTONOL® (poly-ICLC; by Oncovir), polyIC and IC-31. TLR ligands may include those disclosed in: US 2018/0169224 A1 (to WARF) and US 2006/0089326 A1 (to the University of Iowa Research Foundation), for example. HILTONOL® is an improved form of poly(I:C) adjuvant (activates TLR3), has been used in 90+ clinical trials (Ott et al., 2017, Nature), and exhibits marked synergy with αPD-1, αPD-L1, OX40, CD40, FLT3, CD27, 4-1BB and others.

STING ligands: which include natural and synthetic cyclic dinucleotides (CDNs), which are agonists of the “stimulator of interferon genes” (STING) protein. Activation of the STING pathway promotes TANK-binding kinase 1 (TBK1) signaling and activates NF-kB and IFN regulatory factor 3 (IRF3) in immune cells in the tumor microenvironment (TME). Activation of these pathways/events initiates production of pro-inflammatory cytokines including IFNs, including IFNβ, which augments cross-presentation of tumor-associated antigens (TAAs) by CD8α⁺ and CD103⁺ dendritic cells (DCs) to cytotoxic T lymphocytes (CTLs) (resulting ultimately in CTL-mediated tumor cell lysis). Ligands may include those disclosed in: U.S. Pat. No. 10,106,574 B2 (to Merck Sharp & Dohme), US 2018/0162899 A1 (to Janssen Biotech), US 2019/0031708 A1 (to IFM Therapeutics), US 2017/0158724 A1 (to GSK), US 2019/0016750 A1 (to Innate Tumor Immunity), etc.

Cytokines: interferon alpha, interleukin 2 (IL-2), interferon gamma (IFNγ), Granulocyte Monocyte-Colony Stimulating Factor (GM-CSF), interleukin 12 (IL-12), Tumor Necrosis Factor alpha (TNFα).

Bacterial constituents: BCG (Bacillus Calmette Guerin), MDP (Muramyl dipeptide), TDM (Trehalose dimycolate), LPS (lipopolysaccharide), MPL (monophosphoryl lipid A).

Mineral adjuvants: Al(OH)₃, AlPO₄, KPO₄, Ca(H₂PO₄)₂, etc.

Bacterial toxins: CT (cholera toxin from Vibrio cholera), CTB (cholera toxin, V. cholera), PT (pertussis toxin, B. pertussis) LT (thermolabile lymphotoxin from E. coli).

KLH: Keyhole limpet hemocyanin.

An object of the present invention is also an anti-tumor therapeutic comprising an effective amount of one or more immune modulating CLRCEV composition according to the invention. In some embodiments, the immune modulating CLRCEV composition may be administered simultaneously or sequentially with a composition comprising RCEV comprising or encapsulating one or more tumor antigens. In some embodiments, both the immune modulator and the tumor antigen may be encapsulated into the same RCEV.

The immune modulating compositions of the invention may be used in a method for inducing, in a subject or patient, a cytotoxic cellular response against tumor cells or a tumor. This method comprises administration to the subject or patient an effective amount of a CLRCEV composition according to the invention, e.g. intravenously, by injection or infusion. In particular embodiments, the method induces DC activation/maturation and CD8⁺ cytotoxic cellular responses. In some embodiments, the immune modulating erythrocyte compositions elicit a specific CD4⁺ helper and CD8⁺ cytotoxic response.

The compositions and vaccines of the invention may be used in an anticancer treatment method for inducing, in a patient, a cytotoxic cellular response as described above, against tumor cells or a tumor. This method comprises the administration to this patient of an effective amount of an immune modulating CLRCEV composition according to the invention, e.g. intravenously, by injection or infusion. Immune modulating compositions may comprise about 1 to about 50 mL of a suspension of CLRCEVS.

An object of the invention is also the use of a composition according to the invention for the manufacture of an immune modulating CLRCEV composition, comprising CLRCEVs containing intravesicularly at least one immune modulator.

The compositions of the invention may be used for inducing, in a host, a cytotoxic cellular response mediated by phagocytic cells, and directed against tumor cells or a tumor.

In some embodiments, the immune modulator may be a dendritic cell (DC) targeting and/or activating agent. In some embodiments, the immune modulator may be CpG or variants thereto. In some embodiments, the immune modulator may be selected from a TLR agonist, CPG, polyIC, poly-ICLC, 1018 ISS, aluminum salts, Amplivax, AS15, BCG, CP-870, 893, CpG7909, CyaA, dSLIM, flavone acetic acid (FAA), GM-CSF, IC30, IC31, Imiquimod, ImuFact, IMP321, IS Patch, ISS, ISCOMATRIX, Juvlmmune, LipoVac, MF59, monophosphoryl lipid A, Montanide IMS 1312, Montanide ISA 206, Montanide ISA 50-7, Montanide ISA-51, OK-432, OM-174, OM-197-MP-EC, ONTAK, PepTel®, imiquimod, resiquimod, gardiquimod, 3M-052, SRL172, YF-17D, VEGF trap, beta-glucan, Pam3Cys, Aquila's QS21 stimulon, vadimezan, and AsA404 (5,6-dimethylxanthenone-4-acetic acid, or “DMXAA”).

In some embodiments, TLR agonists may include natural and synthetic TLR ligands and several classes of small molecules including guanosine analogs (e.g. loxoribine and isatoribine), deaza-adenosine analogs and imidazoquinolines.

In some embodiments, CLRCEVs may comprise activators of TLR3, which elicit production of type I IFNs and NF-κB activation.

In some embodiments, CLRCEVs may comprise activators of TLR7, which lead to the induction of IFN-α and IFN-inducible cytokines including EP-10 and I-TAC. The levels of TLR7-induced IL-la/B, IL-6, IL-8, M1P-1α/β and MIP-3α/β may be lower than those induced by the activation of TLR8. TLR7 stimulation may lead to activation of innate immune cells (e.g. macrophages and natural killer (NK) cells).

In some embodiments, CLRCEVs may comprise activators of TLR8, which lead to the induction of pro-inflammatory cytokines (e.g. IFN-γ, IL-12p40/70, IL-1α/β, IL-6, IL-8, MIP-1α/β and MIP-3α/β, TNF-α). TLR8 agonists may be particularly effective in sustaining killer T cell-mediated responses as a consequence of increased IL-12 production. TLR8 stimulation may also activate APCs to stimulate adaptive immunity, which may enhance viral clearance and inhibit the growth of neoplasm.

In some embodiments, CLRCEVs may comprise activators of TLR9, which elicit the production and/or secretion of IFN-γ and IL-12.

In some embodiments, CLRCEVS comprising TLR7 and/or TLR9 agonists may be particularly useful in enhancing the immune response of a subject or patient against a tumor antigen. In some embodiments, said tumor antigens are administered along with the TLR-CLRCEVs as part of an anticancer therapeutic regimen. In other embodiments, the TLR-CLRCEVs enhance or adjuvant the patient's or subject's immune response against tumor antigens already present in vivo (e.g. tumor antigens including neoantigens). This utility is envisioned because type I interferons induced by activation of TLR7 and TLR9 are known to contribute to Th1-like humoral and cellular immune responses.

Accordingly, in some embodiments, when the immune modulating CLRCEV compositions are administered to a subject or patient, these compositions may effectively serve as an adjuvant for antigens already present in the subject or patient. In such embodiments, the immune modulating composition activates the immune system such that it responds (or once more responds, in the case of “cold” tumors) to cancer-cell-specific antigens within a subject's or patient's cancer/tumor. The compositions of this disclosure thus have the ability to awaken an immune system that has become suppressed or dormant as a consequence of “immunological dampening” associated with the growing cancer (e.g. the appearance of “don't eat me” signals on tumor cells, upregulation of adenosine receptors, etc.).

TABLE 1
IMMOD-CLRCEVs cargoes may include one or more of the following:

| Agent to Combine with RBC | Receptors | Cancer Types | References |
|---|---|---|---|
| Bacillus Calmette-Guérin (BCG) | TLR2/4 | Bladder cancer | [1] |
| monophosphoryl lipid A(MPL) | TLR4 | Cervical cancer | [2] |
| Imiquimod | TLR7 | Breast cancer | [3] |
| Flagellin-derived CBLB502 (Entolimod) | TLR5 | Hepatoma | [4] |
| 852A | TLR7 | Hematologic malignancy | [5] |
| CpG ODN | TLR9 | Glioblastoma | [6] |
| poly(I:C)/poly-ICLC | TLR3 | Multiple cancer types | [7] |
| 5′ ppp-siRNA for Bcl-2 | RIG-I | Melanoma | [8] |
| 5′ ppp-siRNA for TGF-β | RIG-I | Pancreatic cancer | [9] |
| HVJ-E | RIG-I | Prostate cancer, gliomas | [10, 11] |
| poly(I:C) | MDA5 | Ovarian cancer, Pancreatic cancer | [12, 13] |
| cGAMP | STING | Colon cancer | [14] |
| c-di-GMP | STING | Melanoma | [15] |
| STINGVAX | STING | Melanoma | [16] |

Open in a separate window
poly(I:C): polyinosinic-polycytidylic acid; CpG ODN: CpG-containing oligodeoxynucleotides; Bcl-2: B-cell lymphoma-2; TGF-β: transforming growth factor beta; HVJ-E: hemagglutinating virus of Japan envelope; cGAMP: cyclic AMP-GMP; RIG-I: retinoic acid-inducible gene-I; siRNA: small interfering RNA.
References
1. Herr H. W., Morales A. J. Urol. 2008; 179: 53-56.
2. Srivastava A. K. et al. Cancer Res. 2014; 74: 6441-6451.
3. Adams S. et al. Clin. Cancer Res. 2012; 18: 6748-6757.
4. Burdelya L. G. et al. PNAS USA. 2013; 110: E1857-E1866.
5. Weigel B. J. et al. Am. J. Hematol. 2012; 87: 953-956.
6. Carpentier A. et al. Neuro Oncol. 2010; 12: 401-408.
7. Ammi R. et al. Pharmacol. Ther. 2015; 146: 120-131.
8. Poeck H. et al. Nat. Med. 2008; 14: 1256-1263.
9. Ellermeier J. et al. Cancer Res. 2013; 73: 1709-1720.
10. Matsushima-Miyagi T. et al. Cancer Res. 2012; 18: 6271-6283.
11. Matsuda M. et al. J. Neurooncol. 2011; 103: 19-31.
12. Kubler K. et al. Eur. J. Immunol. 2011; 41: 3028-3039.
13. Bhoopathi P. et al. Cancer Res. 2014; 74: 6224-6235.
14. Li T. et al. Sci. Rep. 2016; 6: 19049.
15. Nakamura T. et al. J. Control. Release. 2015; 216: 149-157.
16. Fu J. et al. Sci. Transl. Med. 2015; 7: 283ra252.
17. Shi M. et al. Medicine (Baltimore) 2016; 95: e3951.

### Antigen Cargoes—Infectious Disease Indications

### In Vivo Induction of Antibody Against Coronaviruses in Mice

A polynucleotide encoding a coronavirus spike protein, or an immunologically effective variant or fragment thereof, is loaded into red cells. The red cells are vesiculated as described herein, and the resulting antigen-loaded RCEVs are formulated into a pharmaceutically acceptable composition for administration to a subject or patient in need thereof. Such RCEVs may be referred to herein as “SPIKE-CLRCEVs”. The composition may comprise, for example, saline or any pharmaceutically acceptable carriers, vehicles and/or excipients known to skilled artisans.

In some embodiments, the RCEVs comprise mRNA encoding a coronavirus spike protein and are modified to target dendritic cells. In some embodiments, the membrane topology of the RCEVs naturally lead to the accumulation of said RCEVs in dendritic cells. In some other embodiments, the RCEVs further comprise adjuvant for activating dendritic cells and/or other immune cells.

SPIKE-CLRCEVs may be injected via any suitable route, for example intradermal, intramuscular, intravenous, or subcutaneous. In some embodiments, an immunostimulatory agent (or a polynucleotide encoding an immunostimulatory agent) may be concomitantly administered with the SPIKE-CLRCEVs to elicit an enhanced immune response against the SPIKE protein. When RCEVs contain an immunostimulant including an adjuvant, they are referred to herein as “STIM-CLRCEVs”. In some embodiments, a suspension of SPIKE-CLRCEVs and STIM-CLRCEVs are administered to elicit protective immunity against subsequent challenge with a virulent coronavirus. RCEVs encapsulating at least one SPIKE component and one STIM component are referred to herein as SPIKE-STIM-CLRCEVs. In some embodiments, such SPIKE-STIM-CLRCEVs are highly effectively in eliciting protective immunity in a subject or patient in need thereof.

In some embodiments, the SPIKE protein has a sequence as disclosed in Comput Biol Med. 2020 April 11:103749, “COVID-19 Coronavirus spike protein analysis for synthetic vaccines, a peptidomimetic antagonist, and therapeutic drugs, and analysis of a proposed Achilles' heel conserved region to minimize probability of escape mutations and drug resistance.” Any other SPIKE proteins (or sequences encoding same) may be encapsulated to produce SPIKE-CLRCEVs according to this disclosure. For example, the SPIKE protein may be encoded by any of the following Gen Bank sequences: QJA17276.1, QIK50427.1 and QIZ16509.1.

In some embodiments, the SPIKE protein may have a sequence having at least about 80, 90, 95, 98 or more identity to a sequence having the sequence as set forth in accession number QJA17276.1, QIK50427.1, QIZ16509.1 or, CAD0240757.1.

In some embodiments, subjects and patients may receive both a prime and a boost administration of an effective amount of SPIKE-CLRCEVs and/or SPIKE-STIM-CLRCEVs. Boosts may be administered, for example, weekly, bi-weekly, tri-weekly, or monthly until a desired level of virus-neutralizing antibodies is achieved, as measured for example by a suitable ELISA test.

The efficacy of vaccines comprising SPIKE-CLRCEVs and/or SPIKE-STIM-CLRCEVs may be rapidly evaluated using any suitable coronavirus model system. In some embodiments, the model system is a genetically engineered model (GEM), for example: transgenic mice expressing human ACE2; Ace2 Knockout mice; Tmprss2 Knockout mice; or Stat1 Knockout mice. In other embodiments, standard mouse strains may be used, in particular, strains adapted to coronaviruses, in particular, COVID-19. In other embodiments, vaccine efficacy may be evaluated in mice comprising a humanized immune system. For example, immunodeficient NOG mice engrafted with human peripheral blood mononuclear cells (e.g. huPBMC-NOG) have been used to study vaccine responses against SARS.

### Small Molecule Cargoes

In some embodiments, red cells are loaded with cargo as disclosed in US 2014/0363413 (to Erytech). The loaded red cells are then vesiculated to produce CLRCEVs loaded with a small molecule pharmaceutical agent. In some embodiments, the CLRCEVs comprise a small molecule that may have unacceptable toxicity when administered to a subject or a patient in its free (i.e. unencapsulated) form. In some embodiments, the CLRCEVs comprise membrane topology associated with preferential targeting.

### Other Indications & Applications

Extracellular vesicles originating from host and parasite are released throughout malaria infection. For example, both exosomes and extracellular vesicles have been found in infected red cells (iRC), which may contain parasites/parasite material, induce gametocytogenesis, be pro-inflammatory, and mediate cell-cell communication between parasites. Moreover, host-derived vesicles and particles, originating from endothelial cells, monocytes, platelets, and red cells, have been implicated in disease severity and pathology (Sampaio N G et al., 2017. “The role of extracellular vesicles in malaria biology and pathogenesis”. Malar J 16(1):245).

In some embodiments, CLRCEVs according to the disclosure may encapsulate cargoes that are active against malaria parasites. In such embodiments, a parasite-killing effective amount of the CLRCEVs may be administered to a patient suffering from malaria. In some embodiments, the CLRCEVs are taken up by iRCs, wherein the parasiticidal cargoes are released to inactivate or otherwise disable the parasites. In some embodiments, CLRCEVs comprise targeting moieties (e.g. on their exterior membranes) that bind to components expressed on the surfaces of red cells infected with malaria parasites.

In other embodiments, red cells may be loaded with imaging agents using the ERYCAPS® process disclosed herein, or any other suitable means for encapsulating red cells. Once the red cells are loaded with a desired imaging agent, the CLRCs may be vesiculated as described herein. CLRCEVs loaded with imaging agents may have the advantage of being capable of penetrating deep into hypoxic areas (e.g. portions of solid tumors) and/or crossing the blood/brain barrier.

In still other embodiments, CLRCEVs may also be loaded with agents capable of improving vascular function and/or endothelial regeneration, for example, to treat cardiovascular disease.

Accordingly, in some embodiments, CLRCEVs according to the disclosure may improve the safety, efficacy and/or targeting of cargoes including biologics and small molecules. The application will now be described further in the following non-limiting Examples.

### EXAMPLES

### Example 1: Production of Cargo Loaded Red Cell Extracellular Vesicles (CLRCEVs) from Cargo Loaded Red Cells (CLRCs)

Prior to the present disclosure, it was not known whether red cells loaded using the ERYCAPS® platform could be vesiculated to produce loaded red cell extracellular vesicles. To determine whether this was possible, packed, leukoreduced, human red cells were sourced and loaded with a test protein (“PRO”) or FITC-dextran (“DEX”) using the ERYCAPS® platform, which is detailed herein and in U.S. Pat. No. 10,273,444 (to Erytech), to produce PRO-CLRCs and DEX-CLRCs, respectively. The DEX-CLRCs were then vesiculated using a process adapted from WO 2019/002608 (to the University of PARIS, CNRS, and GENETHON; also referred to herein as the “EVerZom process”), to produce DEX-CLRCEVs. FIG. 2 is a representative blot showing the presence of cargo in proRBC-derived CLRCEVs.

To produce DEX-CLRCEVs about 2 to about 10 mL of DEX-CLRCs suspended to a concentration of about 0.5 billion cells/mL were centrifuged at 1500-2000 g for 5 minutes. For a non-loaded control, ERYCAPS® processed red cells were also subjected to vesiculation. Aliquots of either DEX-CLRCs or processed RCs were subjected to the following conditions in spinner flasks:

1) High stress (very strong agitation);

2) Medium stress (strong agitation);

3) Control level of stress (weak agitation);

4) Control+vesiculation agent (calcium ionophore).

As disclosed in WO 2019/002608, “strong agitation” is accomplished when the agitator causes a flow characterized by a Kolmogorov length LK=35 pm. Likewise, “weak agitation” (also referred to as “weak stirring” conditions) corresponds to the agitator driving a flow characterized by a length LK=50 pm. Finally, “very strong” agitation corresponds to a length LK<about 25 pm.

Samples from each of the above vesiculation groups were then analyzed using Nanosight Tracking Analysis (NTA). Briefly, samples were initially centrifuged at 2,000×g to pellet the larger components (e.g. remaining portions of incompletely vesiculated DEX-CLRCs, or large fragments or portions thereof). EVs were further purified using ultracentrifugation (˜110,000×g). Pellets from both spins were then analyzed by NTA. The EVs may also be purified using size exclusion membranes and/or sucrose cushion/gradient centrifugation.

Briefly, the results indicated that large number of DEX-CLRCEVs could be produced by subjecting DEX-CLRCs to vesiculation. In general, strong agitation (group 2, medium stress) yielded the highest number of DEX-CLRCEVs (>about 30 EVs per red cell), although the skilled artisan may now optimize RCEV production by optimizing agitation conditions.

Accordingly, this study established a proof of concept that CLRCEVs may be produced by using mechanical agitation to vesiculate ERYCAPS®-loaded red cells.

### Example 2: Production of Cyclic Dinucleotide (CDN) Cargo Loaded Red Cell Extracellular Vesicles (CDN-CLRCEVs) from Cargo Loaded Red Cells (CLRCs)

Immune responses may be induced in a subject or patient to treat diseases and disorders such as infections and cancer. Such responses may be accomplished via activation of pathogen recognition receptors (PRRs) using pathogen-associated molecular patterns (PAMPs) molecules, which activate innate immune responses. PAM PS molecules used in the practice of the disclosed invention include, but are not limited to: bacterial lipopolysaccharides (LPSs; activate TLR4), stimulator of interferon genes (STING) agonists, bacterial flagellin (TLR5), peptidoglycan (TLR2), lipoteichoic acid (TLR2), nucleic acids/derivatives normally associated with viral agents including dsRNA (TLR3) or non-methylated CpG (TLR9).

Unfortunately, the field has struggled to develop safe and effective methods for delivering such immune activators for a variety of reasons. For one, some of these immune activators induce significant and unacceptable toxicity when administered to subjects or patients. In addition, activating PRRs has been challenging because it generally requires intracellular delivery of the corresponding agonists. And furthermore, cancers employ varied and sophisticated immune evasion strategies, which lead to insufficient efficacy of many clinical cancer therapies. To counteract tumor-induced immunosuppression, it may be advantageous to reactivate specific elements of a patient's immune system. Promising immune targets include, but are not limited to, RIG-I-like Receptors (RLRs), Stimulator of Interferon Genes (STING) and Toll-like Receptors (TLRs).

Applicants reasoned that combining an effective amount of one or more of these immune activators with red cell extracellular vesicles (RCEVs) could reactivate immune functionality that had been suppressed by the activity of tumor cells. Delivering immune modulators/activators using immune modulator cargo loaded RCEVs (IMMOD-CLRCEVs) provides a number of benefits, including reduced toxicity, improved trafficking to immune cells of the reticuloendothelial system (e.g. RES), increased efficacy, etc. In fact, as disclosed herein, IMMOD-CLRCEVs may be tagged or decorated with any number of molecules to facilitate their accumulation in desired areas, including the tumor microenvironment (TME) or the reticuloendothelial system (RES).

Part 1—Successful Encapsulation of CDN (STING Agonist) into Red Cells and Testing of CDN-Loaded Red Cells in a Mouse Colon Cancer Model.

A STING agonist was encapsulated in RBCs using the ERYCAPS® method as discussed herein. The red cell suspension was washed several times before being brought to a hematocrit of about 70% for the hypotonic dialysis. The dialysis was carried out for about 1 hour (or about 30 min when the dialysis occurred after a heat treatment). The red cells were then resealed by means of a solution of high tonicity (i.e. greater than about 300 mOsm) for about 30 minutes. After a few washes, the final product was resuspended in a buffer, Sag-mannitol, and the hematocrit was brought to about 50%.

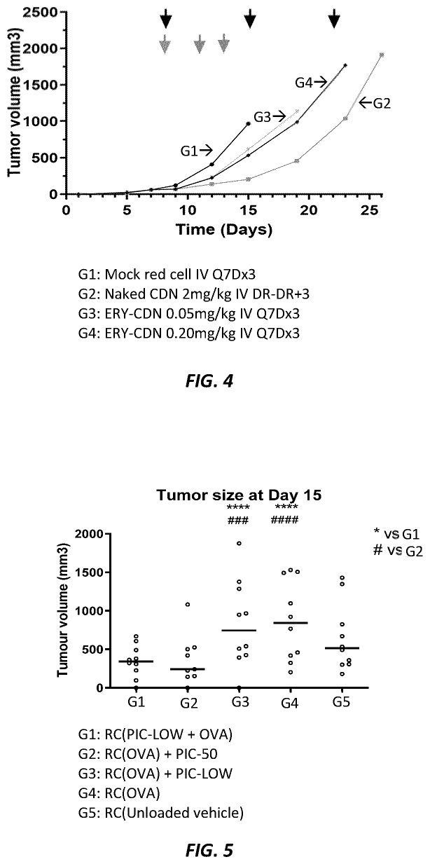

Briefly, CT-26 (ATCC® CRL-2638™) were seeded into 10 mice per group and CDN dosing was initiated when tumors grew to between about 100 and 150 mm³. Groups included: mock red cell (G1), non-encapsulated (naked) 2 mg/kg STING agonist (G2), red cell-encapsulated 0.05 mg/kg CDN STING agonist (G3), and red cell-encapsulated 0.2 mg/kg CDN (G4). Tumor volume was evaluated biweekly for 8 weeks and body weight was evaluated daily for the first week and biweekly thereafter. The Ery-CDN treatments did not induce significantly more weight loss than the naked CDN treatment. Based upon the tumor inhibitory efficacy of the red cell-encapsulated CDN (FIG. 4), it is estimated that red cell encapsulation significantly reduces the amount of CDN required to elicit an immune response comparable to nearly 10-fold more naked CDN. Likewise, survival was statistically similar between mice in G4 as compared to mice in G2, indicating that encapsulation of CDN into red cells may be dose-sparing.

Part 2—Successful Production of CDN-CLRCEV from the CDN-Loaded Red Cells of Part 1, and Testing Thereof in a Mouse Colon Cancer Model.

In view of the foregoing examples, it was hypothesized that red cells containing CDN could be vesiculated to produce an even more effective delivery vehicle for immune modulators like CDN STING agonists. Therefore, red cells are loaded with CDN as described above and the resulting CDN-CLRCs are vesiculated as disclosed. CT-26 (ATCC® CRL-2638®) are seeded into 10 mice per group and CDN dosing is initiated when tumors grew to between about 100 and 150 mm³. Groups include: mock CDN-CLRCEV (G1), non-encapsulated (naked) 2 mg/kg STING agonist (G2), 0.05 mg/kg CDN STING agonist delivered in CDN-CLRCEV (G3), 0.2 mg/kg STING agonist delivered in CDN-CLRCEV (G4), 0.05 mg/kg CDN STING agonist delivered in CDN-CLRC (G5), 0.2 mg/kg STING agonist delivered in CDN-CLRC (G6). Tumor volume is evaluated biweekly for 8 weeks and body weight is evaluated daily for the first week and biweekly thereafter. The Ery-CDN treatments do not induce significantly more weight loss than the naked CDN treatment. Based upon the superior tumor inhibitory efficacy of the CDN-CLRCEV as compared to both naked CDN and CDN-CLRC, it is estimated that encapsulation in CLRCEV significantly reduces the amount of CDN required to elicit an immune response superior to at least about 10-fold more naked CDN. Likewise, survival is statistically superior between mice in G4 as compared to mice in G2 (and also G5 & G6), indicating that encapsulation of CDN into red cells may be dose-sparing.

### Example 3: Production of TLR Ligand Cargo Loaded Red Cell Extracellular Vesicles (TLR-Ligand-CLRCEVs) from Cargo Loaded Red Cells (CLRCs)

Applicants reasoned that combining an effective amount of one or more of these immune activators with red cell extracellular vesicles (RCEVs) could adjuvant an immune response against a tumor-antigen, including antigens provided as part of a therapeutic regimen and, endogenous antigens including tumor antigens, including neoantigens.

Part 1—Successful Encapsulation of TLR Ligand into Red Cells and Testing of TLR Ligand-Loaded Red Cells in a Mouse EG7-OVA Tumoral Regression Model

A TLR3 agonist (Poly(I:C) or “PIC”) was encapsulated in red cells as described in Example 1. Mice were seeded with EG7-OVA and treated at days 3 and 7 post-seeding. Treatment groups included red cells encapsulating a low amount of P(IC) adjuvant plus OVA (OVA-PIC-high-RC, G1); OVA-RC plus separate administration of a P(IC)-high (G2); OVA-RC plus P(IC)-low (G3); OVA-RC (G4); unloaded RC (vehicle, G5). The “low” amount of PIC equated to about 1.6 μg, while the “high” amount of PIC was about 50 μg. As shown in FIG. 5, both the G1 and G2 treatments were significantly more effective than either the G3 or G4 treatment. In contrast, the G1 and G2 treatments did not produce significantly different tumor inhibition effects. This finding suggests that 1.6 μg TLR3 ligand co-encapsulated with antigen has substantially similar efficacy to about 50 μg unencapsulated TLR3 ligand. Accordingly, when co-loaded with an antigen, the TLR3 ligand-RC (i.e. Poly(I:C), MW<660 kDa) induced a clear and specific antigen immune response.

Part 2—Successful Production of TLR-Ligand-CLRCEV from the TLR-Ligand-Loaded Red Cells of Part 1, and Testing Thereof in a Mouse EG7-OVA Model

In view of the foregoing examples, it was hypothesized that red cells containing TLR ligand could be vesiculated to produce an even more effective delivery vehicle for immune modulators like TLR ligand. Therefore, red cells are loaded with TLR ligand as described above and the resulting TLR-ligand-CLRCs are vesiculated as disclosed. EG7-OVA cells (mouse thymoma cells) are seeded into 10 mice per group and TLR ligand dosing is initiated when tumors grow to between about 100 and 150 mm³. Groups may include: unloaded CLRCEV+OVA (G1); OVA+TLR-low (G2); OVA+TLR-high (G3); OVA-CLRCEV+TLR-low (G4); OVA-CLRCEV+TLR-high (G5); OVA-CLRCEV+TLR-high-CLRCEV (G6); OVA-CLRCEV+TLR-high-CLRCEV (G7); OVA-TLR-low-CLRCEV (G8); OVA-TLR-high-CLRCEV (G9). Tumor volume is evaluated biweekly for 8 weeks and body weight is evaluated daily for the first week and biweekly thereafter. In some embodiments, the co-encapsulated OVA-TLR-low-CLRCEVs are even more effective than red cells loaded with the same amount of antigen and TLR ligand.

### Example 4: Measuring Lipid Composition in RCEVs

In some embodiments, the lipid composition of RCEVs is substantially similar to the red cells from which they were derived. The lipid measurements may be produced using mass spectrometry and RCEVs and/or CLRCEVs are prepared as disclosed. Any suitable lipid analysis service provider (e.g. Dresden, Germany) may be used, e.g. as described (Sampaio, et al., PNAS, 2011, February 1; 108(5):1903-7). Lipids are extracted (Ejsing, et al., PNAS, 2009, March 17; 106(7):2136-41) and samples may be spiked with suitable lipid standard(s). RCEV lipid composition is then compared to that of parental red cells. In some embodiments, RCEVs and parental red cells will be deemed to have substantially similar lipid compositions when >80% of the lipids in the red cells are present in the RCEVs, and of those lipids, the level in the RCEV will be >50% of the corresponding lipid level in the parental cell. Lipid analyses of external and internal red cell and RCEV membrane leaflets may also be performed.

### Example 5: Measuring Proteomic Composition in RCEVs

In some embodiments, the protein composition of RCEVs will be similar to the red cells from which they are derived. RCEVs are prepared as described and resuspended in lysis buffer. Mixtures are then lysed, spun down and protein content is measured by a colorimetric assay. Samples are then generally prepared for characterization by LC-MS/LC-MS/MS. Standard procedures are performed and the resulting data is then analyzed to determine levels and proportions of known proteins. Molar ratios may be determined as the sum of the molar quantity of a given protein divided by the sum of the molar quantities of all identified proteins. In an embodiment, RCEVs and parental red cells are deemed to have a substantially similar proteomic composition when >80% of the identified proteins are present in the RCEV and in an amount of >50% of the corresponding protein level in the parental red cell.

### Example 6: Quantifying a Cargo Protein Level Per RCEV

This Example describes the quantification of cargo in RCEVs, which is generally expressed as a function of average cargo units (ACU) per RCEV. In an embodiment, RCEVs comprise a cargo that is originally loaded into parental red cells. The red cells may be loaded with cargo, or may be produced from precursor red cells that were themselves loaded with cargo or loaded with molecules encoding the cargo. In some embodiments, red cells may be loaded and/or grown using methods known to the skilled artisan. In some particular embodiments, the red cells are loaded using the ERYCAPS® hypotonic loading/resealing platform described herein. Thereafter, the cargo loaded red cells (CLRCs) may then be vesiculated using any suitable means, including those described and/or referenced herein. In some embodiments, the CLRCs are advantageously vesiculated using methods disclosed in WO 2019/002608 A1 (to the University of PARIS, CNRS, and GENETHON).

In an embodiment, e.g. when the RCEV cargo is a protein, cargo quantification may be accomplished by comparing protein levels in a plurality of RCEVs against a standard curve of protein concentration. For example, purified cargo protein may be serially diluted to generate a standard curve of protein concentration. Concentration of the standard curve and a sample of RCEVs may then be measured using a suitable method (e.g. Western Blot, ELISA, LC-MS, etc.) to calculate the average molar concentration of cargo protein molecules in the RCEVs. The molar concentration may then be converted to number of cargo protein molecules and divided by the number of RCEVs per sample to achieve an average number of cargo protein molecules per RCEV.

In an embodiment, the RCEVs will have at least about 1, 2, 3, 4, 5, 10, 20, 30, 40, 50, 60, 70, 80, 90, 100, 500, 10³, 3.0×10³, 5.0×10³, 10⁴, 5.0×10⁴, 10⁵, 3.0×10⁵, 5.0×10⁵, 10⁶, 5.0×10⁶, or more protein cargo molecules per RCEV.

### Example 7: Measuring the Potential of RCEVs to Target Cells in a Patient or Subject

This Example provides a method for evaluating the in vivo targeting potential of RCEVs prepared according to the disclosure. In some embodiments, the red cells used to produce the RCEVs contain no additional targeting motifs. In other embodiments, the red cells are modified to contain targeting motifs, which allow the ultimately produced RCEV to target a specific site, cell, or tissue in the body of a subject or patient.

Eight-week-old C57BL/6J mice are intravenously injected with RCEVs that express a marker such as luciferase. RCEVs are produced from red cells that either express marker, have been loaded to contain marker, or that do not contain marker (negative control). Mice are humanely euthanized hourly (from 1-12 hours) and then again at 24 hours after injection and tissues are evaluated for marker expression. In the case of luciferase, mice may receive via IP injection 150 mg/kg bioluminescent substrate 5 minutes prior to euthanization. After euthanization, brain, heart, intestine, kidney, liver, lungs, pancreas, spleen, and stomach are collected and imaged. Quantification is performed per routine methods, and the ratio of marker signal between target organs and non-target organs is calculated as a measure of targeting to the target organ.

In an embodiment, RCEVs and/or CLRCEVs target the liver more than any other organ. In some embodiments, more than about 30%, 35%, 40%, 45%, 50% or more of the RCEVs and/or CLRCEVs administered into an animal are taken up by cells of the liver, including immune cells present in the liver. In some embodiments, more than about 20%, 25%, 30%, 35%, 40%, 45%, 50% or more of the RCEVs and/or CLRCEVs are taken up by cells present in one or more cancerous growth. In some embodiments, greater than about 40% of the RCEVs and/or CLRCEVs are taken up by the liver and greater than about 20% are taken up by the intestine. In some embodiments, more than about 5% of the RCEVs and/or CLRCEVs are taken up by the stomach.

In some embodiments, CLRCEVs according to the disclosure containing CDN or other suitable anticancer cargo(es) may be particularly effective against cancers of the liver, intestine and/or stomach.

In some embodiments, CLRCEVs containing CDN or other anticancer cargo(es) reduce metastases that would otherwise develop in the liver, intestine and/or stomach.

In some embodiments, where targeting motifs are added to the red cells, the resulting RCEVs and/or CLRCEVs may target specific cells, such as cells expressing tumor-specific antigens, or cells expressing viral antigens.

### Example 8—CLRCEV-CDN Efficacy in an EMT-6 TNBC Syngeneic Mouse Model

Breast cancer is the most common cancer in women with 54,000 new cases diagnosed in France in 2015. Triple-negative breast cancers (TNBCs), a subtype defined by the absence of estrogen and progesterone receptors and the lack of HER2 overexpression (ER-PR-HER2−), tends to be more aggressive than other types. Chemotherapy is the primary established systemic treatment for patients with TNBC in both early and advanced-stages of the disease. The lack of targeted therapies and the poor prognosis of TNBC patients have fostered a major effort to discover safe and effective new therapies.

Study Aim. To Evaluate the Antitumor Activity of CDN-CLRCEV.

Briefly, mice bearing orthotopic EMT-6 syngeneic breast carcinoma mouse model are injected once weekly for 4 consecutive weeks with CDN-CLRCEV (various doses), unencapsulated/naked CDN or unloaded RCEV (control). Mouse body weight, as well as the length and width of the tumor, are measured twice a week. Tumors from animals receiving CDN-CLRCEV, CDN or control are collected throughout the study for size measurement, metabolite measurement, immunophenotyping and/or identification of biomarkers. Analysis of health parameters throughout the study reveal that all treatments are well tolerated by animals bearing the OT EMT-6 model. Several growth parameters are considered to evaluate the benefit of CDN-CLRCEV. For example, CDN-CLRCEV will elicit a greater delay before the tumor begins to grow exponentially and, an increase in animal survival, relative to either naked CDN or control.

Moreover, when EMT6 tumor cells are treated with increasing concentrations of CDN-CLRCEV, the expression of immune checkpoint molecules may increase (e.g. PD-1). Such cases may indicate that CDN-CLRCEV may be effectively combined with immune checkpoint inhibitors (e.g. anti-PD-1 antibodies) to exert synergistic effects against cancers including breast cancer. Similarly, CDN-CLRCEV may sensitize the tumors to other anticancer approaches, and such potential sensitization may be revealed by analyzing the tumor data collected and produced during this study. Accordingly, this is the first in vivo demonstration that CDN-CLRCEV is significantly more effective at impairing the growth of breast cancer tumors relative to unencapsulated CDN or control.

Additional protocol details. EMT-6 tumor cells (ATCC® CRL-2755™) are grown as a monolayer at 37° C. in a humidified atmosphere (5% CO2, 95% ambient air). The culture medium is RPMI 1640 containing 2 mM L-glutamine (ref: BE12-702F, Lonza) supplemented with 10% fetal bovine serum (ref: P30-1506, PAN). Tumor cells are detached from the culture flask by a 5-minute treatment with trypsin-versene (ref: 13E17-161E, Lonza), in Hanks' medium without calcium or magnesium (ref: BE10-543F, Lonza) and neutralized by addition of complete culture medium. The cells are counted in a hemocytometer and their viability assessed by 0.25% trypan blue exclusion assay.

One hundred twenty-two (122) healthy female BALB/c (BALB/cByJ) mice, 6-7 weeks old, are obtained from Charles River. The mice are maintained in SPF health status according to the relevant standards and housed according to the following: temperature: 22±2° C.; humidity 55±10%; photoperiod (12 h light/12 h dark); HEPA filtered air; 15 air exchanges per hour with no recirculation. Moreover, complete food is provided for immunocompetent rodents—R/M-H Extrudate is used during the acclimation period and at the start of study. It is then replaced by the A04 controlled standard diet (Safe®, France), which is used until the end of the study.

Induction of EMT-6 tumors in animals. The mice are anaesthetized with isoflurane and a 5 mm incision is made in the skin over the lateral thorax to expose mammary fat pad (MFP). About 2.5×10⁵ EMT-6 breast cells suspended in a volume of 50 μL RPMI 1640 medium are injected into the MFP tissue (right upper teat) by via tuberculin syringe taking care to avoid the subcutaneous space. After injection, the syringe is removed and the thoracic surface is gently dabbed with a 95% ethanol-dampened cotton-swab to kill tumor cells that may leak from the injection site. The day of injection is designated DO.

The treatment begins when the tumors reach a mean volume of between about 50-100 mm³. Eighty-six (86) out of the one hundred twelve (112) mice are randomized according to their individual tumor volume into eight (8) groups each of ten (10) or thirteen (13) animals using Vivo Manager® software (Biosystemes, Couternon, France). Treatments commence on randomization day “DR”.

Sample Collection. Twenty-four hours before the 1^{st} treatment and 24 hours after the last treatment, blood is collected by jugular vein puncture from all mice of groups 1-7 into blood collection tubes containing Lithium Heparin as anticoagulant. The tubes are immediately centrifuged at 1000 g for 10 minutes at +4° C. to obtain plasma. The plasma samples (1 tube per animal, 50 μL/tube) are stored in 1.5 mL propylene tubes at −80° C. until shipment (in cases where insufficient plasma is collected, the volume is adjusted to 50 μL with 0.9% NaCl, and appropriate notations are made). The maximum volume of blood that is collected is adjusted to the body weight of animals. As regards tumor collection, satellite mice from groups 2 and 5 (3 per group) are sacrificed around D15 so when tumor reach a volume of between about 500 and about 1000 mm³. Tumors are collected and cut into two parts that are weighed, snap-frozen and stored at −80° C. until analysis.

Clinical monitoring. All study data, including animal body weight measurements, tumor volume, clinical and mortality records, and treatment are scheduled and recorded on Vivo Manager® database (Biosystemes, Dijon, France). The viability and behavior are recorded every day and body weights are measured twice a week. The length and width of the tumor are measured twice a week with calipers and the volume of the tumor is estimated by the following formula: Tumor volume=(width²×length)/2. A tumor volume of 1000 mm³ is considered to be equal to 1 g. Humane endpoints are those known to the skilled artisan, including tumors exceeding 10% of normal body weight or exceeding 1500 mm³, tumors interfering with ambulation or nutrition, >8 mm ulcerated tumor, infection, bleeding, etc. Moreover, the following evaluation criteria of health are determined using Vivo Manager® software (Biosystemes, Couternon, France): individual and mean (or median) animal body weights; mean body weight change (MBWC): average weight change of treated animals in percent (weight at day B minus weight at day A divided by weight at day A). The intervals over which MBWC are calculated are chosen as a function of body weight curves and the days of body weight measurement.

Efficacy Assessment. The treatment efficacy is assessed in terms of the effects of the test substances on the tumor volumes of treated animals relative to control animals. The following evaluation criteria of antitumor efficacy are determined using Vivo Manager® (Biosystemes, Couternon, France):

1) individual and/or mean (or median) tumor volumes

2) tumor doubling time (DT)

3) tumor growth inhibition (T/C %) defined as the ratio of the median tumor volumes of treated versus control group, calculated as: T/C %=[(median tumor volume of vehicle treated group at DX)/(median tumor volume of treated group at DX)]*100. The optimal value is the minimal T/C % ratio reflecting the maximal tumor growth inhibition achieved. The effective criteria for the T/C % ratio according to NCI standards, is 42%

4) Relative tumor volume (RTV) curves of test and control groups are drawn. The RTV are calculated following the formula: RTV=(TV at DX)/(TV at DR), with DX: Day of measurement; DR: Day of randomization. Volume V and time to reach V. Volume V is defined as a target volume deduced from experimental data and chosen in exponential phase of tumor growth. For each tumor, the closest tumor volume to the target volume V is selected in tumor volume measurements. The value of this volume V and the time for the tumor to reach this volume are recorded. For each group, the mean of the tumor volumes V and the mean of the times to reach this volume are calculated.

Statistical Tests. All statistical analyses are performed using Vivo Manager® software (Biosystemes, Couternon, France). Statistical analysis of mean body weights, MBWC, mean tumor volumes at randomization, mean tumor volumes V, mean times to reach V and mean tumor doubling times are performed using ANantigen. Pairwise tests are performed using the Bonferroni/Dunn correction in case of significant ANantigen results. A p-value <0.05 are considered significant.

This study is repeated using candidate combination partners for the CDN-CLRCEVs. In some embodiments, asparaginase (ASNase), methioninase (METase) and/or glutaminase (GLNase) are usefully combined with CDN-CLRCEVs to exert synergistic efficacy against tumors including breast cancer tumors.

### Example 9—miRNA-CLRCEV Efficacy in an EMT-6 TNBC Syngeneic Mouse Model

CLRCEV comprising an anti-cancer miRNA are loaded into red blood cells and the resulting CLRCs are vesiculated to produce miRNA-CLRCEV according to the disclosure. Thereafter, the miRNA-CLRCEV are tested for efficacy according to the method described in Example 8.

### Example 10—Characterization of Cargo-Loaded Red Cell Extracellular Vesicles (CLRCEVs)

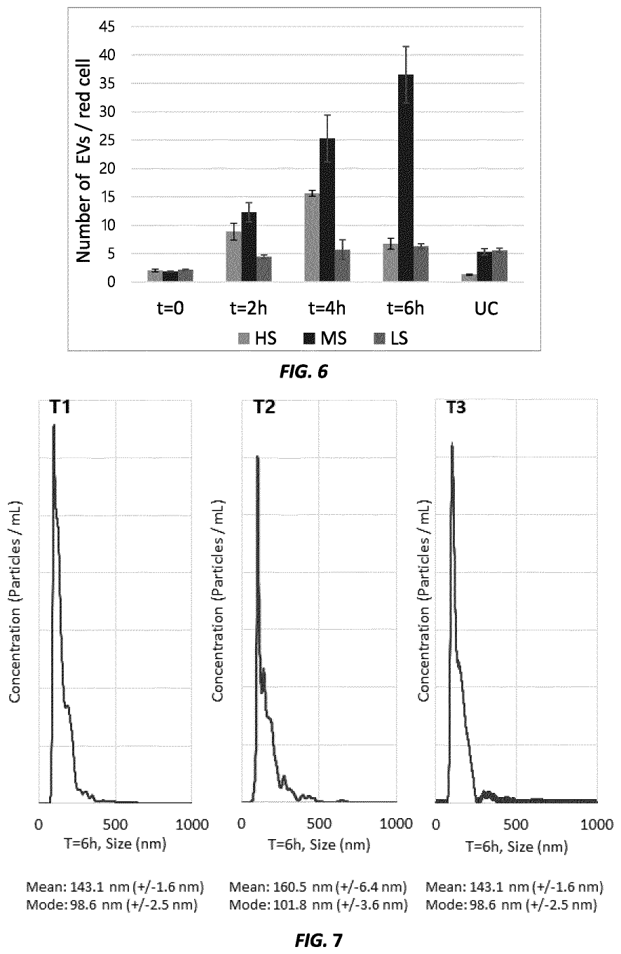

CLRCEVs containing FITC-dextran were produced using the methods disclosed in Example 1, except that the agitation parameters of the vesiculation step were varied to increase the yield of cargo-loaded red cell extracellular vesicles (CLRCEVs) per cargo-loaded red cell (CLRC). It was determined that the medium speed of agitation produced at least about 30 CLRCEVs per parental CLRC. For example, as demonstrated by the results in FIG. 6, six (6) hours of medium speed vesiculation performed on FITC-dextran-loaded red cells yielded superior numbers of CLRCEV/CLRC than either the low or fast speeds. Moreover, the six (6) hour time point provided superior vesiculation performance over the earlier time points, and it is envisioned that further vesiculation improvements may be achieved by optimization of the parameters disclosed herein.

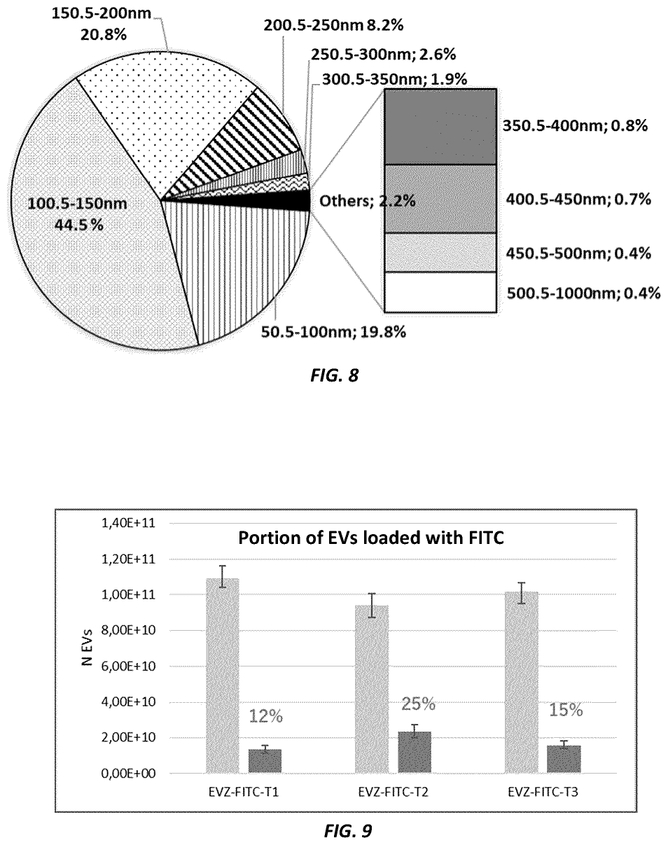

In more detail, three (3) batches of FITC-dextran CLRCEVs were produced and routinely characterized to determine the reproducibility of the MS agitation vesiculation method. NTA results are summarized in Table 2 and in FIGS. 7-9. Analysis of the mean size distribution indicated that about 85% of FITC-dextran-CLRCs had a diameter between about 50-200 nm (FIG. 8). As regards the cargo, the particle matrix analysis of these three batches (prior to purification) indicated that at least 17% of FITC-dextran CLRCEVs were contained a detectable amount of FITC (FIG. 9). After purification by ultracentrifugation (UC), the mean number of molecules per EV was estimated to be about 1.85E5 as indicated in Table 2. And after additional optimization, it was determined that a little less than medium speed (MS) and a concentration of about 0.5 million CLRCs per mL further improved vesiculation, resulting in greater than about 20% of the CLRC material being converted to CLRCEV material.

Moreover, in experiments where CLRCs were allowed to “age” for several days after having been loaded with FITC-dextran, up to about 170 CLRCEVs could be produced per CLRC. In view of these data, the inventors envision that “artificial aging” of CLRCs could achieve a similar enhancement in vesicle yield while avoiding degradation of either the vesicles or the encapsulated active components. Such aging could be accomplished by modifying the chemical composition of vesiculation medium, by applying an appropriate amount and duration of energy (e.g. heat, mechanical, electromagnetic, etc.) to the vesiculating red cells, or by combining both approaches.

TABLE 2
Characteristics of FITC-dextran CLRCEVs

| | T1 | | T2 | | T3 | |
|---|---|---|---|---|---|---|
| | Mean (SD) | | Mean (SD) | | Mean (SD) | |
| Mean size at 6 h | 145.9 nm | (±4.3) | 160.5 nm | (±6.4) | 143.1 nm | (±1.6) |
| Mean EVs/RBC | 50 | (±5) | 34.5 | (±3) | 40.9 | (±1) |
| Mean size after UC | 174.4 nm | (±3.4) | 181.6 nm | (±2.1) | 185.3 nm | (±1.0) |
| Mean EVs/RBC After UC | 3.0 | (±0.6) | 3.0 | (±0.6) | 6.8 | (±0.6) |

| | | | |
|---|---|---|---|
| EVs/ml | 1.06E11 | 1.3E11 | 1.24E11 |
| FITC Dextran [μg/ml] After UC | 9 | 17 | 14 |
| FITC Dextran molecules/ml | 1.39E16 | 1.26E16 | 2.17E16 |
| FITC Dextran molecules/EV | 1.32E5 | 2.55E5 | 1.75E5 |

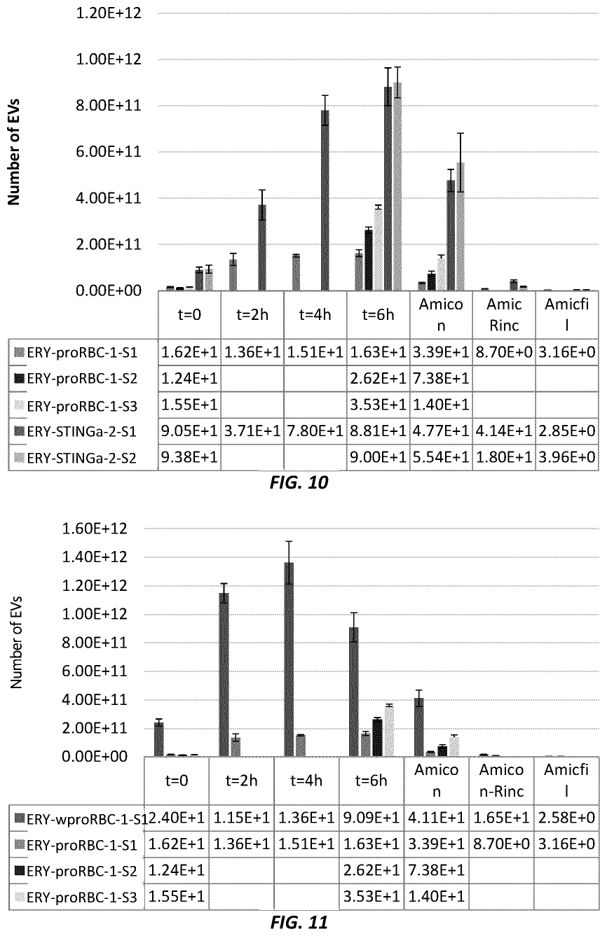

After the initial work using FITC-dextran, red cells were loaded with a STING agonist (ADU-S100, CAS No.: 1638241-89-0) and vesiculated as described in Example 2 above. FIG. 10 is a graph showing the total number of STINGa-CLRCEVs (batches ERY-STINGa-2-S1 and ERY-STINGa-2-S1) vs. the number of mock-loaded CLRCEVs (batches ERY-proRBC-1-S1, ERY-proRBC-1-S2 and ERY-proRBC-1-S3) produced by applying about 85% MS agitation to either STINGa-loaded or mock-loaded red cells for 0, 2, 4, or 6 hours. Red cells used for the production of STINGa-loaded and mock-loaded red cells came from the same human donor. Table 3 presents additional characteristics of STINGa-CLRCEVs produced at the T6 h time point, both before and after purification by Amicon filtration (AF) or ultracentrifugation (UC). Nanoparticle tracking analysis (NTA) indicated that about 40 EVs were produced per red cell at 6 hours, and that the mean size of the EVs had a range of about 158 to about 187 nm. After AF or UC purification, the yield was at least about 70% and it was also determined that AF performed at 3000 g for 30 min provided superior results compared with 2000 g for 30 min. Moreover, the yield of EVs per RBC at 6 h varied from about 20 to about 59 (total number at T6 h) and about 8.3 to about 53 (when the number of EVs present at T0 h was subtracted out).

TABLE 3
STINGa-CLRCEV production and characteristics

| ERY- | Mean | ERY- | | EVs/ | | | EV | | Mean |
|---|---|---|---|---|---|---|---|---|---|
| STINGa | ADU- | STINGa- | EVs/ | RBC | Mean | Mean | purifi- | Mean | ADU- |
| CLRC | S100 | EV | RBC | (T6H- | EVs/ml | size | cation | Purified | S100 |
| Batch | [μg/ml] | Batch | (T6H) | T0H) | (T6H) | (nm) | (yield) | EVs/ml | (μg/ml) |
| 1 | 672 | 1-S1 | 25 | 11.3 | 1.36E10 | 170.1 | UC (27%) | 1.05E11 | 9.26 |
| | | 1-S2 | 20 | 8.3 | 1.10E10 | 186.6 | AF (44%) | 9.61E10 | 12.8 |
| 2 | 788 | 2-S1 | 59 | 53.3 | 2.75E10 | 158 | AF (67%) | 1.69E12 | 38.1 |
| | | 2-S2 | 59 | 52.8 | 2.57E10 | 176.5 | AF (76%) | 7.59E11 | 30.2 |
| 3 | 578 | 3-S1 | 46 | 36 | 2.17E10 | 170.7 | AF (44%) | 7.19E11 | 12.1 |
| | | 3-S2 | 42 | 31 | 1.79E10 | 175.3 | AF (39%) | 8.68E11 | 10.7 |

Table 4 presents the characteristics of mock-loaded CLRCEVs produced by applying about 85% MS agitation to mock-loaded red cells (proRBC) for 6 hours. As in Table 3, EVs/RBC yield, mean size, EV mean concentration before purification, method and yield of purification, and EV mean concentration after purification are indicated. The letter “5” plus a number refers to a given production run or batch.

TABLE 4
Mock-loaded proRBC-EV production and characteristics

| Batch | Batch | | EVs/ | | | EVs | |
|---|---|---|---|---|---|---|---|
| ERY- | mock- | EVs/ | RBC | Mean | Mean | purification | Mean |
| pro- | loaded | RBC | (T6H- | EVs/ml | size | concentration | Purified |
| RBC | CLRCEVs | (T6H) | T0H) | (T6H) | (nm) | and (yield) | EVs/ml |
| 1 | 1-S1 | 10.3 | 9.3 | 5.21E9 | 158 | AF (33%) | 2.32E11 |
| | 1-S2 | 15.3 | 14.6 | 7.57E9 | 174.3 | AF (34%) | 1.08E11 |
| | 1-S3 | 24.5 | 23.4 | 1.02E10 | 167.4 | AF (48%) | 2.46E11 |
| 2 | 2-S1 | 8.7 | 6.5 | 4.75E9 | 193.3 | AF (43%) | 2.59E11 |
| | 2-S2 | 8.7 | 6.3 | 4.38E9 | 190.5 | AF (37%) | 1.95E11 |
| 3* | 3-S1 | 80.4 | 61.6 | 4.55E10 | 172.3 | AF (40%) | 1.25E12 |
| | 3-S2 | 59.0 | 43.1 | 3.09E10 | 182.1 | AF (43%) | 1.19E12 |

*vesiculation performed at D3 instead of D2 post red cell encapsulation

NTA results showed that the level of EVs at TO was very low at about 1 EV/RBC. Previously, t=0 was around a least 5 EVs/RBC. Further, the production of EVs from proRBC varied significantly, from about 10 to about 25 EVs/RBC. This number was significantly and substantially lower than the number of STINGa-EVs obtained from red cells previously loaded with STINGa (see above). Inventors envision that this difference may be caused by the absence of cargo in the mock-loaded proRBCs. Since red cells are known to shed undesirable components, cargo-loaded red cells (CLRCs) may produce more EVs per red cell than corresponding mock-loaded red cells, irrespective of the vesiculation means applied to the CLRCs. Therefore, a “control cargo” protocol is being developed to produce control EVs that are more similar to each given CLRCEV (e.g. an irrelevant small molecule-loaded CLRCEV serving as a control for a STINGa-loaded CLRCEV). Such control cargo may include, for example, FITC-dextran. The differences in initial EV yield notwithstanding, the size range of the mock-loaded EVs was similar to that observed for STINGa-EVs, but after AF, the final yield was relatively lower (between about 30% and 50%). As regards the AF purification, the majority of EVs were recovered during the first sampling, the second rinse contained less than 10% of the EVs, and no EVs were found in the filtrate. Finally, batches 3-51 and 3-S2 produced 3 days after encapsulation instead of 2 days after (as for the other batches) exhibited 2 to 10 times more EVs per red cell, demonstrating that age is an optimizable parameter for EV production.

Next, the above-described vesiculation techniques were applied to red cells that had been subjected to hypotonic encapsulation conditions but not to the final hypertonic resealing conditions (as was the case for the production of proRBC). Instead, after the hypotonic loading step, the red cells were directly returned to physiologic isotonic conditions (i.e. about 275 to about 300 mOsm). Red cells encapsulated in this manner are referred to throughout as “wproRBCs” and FIG. 11 presents a graph comparing the number of mock-loaded CLRCEVs made from wproRBCs vs. proRBCs. As before, mock-loaded red cells were subjected to about 85% of medium speed agitation for 0, 2, 4, or 6 hours, and purification by AF was performed. Red cells used for the production of both the wproRBC and proRBC came from the same human donor. EV production from wproRBC was up to 100 EVs/RBC, the EVs were of characteristic sizes, and UF yielded about 54%. The majority of EVs were recovered during the first sampling, the second rinse contained less than 10% of the EVs, and no EVs were in the filtrate.

Summarizing the above results, proRBCs produced lower and more varied amounts of EVs compared to either wproRBC or STINGa-loaded RBCs. As such, it is envisioned that vesiculation speed and/or magnitude can be further optimized by 1) exposing red cells to larger concentrations of active ingredient during the hypotonic encapsulation step; 2) allowing the cells to remain in contact with active ingredient and hypotonic solution for longer periods of time; and/or 3) not resealing the hypotonically encapsulated red cells in hypertonic buffer, but instead, placing them directly into isotonic buffer.

Accordingly, in some embodiments, the method for producing CLRCEVs from CLRCs comprises the following steps:

1) providing a plurality of red cells;

2) subjecting the red cells to hypotonic conditions;

3) contacting the red cells with active ingredient;

4) allowing the red cells and the active ingredient to remain in the hypotonic conditions for a sufficient period of time to promote a high percentage of loading (e.g. as evidenced by an about equal concentration of active ingredient both inside and outside of the red cells) and a subsequent high yield of CLRCEVs per CLRC (e.g. more than about 100, 110, 120, 130, 140, 150, 160, 170, 180, 190, 200, or more CLRCEVs per CLRC);

5) changing the hypotonic conditions to isotonic conditions (e.g. about 275 to about 300 mOsm) after the sufficient period of time;

6) not subjecting the cells to hypertonic conditions;

7) subjecting the loaded red cells (CLRCs) to a vesiculation means (e.g. mechanical energy, electromagnetic energy, vibrational energy, sound energy, and/or chemical means); thereby producing the CLRCEVs from the CLRCs.

### Example 11—In Vitro Evaluation of STINGa-CLRCEV Uptake by Monocytes and Macrophages

Phagocytosis experiments can be done with either THP1 monocytes (not differentiated) or THP1 macrophages (differentiated using PMA). THP-1 cells are widely used to elucidate macrophage responses to inflammatory stimuli, as well as the development and screening of potential therapeutics (Lund et al, 2016). Cells acquire a macrophage-like phenotype when they are differentiated using phorbol 12-myristate 13-acetate (PMA). Differentiated macrophages are the resident tissue phagocytes, serving as sentinel cells of the innate immune response (Daigneault M et a/l. 2010). Prior to this disclosure, there was no known standardized protocol for the reliable differentiation of THP-1 monocytes to a macrophage phenotype using PMA (Lund et al, 2016).

To test the ability of the RCEVs and CLRCEVs to be taken up by immune cells, we developed an in vitro phagocytosis protocol based upon the THP1 monocyte cell line. Briefly, we determined THP1 cells could be effectively differentiated to become more macrophage-like by treating them with PMA for 3 days followed by 4 days of resting in culture media without PMA. Thereafter, we applied various test substances (e.g. EVs-STINGa, EVs-proRBC, free STINGa, each+/=a reporter dye) to either the undifferentiated or differentiated THP1 cells and observed the phagocytosis activity.

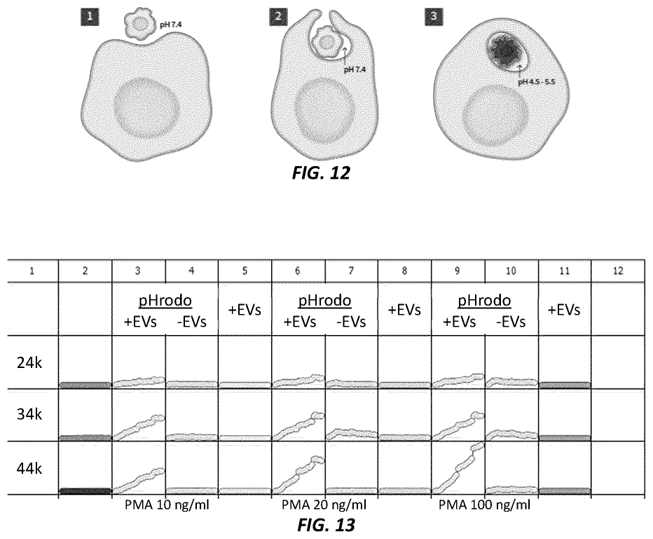

As shown schematically in FIG. 12, pHrodo®-labeled cells or vesicles (e.g. EVs) are added to phagocytic cells, but while they are outside of the cells, there is little to no fluorescence at the approximately pH 7.4 extracellular environment. When the relevant cellular receptors are engaged, a phagocytic cup forms around the labeled cells or vesicles. Finally, an acidic phagosome forms, causing the pHrodo® to emit fluorescence.

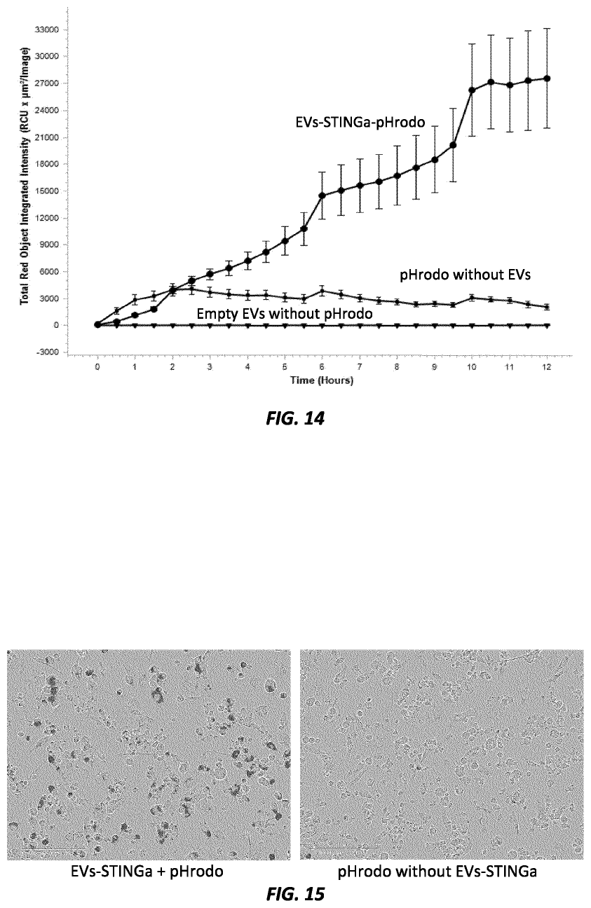

As an initial study, the THP1 cells were seeded as follows: 24,000, 34,000, and 44,000 cells/well. After seeding, three (3) concentration of phorbol-12-myristate-13-acetate (PMA) were tested for their ability to differentiate the THP1 cells into their macrophage-like state: 10, 20 and 100 ng/ml. Cells were incubated in their respective concentration of PMA for 72 h and thereafter the medium was replaced, and the cells were allowed to rest for 96 h in fresh media. Near the end of the resting time, 5000 EVs-STINGa/cell were labeled with pHrodo (1 μg/ml) for 1 h at 37° C. Labeled EVs-STINGa were added to differentiated THP1 cells and incubated for 12 h and the INCUCYTE device was set to read the wells every 30 minutes. As shown in FIG. 13, seeding of a larger number of THP1 cells resulted in increased phagocytosis as indicated by increased fluorescence (total red object integrated intensity) over twelve (12) hours. The highest red fluorescence intensity was observed for cells differentiated with 100 ng/ml of PMA. A dose effect based on the number of cells was observed. In a subsequent experiment, cells were again differentiated with 100 ng/mL PMA, seeded at a concentration of about 50k cells/well, and treated with labeled EVs-STINGa or negative controls (pHrodo®+EVs or EVs alone). As shown in FIG. 14, EVs-STINGa+pHrodo yielded increasing amounts of fluorescence over time, but pHrodo alone only yielded a small baseline amount of fluorescence during the study. These findings are supported visually by the micrographs of FIG. 15, which show the uptake by THP1-derived macrophages of EVs-STINGa+pHrodo (left) but not pHrodo alone (right). In these greyscale micrograms, the original red fluorescence appears black inside the cells. Taken together, these results indicate that the overwhelming majority of red fluorescence is attributable to the phagocytosis of EVs labeled with pHrodo, not free pHrodo alone.

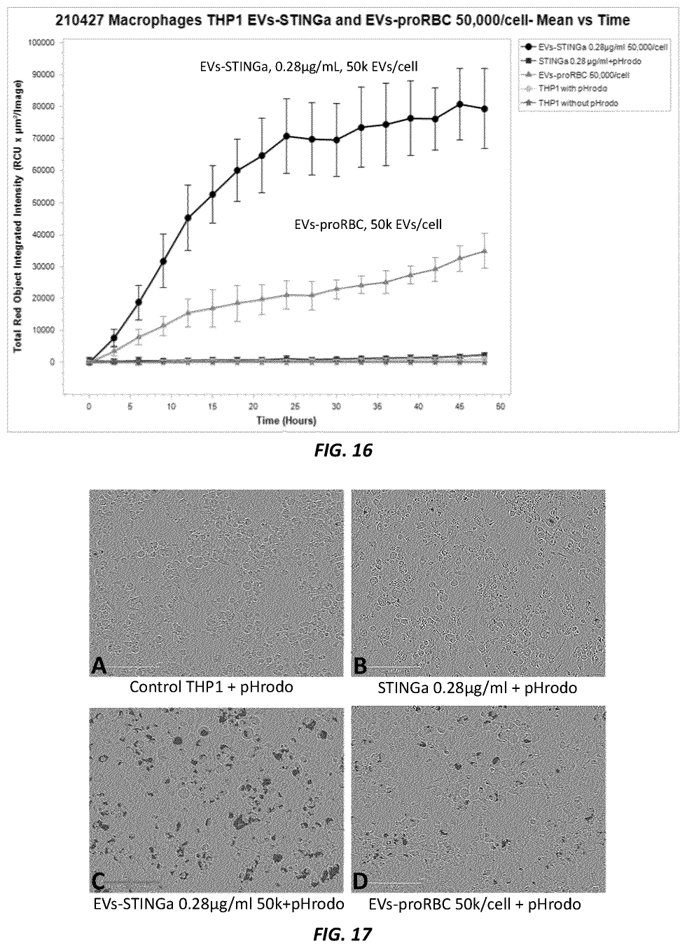

In yet another THP1 macrophage experiment, the following concentrations of active ingredients and EVs were tested: (1) EVs-STINGa 0.28 μg/ml 50k EVs/cell; (2) EVs-STINGa 0.14 μg/ml 25k EVs/cell; (3) EVs-STINGa 0.03 μg/ml 5k EVs/cell; (4) EVs-proRBC 50k, 25k, or 5k EVs/cell; and STINGa 0.28, 0.14, or 0.03 μg/mL+pHrodo were each added to THP1-derived macrophages in triplicate. Control conditions included THP1+pHrodo dye and THP1 without pHrodo dye were incubated for 48 h and the INCUCYTE device was set to read the wells every 30 minutes. As shown in FIG. 16, EVs-STINGa and EVs-proRBC (albeit to a lesser extent) were taken up by THP1 macrophages. And providing a visualization at the cellular level, the FIG. 17 micrographs show the fluorescence observed after 48 h in THP1 macrophages treated with A) control+pHrodo; B) free STINGa 0.28 μg/ml+pHrodo; C) EVs-STINGa 0.28 μg/ml 50k EVs/cell+pHrodo; and D) EVs-proRBC 50k EVs/cell+pHrodo. According to these and other micrographs, EVs-STINGa+pHrodo produced a relatively greater number of red cells than EVs-proRBC+pHrodo. One explanation for this observation is that CLRCEVs according to this disclosure are more readily phagocytosed by macrophages as compared to RCEVs produced from mock-loaded red cells. As such, producing red cell EVs from red cells already containing an active ingredient would appear to confer upon the CLRCEVs an unexpected increase in their capacity for being taken up by phagocytic cells. It is envisioned that such functional differences may be attributable to structural differences imparted on the CLRCEVs by the methods disclosed herein and, to the effect of the active ingredient (STINGa) that may cause heightened activation of THP1-derived macrophages.

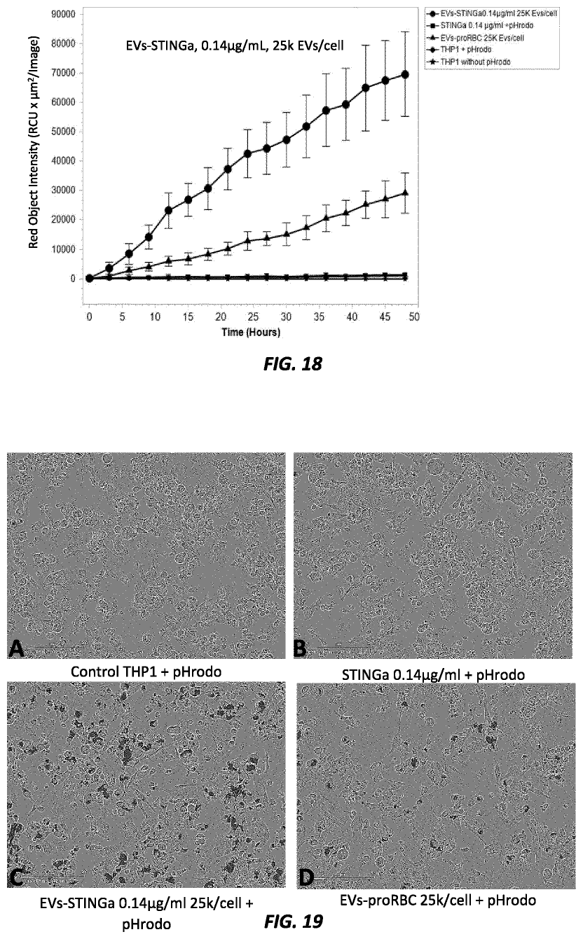

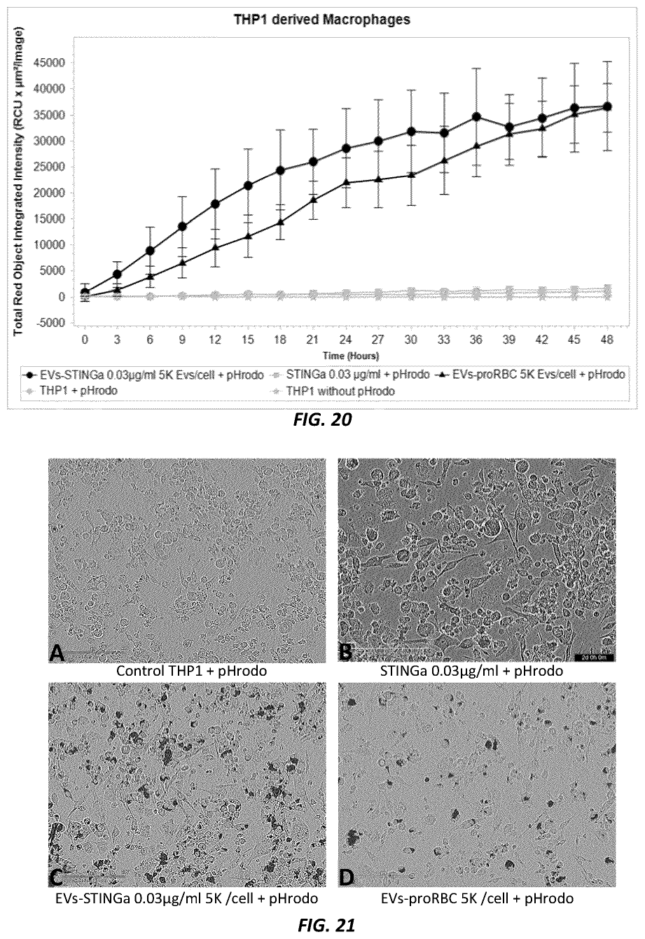

Regarding the other conditions tested, red fluorescence was also detected in THP1-derived macrophages after the addition of EVs-STINGa 0.14 μg/ml 25k EVs/cell; EVs-proRBC 25k EVs/cell; and STINGa 0.14 μg/mL labeled with pHrodo. As indicated in FIG. 18, EVs-STINGa+pHrodo and EVs-proRBC+pHrodo (albeit again, to a much lesser extent) produced red fluorescence, whereas neither free STINGa+pHrodo nor pHrodo alone produced appreciable fluorescence. As before, micrographs show the fluorescence detected 48 h after application of the following: A) pHrodo; B) free STINGa 0.14 μg/ml+pHrodo; C) EVs-STINGa 0.14 μg/ml 25k EVs/cell+pHrodo; and D) EVs-proRBC 25k EVs/cell+pHrodo (FIG. 19). Interestingly, even at this concentration, both EVs-STINGa-pHrodo and EVs-proRBC-pHrodo produced increasing amounts of fluorescence over time. And finally, the graph in FIG. 20 shows the fluorescence detected in THP1-derived macrophages after addition of the lowest concentrations of active according to the following: EVs-STINGa 0.03 μg/ml 5k EVs/cell+pHrodo; and EVs-proRBC 5k EVs/cell+pHrodo; and STINGa 0.03 μg/mL+pHrodo. FIG. 21 are micrographs showing the difference of red fluorescence detected after 48 h in THP1-derived macrophages in A) control condition with pHrodo; B) free STINGa 0.03 μg/ml; C) EVs-STINGa 0.03 μg/ml 5k EVs/cell labeled with pHrodo; and D) EVs-proRBC 5k EVs/cell labeled with pHrodo. Taken together, these results demonstrated a dose response effect based upon the amount of cargo present in EVs-STINGa but not EVs-proRBC, despite the multiple concentrations tested.

This enhanced phagocytosis of EVs-STINGa, coupled with their apparent potential to enhance and/or activate macrophages, suggests that CLRCEVs should be highly effective for in vivo delivery of immune modulators (e.g. STING agonists) or vaccine compositions, including tumor vaccines and vaccines against infectious diseases (e.g. COVID-19). For example, EVs-[immune modulator] could be administered intratumorally and be rapidly and extensively taken up by tumor resident phagocytic cells. In view of the observed enhanced uptake of EVs-STINGa by THP1 macrophages, it is envisioned that EVs-STINGa would have an even stronger efficacy against cancers than the STINGa-loaded red cells described in Example 2.

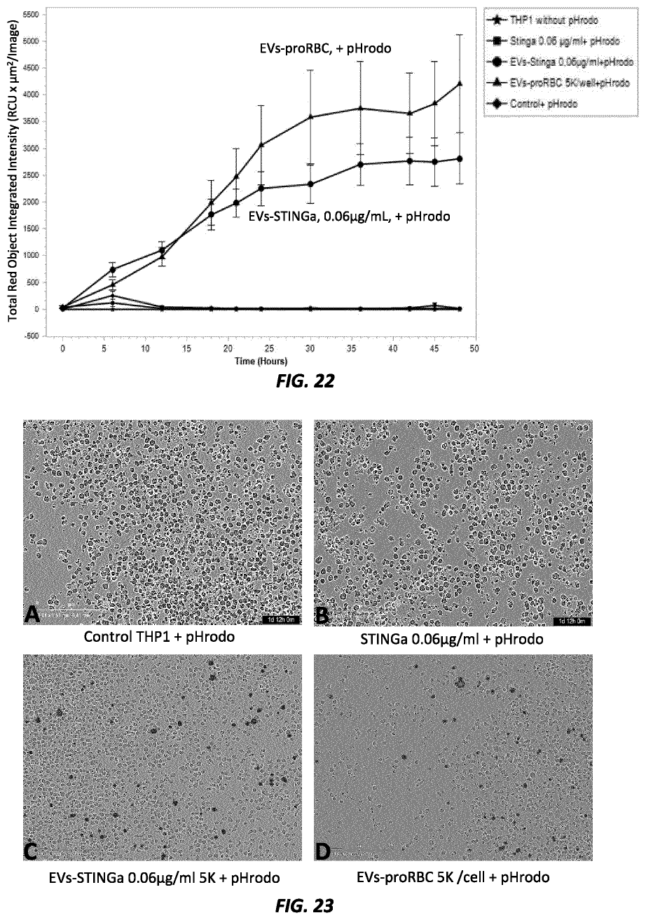

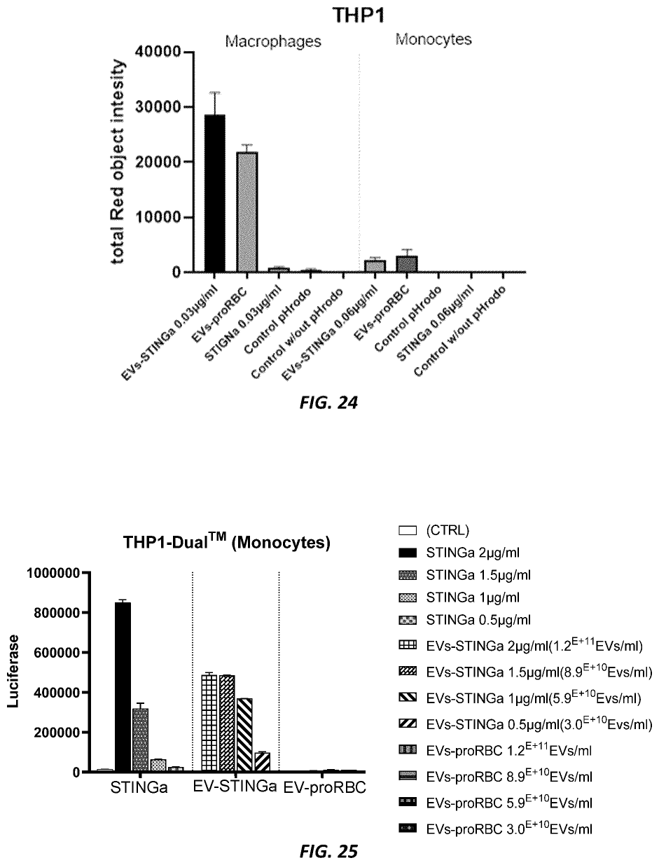

Turning now to the undifferentiated THP1 monocyte studies, FIG. 22 is a graph showing the red fluorescence detected in monocytes after addition of EVs-STINGa 0.06 μg/ml 5k EVs/cell+pHrodo; EVS-proRBC 5k EVs/cell+pHrodo; and STINGa 0.06 μg/mL+pHrodo. FIG. 23 presents micrographs showing the difference of red fluorescence detected after 48 h in THP1 monocytes in A) control condition with pHrodo; B) free STINGa 0.06 μg/ml; C) EVs-STINGa 0.06 μg/ml 5k EVs/cell labeled with pHrodo; and D) EVs-proRBC 5k EVs/cell labeled with pHrodo. FIG. 24 is a graph showing the comparison in the uptake capacity between THP1-derived macrophages and THP1 monocytes seeded at 50k/well and treated with 5k EVs/cell. Histogram bars represent mean fluorescence detected at 48 h after application of the following (left to right): EVs-STINGa 0.03/mL; EVs-proRBC; STINGa 0.03/mL; pHrodo; EVs alone; EVs-STINGa 0.06/mL+pHrodo; EVs-proRBC+pHrodo; pHrodo; STINGa 0.06/mL+pHrodo; EVs alone.

As indicated by the foregoing results, the THP1 monocytes produced far less fluorescence when exposed to the EVs-STINGa and EVs-proRBC as compared with that produced by the differentiated THP1 macrophages. In summary, these studies indicate that 1) the differentiation protocol is effective for the purpose of evaluating the uptake of the disclosed CLRCEVs and RCEVs; and 2) differentiated THP1 cells take up significantly more EV-encapsulated pHrodo as compared with undifferentiated THP1 cells.

### REFERENCES

Daigneault M, et al., (2010) The Identification of Markers of Macrophage Differentiation in PMA-Stimulated THP-1 Cells and Monocyte-Derived Macrophages. PLoS ONE 5(1).
Lund, M. E., et al., The choice of phorbol 12-myristate 13-acetate differentiation protocol influences the response of THP-1 macrophages to a pro-inflammatory stimulus, J. Imm. Methods (2016).

### Example 12—Activation of STING Pathway and Induction of Cytokine Release by STINGa-CLRCEVs

Two different approaches were used to evaluate the effect of the endoplasmic reticulum (ER)-resident protein STimulator of IFN Genes (STING) pathway activation by CLRCEVs.

First, indirect measurements were made using the THP1-Dual™ cells (InvivoGen), which are derived from the human THP-1 monocyte cell line and contain a stably integrated inducible IRF-Lucia luciferase reporter construct that expresses luciferase when STING is activated (e.g. by STING agonists including CDN).

Briefly, 100k cells/well were seeded and then treated with different concentrations of free STING agonist (“STINGa”) (0.5, 1.0, 1.5, and 2.0 μg/mL), EVs-STINGa (also referred to as “ERY-STINGa-CLRCEV” or “STINGa-CLRCEV” throughout) (0.5, 1.0, 1.5, and 2.0 μg/mL, carried by the indicated amounts of EVs), and EVs-proRBC. After 24 h, the supernatants were recovered and subjected to QUANTI-Luc™ detection according to the manufacturer's instructions. As shown in FIG. 25, IRF pathway activation proved to be effective proxy for the amount of STINGa in the various test samples. In this particular experiment, a clear dose response effect was observed for free STINGa (left), whereas the EVs-STINGa produced luciferase activity comparable to 1.5 μg/mL free STINGa at all concentrations except EVs-STINGa 0.5 μg/mL, which still produced detectable activity. No luciferase activity was observed for EVs-proRBC, which indicates that EVs by themselves do not activate the IRF pathway in this reporter system. Notably, comparable doses of STINGa carried by CLRCEV appeared to outperform all but the highest dose of free STINGa. The failure of the highest dose of EVs-STINGa to produce comparable luciferase activity vs. free STINGa data may be explained in part by the poor capacity of THP1 monocytes to take up CLRCEVs (as discussed above). If so, it is envisioned that the in vivo efficacy of EV-STINGa (or any EV-[immune modulator] for that matter) will depend upon the capacity of target cells for taking up the CLRCEVs. It is further envisioned that if the THP1-Dual monocytes had been differentiated into macrophages, the highest dose of EVs-STINGa would have elicited at least as much luciferase activity as the highest dose of free STINGa.

In a second approach, the levels of IL-1b, IL-6, TNF-α, and IFN-β were measured after THP1 macrophages were incubated with varying concentrations of STINGa, STINGa-CLRCEVs, or proRBC EVs.

Briefly, 100k cells/well were seeded and then treated with different concentrations of free STINGa (3 & 8 μg/mL), purified EVs-STINGa (2.0E11 & 7.6E10 EVs/mL), EVs-proRBC (2.0E11 & 7.6E10 EVs/mL), or a single concentration of LPS. Supernatants were collected at 6 h and 24 h after stimulation and frozen until quantification. Supernatants were analyzed by flow cytometry using the BioLegend kit with four (4) different beads recognizing IL-1b, IL-6, TNF-α, IFN-β.

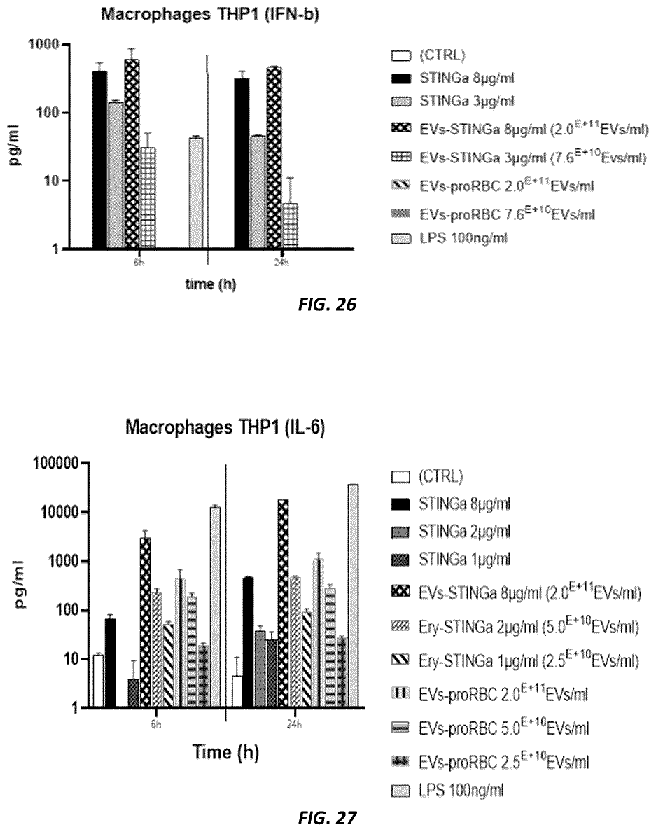

As shown in FIG. 26, free STINGa, EVs-STINGa, and LPS induced IFN-β in THP1 macrophages at 6 hours, but only free STINGa and EVs-STINGa yielded increased IFN-β levels at 24 h. Neither concentration of EVs-proRBC elicited detectable levels of IFN-β at either time point. The induced IFN-β levels are similar to those reported for antigen-presenting cells (APC) and plasmacytoid dendritic cell (pDC) after stimulation with another STING agonist (Deb P et al 2020). In this particular experiment, 8 μg/mL of free STINGa induced 400 pg/ml vs. 593 pg/ml produced by 8 μg/mL EVs-STINGa at 6 h, and after 24 h, the levels of IFN-β remained similar at 318 pg/ml and 463 pg/ml, respectively. At 3 μg/ml, the levels of IFN-β induced by free STINGa were higher compared to EVs-STINGa (140 pg/mL vs. 30 pg/mL).

Release of the Proinflammatory Cytokines IL-6, IL-1b, and TNF-α

It has been reported that vesicles from packed red blood cell (pRBC) units bind to monocytes and induce proinflammatory cytokines, boosting T-cell responses in vitro (Danesh et al 2013). To test whether CLRCEVs and/or RCEVs would likewise induce these cytokines, THP1 macrophages were grown and treated as before and the levels of IL-6, IL-1b, and TNF-α were measured.

As shown in FIG. 27, THP1 macrophages stimulated with free STINGa (1, 2 and 8 μg/ml), EVs-STINGa (1, 2, 8 μg/ml), or EVs-proRBC induced production of IL-6, with either type of EV induced higher levels of IL-6 than did free STINGa. After 24 h, the level of IL-6 induced by EVs was similar to that induced by LPS (37,045 pg/ml) with the higher concentration of EVs-STINGa producing substantially more IL-6 activity (17,613 pg/ml) compared to that produced by EVs-proRBC (1,091 pg/ml) or free STINGa (453 pg/ml). EVs-STINGa induced nearly twice as much IL-6 than did EVs-proRBC at the same concentration of EVs: 484 pg/ml vs 278 pg/ml (at 5.0E10 EVs/mL) and 92 pg/ml vs 27 pg/ml (at 2.5E+10 EVs/mL), respectively. And as regards the two corresponding free STINGa conditions (1 and 2 μg/ml) the levels of IL-6 were statistically similar, at 25 pg/ml and 38 pg/ml, respectively.

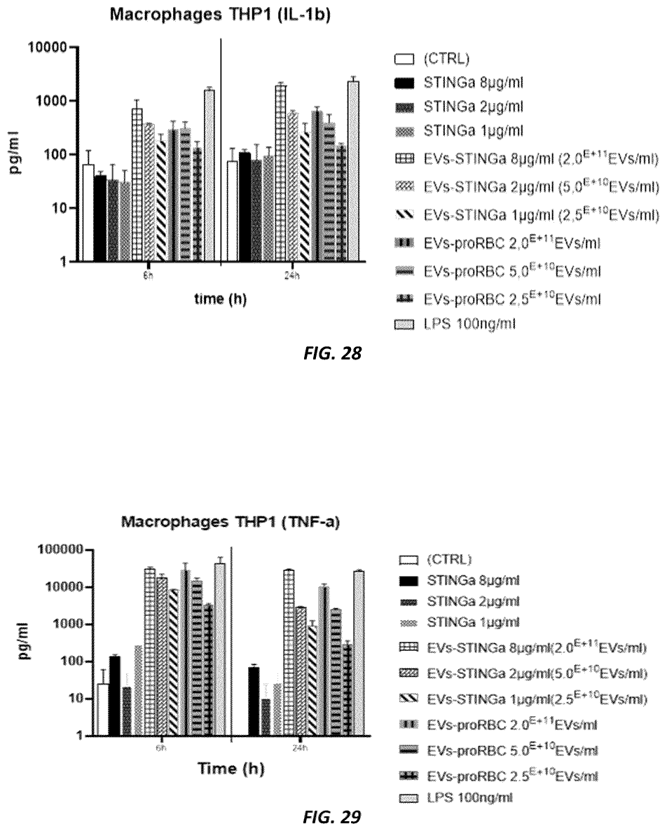

FIG. 28 shows the production of IL-1b under the same experimental conditions as described above. As indicated by the graph, free STINGa did not induce the release of IL-1b and the levels remained similar to control conditions at the 6 h and 24 h time points. In contrast, both EVs-STINGa and EVs-pro RBC induced a dose-dependent response that increased over time and that was slightly higher in THP1 macrophages stimulated with EVs-STINGa. At 24 h, the amounts of IL-1b induced by EVs-STINGa and EVs-proRBC stimulation were 1,951 pg/mL vs. 635 pg/mL (2.0E11 EVs/mL); 603 pg/mL vs. 397 pg/mL (5.0E10 EVs/mL); and 267 pg/mL vs. 155 pg/mL (2.5E10 EVs/mL).

FIG. 29 shows the production of TNFα under the same experimental conditions above. As indicated in the graph, EVs-STINGa and EVs-proRBC induced comparable TNFα levels as compared with that induced by LPS. Additionally, a dose-dependent effect was observed at 24 h and a slightly higher level of TNFα was observed with EVs-STINGa compared to EVs-proRBC across the three concentrations of EVs: 27,519 pg/mL vs. 10,708 pg/mL (2.0E11 EVs/mL); 2,864 pg/mL vs 2470 pg/mL (5.0E10 EVs/mL); and 895 pg/ml vs 295 pg/ml (2.5E10 EVs/mL).

In contrast to the IFN-β results, both EVs-STINGa and LPS were stronger inducers of IL-6 in THP1 macrophages as compared with free STINGa. That said, the results also showed a dose-dependent increase in IL-6 with increasing numbers of proRBC-derived EVs and EVs-STINGa at both the 6 h and 24H time points. Similar inductions were observed for both IL-1b and TNF-α and studies are ongoing to understand the extent to which these inductions are attributable to the EVs themselves or the STINGa contained therein. It is also envisioned that the proRBC-derived EVs could have different characteristics as compared to EVs-STINGa.

Taken together, the results of the phagocytosis and cytokine release studies suggest that CLRCEVs but not empty RCEVs could induce the polarization of macrophages to the M1 phenotype. M1 macrophages participate in inflammatory responses and immune stimulation, and help defend against microbial infection by producing chemokine ligands and proinflammatory cytokines such as TNF-α, IL-1, IL-6, and IL-12, and Type I interferons (IFNs). Four of these cytokines were induced by STINGa-CLRCEVs to a much greater extent than by free STINGa. Studies are ongoing to understand the extent to which these inductions are attributable to the EVs themselves or the STINGa contained therein. Studies are also ongoing to evaluate the extent to which STINGa-CLRCEVs are capable of mobilizing and/or activating macrophages.

### REFERENCES

Deb P, et al. Triggering of the cGAS-STING Pathway in Human Plasmacytoid Dendritic Cells Inhibits TLR9-Mediated IFN Production. J Immunol. 2020 Jul. 1; 205(1):223-236.
Lu, C.-H. et al. (2018). Involvement of M1 Macrophage Polarization in Endosomal Toll-Like Receptors Activated Psoriatic Inflammation. Mediators Inflamm. 2018, 3523642.

### Example 13—Vesiculation by Other Methods

Vesiculation of red blood cells can be performed by a variety of approaches including chemical and physical methods. However, prior to the present disclosure, it was not known whether cargo-loaded red cells (CLRCs) could be subjected to suitable vesiculation conditions to produce useful cargo-loaded red cell extracellular vesicles (CLRCEVs). In the preceding Examples, inventors have presented evidence showing that hypotonically loaded red cells can be vesiculated by applying a mechanical agitation process, which had previously been shown to be capable of increasing the production of exosomes in nucleated mammalian cells. Moreover, these CLRCEVs exhibited useful biological properties including the ability to deliver cargo to macrophages and the ability to activate the macrophages when the cargo comprised an immune modulator. The current studies thus establish a proof-of-concept for the new “Ery-VIP” process: 1) red cell encapsulation (e.g. using ERYCAPS® and/or a modified variation thereof); 2) chemical and/or physical vesiculation; 3) isolation of the CLRCEVs; and 4) preservation of CLRCEVs.

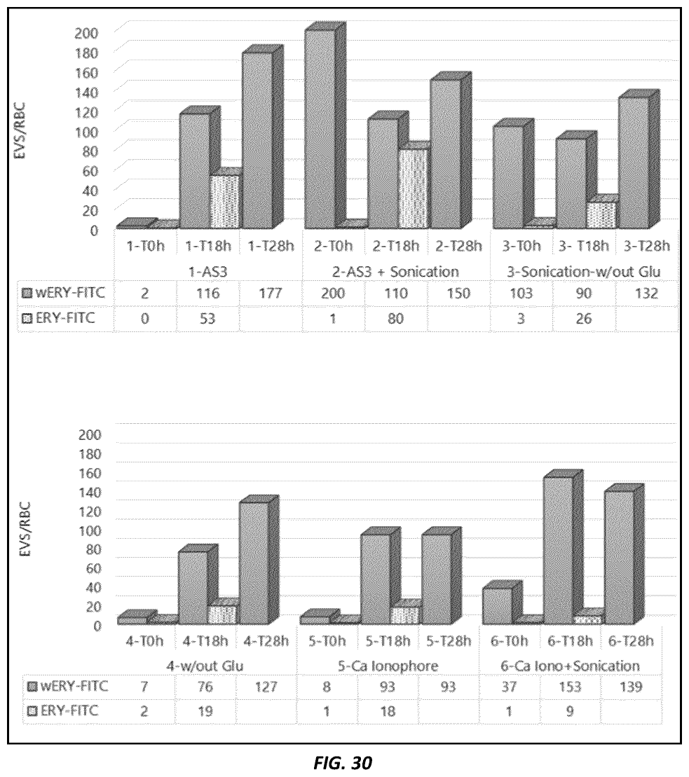

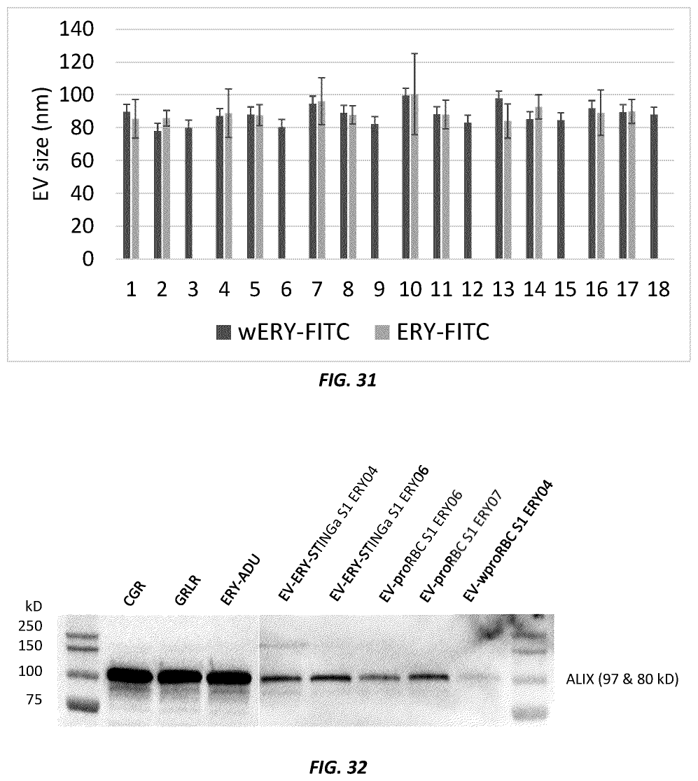

In order to optimize the production of CLRCEVs from CLRCs, several additional vesiculation techniques were tested and the results are summarized in FIGS. 30-31.

FIG. 30 is a graph showing the number of EVs per RBC produced by the following conditions: (1) T0H-AS3; (2) T18H-AS3; (3) T28H-AS3; (4) T0H-AS3-Sonication; (5) T18H-AS3-Sonication; (6) T28H-AS3-Sonication; (7) T0H-PBS-Sonication; (8) T18H-PBS-Sonication; (9) T28H-PBS-Sonication; (10) TO-PBS; (11) T18-PBS; (12) T28-PBS; (13) T0H-calcium ionophore; (14) T18H-calcium ionophore; (15) T28H-calcium ionophore; (16) T0H-calcium ionophore-sonication; (17) T18H-calcium ionophore-sonication; (18) T28H-calcium ionophore-sonication. For wERY-FITC, red cells were subjected to hypotonic encapsulation conditions but not hypertonic resealing conditions during the encapsulation process. Red cells used for the production of wERY-FITC and ERY-FITC came from the same human donor.

FIG. 31 is a graph showing the mean particle size of EVs produced using the following conditions: (1) T0H-AS3; (2) T18H-AS3; (3) T28H-AS3; (4) T0H-AS3-sonication; (5) T18H-AS3-sonication; (6) T28H-AS3-sonication; (7) T0H-PBS-sonication; (8) T18H-PBS-sonication; (9) T28H-PBS-sonication; (10) TO-PBS; (11) T18-PBS; (12) T28-PBS; (13) T0H-calcium ionophore; (14) T18H-calcium ionophore; (15) T28H-calcium ionophore; (16) T0H-calcium ionophore-sonication; (17) T18H-calcium ionophore-sonication; (18) T28H-calcium ionophore-sonication. For wERY-FITC, red cells were subjected to hypotonic encapsulation conditions but not hypertonic resealing conditions during the encapsulation process. Red cells used for the production of wERY-FITC and ERY-FITC came from the same human donor.

For each time point (e.g. 0, 18, 28), red cells containing FITC dextran (DEX-CLRC) were subjected to the conditions indicated above. The following means were tested for their ability to produce useful DEX-CLRCEVs: temperature (37° C.), agitation (24 rpm), sonication (42k Hz), glucose deprivation and calcium ionophore. As indicated above, these methods were used separately or in combination.

In more detail, DEX-CLRCs were suspended to a concentration of about 3.5 billion cells/mL in AS3, PBS (glucose deprivation) or in PBS supplemented with calcium ionophore. Each 110 ml of AS3 solution contained 1.21 g dextrose, 0.647 g sodium citrate, 0.451 g NaCl, 0.304 g monobasic sodium phosphate, 0.046 g citric acid, and 0.033 g adenine. Next, cells were incubated at 37° C. under agitation at 24 rpm for OH, 18H or 28H. At T0, 1 mL was collected and centrifugated at 2000 g for 10 min at 4° C. For the conditions with sonication, sound energy was applied before centrifugation for 42k Hz. At T18H and T28H, 2 mL were collected and centrifugated 2000 g for 10 min at 4° C. The supernatants putatively containing CLRCEVs were analyzed using NTA to evaluate the efficacy of vesiculation using these different conditions (FIG. 30). As was observed in Example 10, the production of EVs from wERY-FITC was substantially (up to 200 times) more effective than from ERY-FITC. In other words, irrespective of the subsequent vesiculation technique, hypotonically loaded red cells produce a significantly larger number of CLRCEVs when they are not subjected to a hypertonic resealing step. Instead, it was surprisingly found that transferring the hypotonically loaded red cells directly to isotonic conditions primed them for vesiculation.

This result may be explained in part by the fact that the hypertonic resealing step had been originally designed and optimized to help restore enzyme-encapsulated red cells to a metabolically healthy state. Red cells processed with this restorative resealing step would be under significantly less stress as compared with cells that had been transferred to isotonic (i.e. about 275 to about 300 mOsm) conditions. It is thus envisioned that omitting the 37° C. isotonic resealing step prevents CLRCs from reestablishing a healthy metabolism after hypotonic loading, thereby artificially aging the CLRCs and priming them for subsequent vesiculation. Now that this disclosure has been made, the skilled person can routinely test various vesiculation means, and combinations thereof, to determine the optimal conditions to produce a desired CLRCEV from its corresponding CLRC. For example, other frequencies and/or durations of sound energy could be usefully applied in the practice of the methods disclosed herein. Furthermore, it is expected that any red cells, irrespective of origin or contents, may be primed for vesiculation by subjecting them to hypotonic conditions and then to isotonic conditions, but not to restorative hypertonic resealing conditions.

### Example 14—Evaluation of CLRCEV Markers

Compared to other reported red blood cell extracellular vesicles, CLRCEVs produced using hypotonic loading (optionally followed by isotonic resealing) may exhibit a specific and distinct profile of markers. To address this question, various ImageStream and marker-specific Western blots were performed.

According to preliminary results, CD235a (RBC marker), CD9, CD83, and CD63 (tetraspanins) may be heterogeneously present in the plurality of CLRCEVs. Moreover, the expression of these markers may be conditioned by their size and the presence or absence cargo. As such, once the vesiculation parameters are further optimized to minimize size differences between EVs and maximize the number of EVs that contain cargo, it is expected that the marker profile will resolve to a consistent signature for CLRCEVs made according to the methods disclosed herein. In some embodiments, CLRCEVs are CD235+ and Cargo+ but neither CD9+, CD83+, nor CD63+.

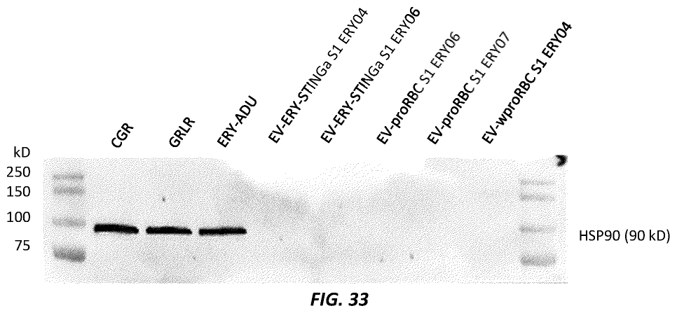

In other experiments, the expression of markers including ALIX and HSP90 were evaluated by conducting Western blots on EVs-STINGa, EVs-proRBC, wproRBC, ERY-STINGa, proRBC and pRBC. The results are presented in FIGS. 32 and 33.

FIG. 32 are Western blots probed with ALIX-specific antibodies. Lanes: (1) ladder; (2) unloaded red cells; (3) mock-loaded proRBC; (4) ERY-STINGa (red cells encapsulated with STINGa using ERYCAPS®; (5) EV-ERY-STINGa, S1 ERY04 batch; (6) EV-ERY-STINGa, S1 ERY06 batch; (7) EV-proRBC S1 ERY06 batch; (8) EV-proRBC S1 ERY07 batch; (9) EV-wproRBC S1 ERY04 batch; (10) ladder;

FIG. 33 are Western blots probed with HSP90-specific antibodies. Lanes: (1) ladder; (2) unloaded red cells; (3) mock-loaded proRBC; (4) ERY-STINGa (red cells encapsulated with STINGa using ERYCAPS®; (5) EV-ERY-STINGa, S1 ERY04 batch; (6) EV-ERY-STINGa, S1 ERY06 batch; (7) EV-proRBC S1 ERY06 batch; (8) EV-proRBC S1 ERY07 batch; (9) EV-wproRBC S1 ERY04 batch; (10) ladder.

As shown in FIG. 32, ALIX was successfully detected in CLRCEV or empty RCEV samples, albeit in different amounts. And as shown in FIG. 33, the same samples were tested to detect HSP90, but they were all negative. TSG101 was also detected in some of samples by western blot (data not shown).

Further, a preliminary test to compare the phagocytosis rate in the presence or absence of anti-CD47 may suggest that CLRCEVs or empty RCEVs do not comprise CD47. Studies are also ongoing to the extent the characterization of CLRCEVs and empty RCEVs, to determine the expression of phosphatidylserine, CD55, CD59, and CD47.

### REFERENCES

Pring F A, et al. (2013) Tetraspanins CD81 and CD82 Facilitate a4131-Mediated Adhesion of Human Erythroblasts to Vascular Cell Adhesion Molecule-1. PLoS ONE 8(5): e62654.
Thangaraju, K, et al. Extracellular Vesicles from Red Blood Cells and Their Evolving Roles in Health, Coagulopathy and Therapy. Int. J. Mol. Sci. 2021, 22, 153.

The application will now be described in the following non-limiting Embodiments.

### EMBODIMENTS OF THE DISCLOSURE

Embodiment 1. A plurality of pharmaceutically acceptable cargo-loaded red cell extracellular vesicles (CLRCEVs) comprising on average at least about 500, 600, 700, 800, 900, 1,000, 2,000, 3,000, 4,000, 5,000, 10,000, 20,000, 30,000, 40,000, 50,000 or about 100,000 molecules of a cargo per CLRCEV; wherein at least about 70%, 80% or 90%, or at least 91%, 92%, 93%, 94% or 95% of the plurality of CLRCEVs are about 100 nm to about 300 nm, or about 100 to about 250 nm, or about 100 to about 200 nm in diameter; and

wherein the CLRCEVs are produced according to the following process:
a) loading red cells with the cargo to produce cargo-loaded red cells (CLRCs) or otherwise providing CLRCs;
b) subjecting the CLRCs to vesiculation to produce CLRCEVs; and
c) isolating the CLRCEVs, thereby producing the plurality of CLRCEVs;
   optionally wherein the CLRCEVs comprise on average 10,000 to 1,000,000 or 100,000 to 700,000 molecules cargo;
   optionally wherein the loading method comprises hypotonic loading and isotonic resealing but not restorative hypertonic resealing;
   optionally wherein when the CLRCs are metabolically healthy, such that they are substantially similar to healthy red cells circulating in an animal including a human, said healthy red cells are treated with hypotonic conditions prior to or during vesiculation step (b).

Embodiment 2. The CLRCEVs of embodiment 1, wherein the cargo is selected from an active pharmaceutical ingredient (API), an API-precursor, a nucleic acid, a peptide, a protein, a small molecule, a gene therapy combination, and combinations thereof.

Embodiment 3. The CLRCEVs of embodiment 2, wherein the cargo is a nucleic acid selected from an mRNA, a gRNA, an antisense oligonucleotide, a microRNA, a siRNA, a circular self-replicating RNA, an expression plasmid, and combinations thereof. In some embodiments, the nucleic acid may comprise a circular RNA, which may be more stable inside cells and/or vesicles than linear RNA.

Embodiment 4. The CLRCEVs of embodiment 2 or 3, wherein the nucleic acid contains non-naturally occurring modifications.

Embodiment 5. The CLRCEVs of any of the preceding embodiments, wherein the cargo comprises at least one component of a gene editing system.

Embodiment 6. The CLRCEVs of any one of the preceding embodiments, for use in a method of treatment.

Embodiment 7. A method of treatment, the method comprising administering an effective amount of the CLRCEVs of embodiment 1 to a subject or patient in need of treatment therewith.

Embodiment 8. Use of the CLRCEVs of any one embodiments 1-6 in the manufacture of a medicament for the treatment of a disease or disorder.

Embodiment 9. The use of embodiment 8, wherein the disease or disorder is selected from a cancer, a genetic disorder, a gastrointestinal disease, a musculoskeletal disease, an immune disorder, an autoimmune disorder, an inflammatory disease, a cardiovascular disease, and a neurological disorder.

Embodiment 10. The use of embodiment 9, wherein the subject or patient is suffering from a cancer selected from ALL, AML, adrenocortical adenoma, anaplastic thyroid cancer, bladder cancer, bone cancer, brain cancer, breast cancer, CLL, chondrosarcoma, colon cancer, colorectal cancer (CRC), DLBCL, Ewing sarcoma, fallopian tube cancer, gallbladder cancer, gastrointestinal/stomach (GIST) cancer, glioblastoma, glioma, hepatoblastoma, hepatocellular carcinoma (HCC), hepatocholangiocarcinoma, kidney cancer, leukemia, liver cancer, lung cancer, lymphoma, medulloblastoma, myeloma, nasopharyngeal cancer, neurofibromatosis-1 associated malignant peripheral nerve sheath tumors (MPNST), osteosarcoma, ovarian cancer, ovarian epithelial cancer, pancreatic cancer, pancreatic ductal carcinoma or pancreatic adenocarcinoma, papillary serous cystadenocarcinoma, prostate cancer, rectal cancer, renal cell carcinoma, rhabdomyosarcoma, salivary gland cancer, soft tissue and bone synovial sarcoma, squamous cell carcinoma of the head and neck (SCCHN), testicular cancer, uterine papillary serous carcinoma (UPSC) or Waldenstrom's macroglobulinemia.

Embodiment 11. A method for producing the plurality of CLRCEVs of embodiment 1 comprising:
(a) providing a plurality of CLRCs, comprising on average at least about 5,000 units of cargo per CLRC;
(b) subjecting the CLRCs to vesiculation; and
(c) isolating the resulting CLRCEVs, thereby producing the plurality of CLRCEVs.

Embodiment 12. The method of embodiment 11, wherein the red cells are loaded using any of the following general encapsulation techniques: hypotonic loading, mechanical loading, microfluidic loading, soluporation, laser-assisted loading, loading via cell-penetrating peptide (CPP), electroporation, transfection, genetic expression, or combinations thereof.

Embodiment 13. The method of embodiment 11, wherein the red cells are loaded and resealed using the following method:
(a) producing an isotonic red cell suspension by placing a sufficient amount of the red cells in a substantially isotonic solution such that the red cell suspension has a hematocrit level greater than about 40%, 50%, 60% or about 65%, and wherein the temperature is maintained from about 1° C. to about 8° C., preferably between about 2° C. and about 6° C.;
(b) contacting the red cell suspension with the cargo;
(c) subjecting the red cell suspension to a lysis solution, wherein the osmolarity of the lysis solution is between about 20 mOsm/L to about 120 mOsm/L or about 70 to about 110 mOsm/L or about 80 to about 100 mOsm/L or about 90 mOsm/L;
(d) allowing the cargo to enter the red cells;
(e) performing steps (b) to (d) such that a combined suspension comprising the red cells and the cargo are separated from the lysis solution via a dialysis membrane; and
(f) resealing the CLRCs by subjecting the combined suspension to a hypertonic solution having and a temperature of from about 25° C. to about 40° C.

Embodiment 14. The method of embodiment 11, wherein the red cells are loaded using microfluidic squeezing.

Embodiment 15. The method of embodiment 11, wherein the red cells are loaded using microfluidic vortex shedding (μVS).

Embodiment 16. The method of embodiment 11, wherein the red cells are loaded using one of the following techniques: electroporation, cell-penetrating peptide (CPP) or lipid nanoparticle.

Embodiment 17. The method of embodiment 11, wherein the red cells are loaded using any or any hypotonic loading approach.

Embodiment 18. The method of embodiment 11, wherein the CLRCs are, or are produced from, engineered red cell precursor cells.

Embodiment 19. The method of embodiment 11, wherein the CLRCs are, or are produced from, engineered reticulocytes.

Embodiment 20. The method of embodiment 11, wherein the CLRCs are vesiculated to form cargo-loaded RCEVs (CLRCEVs) by:
a. providing a plurality of CLRCs suspended in a liquid medium;
b. using an agitator to agitate the liquid medium for a sufficient amount of time to induce the production of a sufficient amount of RCEVs, preferably wherein the time is at least about thirty minutes; and
c. controlling the agitator to cause the liquid medium to flow, the Kolmogorov length of the flow being less than or equal to about 75 pm and preferably about 50 pm, thereby vesiculating the CLRCs to produce CLRCEVs.

As recited in the patent application (WO 2019/002608 A1 herein incorporated by reference in its entirety), the “Kolmogorov length (or Kolmogorov dimension or eddy length) is the length from which the viscosity of a fluid allows dissipating the kinetic energy of a flow of this fluid. In practice, the Kolmogorov length corresponds to the size of the smallest vortices in a turbulent flow. This length Lk is calculated in the publication of Kolmogorov (Kolmogorov, A. N., 1941, January, The local structure of turbulence in incompressible viscous fluid for very large Reynolds numbers, In Dokl. Akad. Nauk, SSSR, Vol. 30, No. 4, pp. 301-305) and described by the following formula (1): Lₖ−v^{3/4}·ε^{-1/4} (1) wherein v is the kinematic viscosity of the flowing liquid medium and £ is the energy dissipated in the fluid per unit of mass (or rate of energy injection into the fluid).

Embodiment 21. The method of embodiment 20, wherein the agitator comprises a blade impeller arranged in a container and moved by a magnetic force transmission system, wherein the speed of the blade impeller is controllable to cause the liquid medium to flow in a laminar or turbulent manner.

Embodiment 22. The method of embodiment 21, wherein the rotation speed of the agitator is capable of being controlled at 100 rpm, the diameter of a blade impeller is about 8 to about 12 cm and the volume of the liquid medium is about 300 to about 500 mL.

Embodiment 23. The method of any one of embodiments 20-22, wherein vesiculation-enhancing components are added to enhance the production of CLRCEVs from cargo-loaded red cells.

Embodiment 24. The method of embodiment 23, wherein the vesiculation enhancing component is selected from calcium and calcium ionophore. In general, CLRCs may be vesiculated by contacting them with any suitable vesicle inducing agent, such as calcium ionophore, lysophosphatidic acid (LPA), or phorbol-12-myristat-13-acetate (PMA). CLRCs may also advantageously be vesiculated by subjecting them to appropriate intensities and durations of mechanical energy, including the methods disclosed in WO 2019/002608.

Embodiment 25. The method of embodiment 20, wherein the liquid medium is sufficiently hypotonic to enhance the production of CLRCEVs, but not so hypotonic as to lyse the red cells completely and release all of the cargo into the liquid medium.

Embodiment 26. The method of embodiment 20, wherein the CLRCEVs comprise on average about 100, 200, 300, 400, 500, 600, 700, 800, 900, 1000 or more units of cargo per RCEV.

Embodiment 27. The method of embodiment 20, wherein the CLRCEVs comprise on average more than 1,000 units of cargo per CLRCEV.

Embodiment 28. The method of embodiment 20, wherein greater than 70% of the CLRCEVs comprise at least 100, 200, 300, 400, 500, 600, 700, 800, 900, 1000 or more units of cargo per CLRCEV.

Embodiment 29. The method of embodiment 28, wherein greater than 80% of the CLRCEVs comprise at least 100, 200, 300, 400, 500, 600, 700, 800, 900, 1000 or more units of cargo per CLRCEV.

Embodiment 30. The method of embodiment 29, wherein greater than 90% of the CLRCEVs comprise at least 100, 200, 300, 400, 500, 600, 700, 800, 900, 1000 or more units of cargo per CLRCEV.

Embodiment 31. A pharmaceutically acceptable composition comprising an effective amount of the CLRCEVs of embodiment 20.

Embodiment 32. A pharmaceutically acceptable composition comprising an effective amount of the CLRCEVs of embodiment 20.

Embodiment 33. The composition of embodiment 32, for use in treating ALL, AML, bladder cancer, bone cancer, breast cancer, CLL, colorectal cancer, DLBCL, gastrointestinal cancer, glioblastoma, glioma, kidney cancer, leukemia, liver cancer, lung cancer, lymphoma, myeloma, nasopharyngeal cancer, pancreatic cancer, renal cell carcinoma, etc.

Embodiment 34. The composition of embodiment 32, for use in stimulating an immune response suppressed by a suppressive tumor environment and/or microenvironment.

Embodiment 35. The composition of embodiment 32, for use in delivering a nucleic acid encoding a therapeutic protein to a target cell in vivo in a subject or patient.

Embodiment 36. The composition of embodiment 32, wherein the CLRCEVs contain an immunostimulant (including CDN or other adjuvant), an immunosuppressant, a CDN, an exogenous nucleic acid or an exogenous polypeptide.

Embodiment 37. The composition of embodiment 36, wherein the CLRCEVs contain and immunostimulant.

Embodiment 38. The composition of embodiment 37, wherein the immunostimulant is a sting agonist.

Embodiment 39. The composition of embodiment 38, wherein the sting agonist is a CDN.

Embodiment 40. The composition of embodiment 39, wherein the CDN is selected from . . . .

Embodiment 41. The CLRCEV of embodiment 20 comprising an exogenous polypeptide.

Embodiment 42. The CLRCEV of embodiment 41, wherein the CLRC was produced by introducing nucleic acid into a red cell precursor cell and allowing the precursor cell to express the exogenous polypeptide.

Embodiment 43. The CLRCEV of embodiment 42, wherein the red cell precursor cell is a megakaryocyte, a hematopoietic stem cell (HSC), an embryonic stem cell (ESC) or an induced pluripotent stem cell (iPSC).

Embodiment 44. The CLRCEV of embodiment 42, wherein the nucleic acid was introduced by subjecting the red cell precursor cell to a transient, controlled cell injury.

Embodiment 45. The CLRCEV of embodiment 44, wherein the cell injury comprised perturbation of the cell membrane.

Embodiment 46. The CLRCEV of embodiment 45, wherein the cell injury comprised sonoporation, electroporation, soluporation, laser-assisted transfection, cell deformation or substance-assisted transfection.

Embodiment 47. The CLRCEV of embodiment 46, wherein the cell injury comprises cell deformation.

Embodiment 48. The CLRCEV of embodiment 47, wherein the cell injury comprises cell squeezing.

Embodiment 49. The CLRCEV of embodiment 42, which was produced by introducing the nucleic acid into a red cell precursor cell using viral transduction.

Embodiment 50. The CLRCEV of embodiment 42, wherein the nucleic acid comprises DNA.

Embodiment 51. The CLRCEV of embodiment 42, wherein the nucleic acid comprises RNA.

Embodiment 52. The CLRCEV of embodiment 41, wherein the polypeptide comprises an antibody, an enzyme, a hormone, a membrane-associated receptor, a nuclear hormone receptor, a cytokine, a heavy-metal scavenger, or a functional fragment thereof.

Embodiment 53. The CLRCEV of embodiment 41, wherein the exogenous polypeptide contained within the intravesicular compartment of the CLRCEV.

Embodiment 54. The CLRCEV of embodiment 53, wherein the polypeptide is less than 1% associated with the inner surface of the CLRCEV.

Embodiment 55. The CLRCEV of embodiment 41, wherein the polypeptide is associated with the surface of the CLRCEV.

Embodiment 56. The CLRCEV of embodiment 41, wherein the polypeptide comprises a transmembrane domain.

Embodiment 57. The CLRCEV of embodiment 41, which comprises at least about 500, 600, 700, 800, 900 or 1,000 copies of the polypeptide.

Embodiment 58. The CLRCEV of embodiment 41, which was produced from a human red cell or a human red cell precursor cell.

Embodiment 59. A pharmaceutical composition comprising the CLRCEVs of embodiment 1 or 41 and optionally a pharmaceutically acceptable excipient and/or carrier.

Embodiment 60. A method of producing the CLRCEV of embodiment 41, comprising: introducing into a nucleated red cell precursor cell an exogenous nucleic acid encoding the exogenous polypeptide; culturing the nucleated red cell precursor cell to produce a CLRC, inducing vesiculation of the CLRC, thereby producing the CLRCEV.

Embodiment 61. The method of embodiment 60, wherein the vesiculation is accomplished using the following steps: a. providing a plurality of CLRCs suspended in a liquid medium;

b. using an agitator to agitate the liquid medium for a sufficient amount of time to induce the production of a sufficient amount of RCEVs, preferably wherein the time is at least about thirty minutes; and

c. controlling the agitator to cause the liquid medium to flow, the Kolmogorov length of the flow being less than or equal to about 75 pm and preferably about 50 μm, thereby vesiculating the CLRCs to produce CLRCEVs.

Embodiment 62. The CLRCEV of embodiment 60, wherein the cargo comprises a viral antigen.

Embodiment 63. The method of embodiment 60, wherein the CLRC is subjected to hypotonic conditions prior to or during the vesiculation.

Embodiment 64. The method of embodiment 63, wherein the hypotonic conditions comprise about 150 to about 250 mOsm, about 160 to about 240 mOsm, or about 170 to about 230 mOsm. Embodiment 65. The method of embodiment 63, wherein the CLRCs are subjected to isotonic conditions after the hypotonic conditions.

Embodiment 66. A device for producing the CLRCEV of any of the preceding embodiments, comprising the following components:

1) a red cell loading means for producing cargo-loaded red cells (CLRCs);

2) a red cell vesiculation means for producing cargo-loaded red cell extracellular vesicles (CLRCEVs) from CLRCs;

3) a CLRCEV isolation means for isolating the CLRCEVs from the residual CLRC materials.

Embodiment 67. The device of embodiment 66, wherein the vesiculation means is capable of subjecting loaded red cells to mechanical energy.

Embodiment 68. The device of embodiment 67, wherein the vesiculation means is capable of delivering an optimal amount of energy to the loaded red cells to maximize any one or more or all of the following: (1) yield of CLRCEVs per CLRC; (2) average amount of cargo per CLRCEV; (3) speed of production of CLRCEVs from CLRCs; (4) uniformity of CLRCEV size as measured by a relatively low polydispersity index (PDI).

Embodiment 69. The device of any one of embodiments 65-68, wherein the loading means is an ERYCAP® device or any device capable of subjecting red cells to hypotonic conditions, the vesiculation means comprises a means for delivering acoustic or vibrational energy to the CLRCs, and the isolation means comprises filter membrane or a means for separating vesicles using acoustic waves.

Embodiment 70. A method for producing cargo-loaded red cell extracellular vesicles (CLRCEVs) from cargo-loaded red cells (CLRCs) comprising the following steps:

1) providing a plurality of red cells;

2) subjecting the red cells to hypotonic conditions;

3) contacting the red cells with active ingredient;

4) allowing the red cells and the active ingredient to remain in the hypotonic conditions for a sufficient period of time to promote a high percentage of loading (e.g. as evidenced by an about equal concentration of active ingredient both inside and outside of the red cells) and a subsequent high yield of CLRCEVs per CLRC (e.g. more than about 100, 110, 120, 130, 140, 150, 160, 170, 180, 190, 200, or more CLRCEVs per CLRC);

5) changing the hypotonic conditions to isotonic conditions (e.g. about 275 to about 300 mOsm) after the sufficient period of time;

6) not subjecting the cells to hypertonic conditions (e.g. greater than about 350 mOsm);

7) subjecting the loaded red cells (CLRCs) to a vesiculation means (e.g. mechanical energy, electromagnetic energy, vibrational energy, sound energy, and/or a chemical vesiculation means); thereby producing the CLRCEVs from the CLRCs.

Embodiment 71. The method of embodiment 70, wherein the vesiculation is accomplished by the application of vortex or vibrational energy to the CLRCs.

Embodiment 72. The method of embodiment 70, wherein the vesiculation is accomplished by the application of acoustic waves to the CLRCs.

Embodiment 73. The method of embodiment 70, wherein the vesiculation is accomplished by extrusion, optionally wherein the extrusion is carried out by preparing a suspension of CLRCs, and conducting a serial extrusion with the CLRCs by sequentially passing the cells through filters with diminishing micro-size pores to produce sub-cell sized microvesicles retaining the same membrane topology as that of the CLRC.

1. A plurality of pharmaceutically acceptable cargo-loaded red cell extracellular vesicles (CLRCEVs) comprising on average at least 500, 600, 700, 800, 900, 1,000, 2,000, 3,000, 4,000, 5,000, 10,000, 20,000, 30,000, 40,000, 50,000 or about 100,000 molecules of a cargo per CLRCEV;
wherein at least about 70%, 80% or 90%, or at least 91%, 92%, 93%, 94% or 95% of the plurality of CLRCEVs are about 100 nm to about 300 nm, or about 100 to about 250 nm in diameter, or about 100 to about 200 nm, or about 100 to about 150 nm; and
wherein the CLRCEVs are produced according to the following process:
a) loading red cells with the cargo to produce cargo-loaded red cells (CLRCs) or otherwise providing CLRCs;
b) subjecting the CLRCs to vesiculation to produce CLRCEVs; and
c) isolating the CLRCEVs, thereby producing the plurality of CLRCEVs.

2. The CLRCEVs of claim 1, wherein the cargo is selected from an active pharmaceutical ingredient (API), an API-precursor, a nucleic acid, a peptide, a protein, a small molecule, a gene therapy combination, and combinations thereof.

3. The CLRCEVs of claim 2, wherein the cargo is a nucleic acid selected from an mRNA, a gRNA, an antisense oligonucleotide, a microRNA, a siRNA, a circular self-replicating RNA, an expression plasmid, and combinations thereof.

4. The CLRCEVs of claim 2, wherein the nucleic acid contains non-naturally occurring modifications.

5.-15. (canceled)

16. The CLRCEVs of claim 3, wherein the nucleic acid contains non-naturally occurring modifications.

17. The CLRCEVs of claim 1, wherein the cargo comprises at least one component of a gene editing system.

18. The CLRCEVs of claim 2, wherein the cargo comprises at least one component of a gene editing system.

19. The CLRCEVs of claim 3, wherein the cargo comprises at least one component of a gene editing system.

20. A method of treatment, the method comprising administering an effective amount of the CLRCEVs of claim 1 to a subject or patient in need of treatment therewith.

21. The method of claim 20, wherein the disease or disorder is selected from a cancer, a genetic disorder, a gastrointestinal disease, a musculoskeletal disease, an immune disorder, an autoimmune disorder, an inflammatory disease, a cardiovascular disease, and a neurological disorder.

22. The method of claim 21, wherein the subject or patient is suffering from a cancer selected from ALL, AML, adrenocortical adenoma, anaplastic thyroid cancer, bladder cancer, bone cancer, brain cancer, breast cancer, CLL, chondrosarcoma, colon cancer, colorectal cancer (CRC), DLBCL, Ewing sarcoma, fallopian tube cancer, gallbladder cancer, gastrointestinal/stomach (GIST) cancer, glioblastoma, glioma, hepatoblastoma, hepatocellular carcinoma (HCC), hepatocholangiocarcinoma, kidney cancer, leukemia, liver cancer, lung cancer, lymphoma, medulloblastoma, myeloma, nasopharyngeal cancer, neurofibromatosis-1 associated malignant peripheral nerve sheath tumors (MPNST), osteosarcoma, ovarian cancer, ovarian epithelial cancer, pancreatic cancer, pancreatic ductal carcinoma or pancreatic adenocarcinoma, papillary serous cystadenocarcinoma, prostate cancer, rectal cancer, renal cell carcinoma, rhabdomyosarcoma, salivary gland cancer, soft tissue and bone synovial sarcoma, squamous cell carcinoma of the head and neck (SCCHN), testicular cancer, uterine papillary serous carcinoma (UPSC), and Waldenstrom's macroglobulinemia.

23. A method for producing the plurality of CLRCEVs of claim 1 comprising:
(a) providing a plurality of CLRCs, comprising on average at least about 5,000 units of cargo per CLRC;
(b) subjecting the CLRCs to vesiculation; and
(c) isolating the resulting CLRCEVs, thereby producing the plurality of CLRCEVs.

24. The method of claim 23, wherein the red cells are loaded using any of the following general encapsulation techniques: hypotonic loading, mechanical loading, microfluidic loading, soluporation, laser-assisted loading, loading via cell-penetrating peptide (CPP), electroporation, transfection, genetic expression, or combinations thereof.

25. The method of claim 23, wherein the red cells are loaded using hypotonic loading, and the red cells are not subjected to restorative hypertonic resealing conditions after the hypotonic conditions, but are instead transferred to isotonic conditions prior to vesiculation.

26. The method of claim 23, wherein the red cells are loaded and resealed using the following method:
(a) producing an isotonic red cell suspension by placing a sufficient amount of the red cells in a substantially isotonic solution such that the red cell suspension has a hematocrit level greater than about 40%, 50%, 60% or about 65%, and wherein the temperature is maintained from about 1° C. to about 8° C.;
(b) contacting the red cell suspension with the cargo;
(c) subjecting the red cell suspension to a lysis solution, wherein the osmolarity of the lysis solution is between about 20 mOsm/L to about 120 mOsm/L or about 70 to about 110 mOsm/L or about 80 to about 100 mOsm/L or about 90 mOsm/L;
(d) allowing the cargo to enter the red cells;
(e) performing steps (b) to (d) such that a combined suspension comprising the red cells and the cargo are separated from the lysis solution via a dialysis membrane; and
(f) resealing the CLRCs by subjecting the combined suspension to a hypertonic solution, or preferably an isotonic solution, having and a temperature of from about 10° C. to about 40° C.

27. The method of claim 26, wherein the temperature at step a) is maintained from 2° C. to 6° C.

28. The method of claim 23, wherein the red cells are loaded using microfluidic squeezing, microfluidic vortex shedding (μVS), electroporation, cell-penetrating peptide (CPP), lipid nanoparticle, any hypotonic loading, any hypotonic loading that does not use a restorative hypertonic resealing step, or the red cells are produced from engineered red cell precursor cells.

29. The method of claim 28, wherein said engineered red cell precursor cells are engineered reticulocytes.

30. The method of claim 23, wherein the CLRCs are vesiculated to form cargo-loaded RCEVs (CLRCEVs) by:
a. providing a plurality of CLRCs suspended in a liquid medium;
b. agitating, vibrating, and/or sonicating the liquid medium for a sufficient amount of time to induce the production of a sufficient amount of RCEVs, thereby vesiculating the CLRCs to produce CLRCEVs.

31. The method of claim 30, wherein at step b., said sufficient amount of time is at least about five to about thirty minutes.
